SEQUENCE LISTING (372 sequences)

SEQ ID NO: 1 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFVKLKSKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 3 - protein, 157 aa, synthetic construct
YFTKLKSKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 4 - protein, 157 aa, synthetic construct
YFTKLKSKLSVIRNLNDQVLFIDQGNRPLFEDMTDSDMRDNAPRTIFIISMYSDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 5 - protein, 157 aa, synthetic construct
YFVKLKSKLSVIRNLNDQVLFIDQGNRPLFEDMTDSDMRDNAPRTIFIISMYSDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 6 - protein, 157 aa, synthetic construct
YFTKLESKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 7 - protein, 157 aa, synthetic construct
YFTKLESKLSVIRNLNDQVLFIDQGNRPLFEDMTDSDMRDNAPRTIFIISMYSDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 8 - protein, 157 aa, synthetic construct
YFVKLESKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 9 - protein, 157 aa, synthetic construct
YFVKLESKLSVIRNLNDQVLFIDQGNRPLFEDMTDSDMRDNAPRTIFIISMYSDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 10 - protein, 157 aa, synthetic construct
YFVKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 11 - protein, 157 aa, synthetic construct
YFTKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 12 - protein, 157 aa, synthetic construct
YFVKLKSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 13 - protein, 157 aa, synthetic construct
YFTKLKSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 14 - protein, 157 aa, synthetic construct
YFVKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNEE

SEQ ID NO: 15 - protein, 157 aa, synthetic construct
YFTKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNEE

SEQ ID NO: 16 - protein, 157 aa, synthetic construct
YFTKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 17 - protein, 157 aa, synthetic construct
YFGKLRSKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 18 - protein, 157 aa, synthetic construct
YFGKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 19 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDKKSDIIFFQRSVPGHDNKMQFESSSYEGYFLASEKERDLFKLILKKEDELGDRSIMFTVQNED

SEQ ID NO: 20 - protein, 157 aa, synthetic construct
YFIKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 21 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDRIKDTKSDIIFFQRSVPGHDNKMQFESSSYEGYFLASEKERDLFKLILKKEDELGDRSIMFTVQNED

SEQ ID NO: 22 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDHKSDIIFFQRSVPGHDNKMQFESSSYEGYFLASEKERDLFKLILKKEDELGDRSIMFTVQNED

SEQ ID NO: 23 - protein, 157 aa, synthetic construct
YFVKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 25 - protein, 157 aa, synthetic construct
YFGKLLSKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 26 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 27 - protein, 157 aa, synthetic construct
YFGKLYSKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 28 - protein, 157 aa, synthetic construct
YFGKLESKLSVIRNLNDQVLFIDQGNRPLFEDMTDSDMRDNAPRTIFIISMYKDSQPRGQAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 29 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNLD

SEQ ID NO: 30 - protein, 157 aa, synthetic construct
YFGKLESKLSVIRNLNDQVLFIDQGNRPLFEDMTDSDMRDNAPRTIFIISMYKDSQPRGNAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 31 - protein, 157 aa, synthetic construct
YFTKLESKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 32 - protein, 157 aa, synthetic construct
YFGKLRSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 33 - protein, 157 aa, synthetic construct
YFGKLESKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 34 - protein, 157 aa, synthetic construct
YFGKLESKLSVIRNLNDQVLFIDQGNRPLFEDMTDSDMRDNAPRTIFIISMYADSQPRGMAVTISVKSEKISTLSSENKIISFKEMNPPDNIKDKKSDIIFFQRSVPGHDNKMQFESSSYEGYFLASEKERDLFKLILKKEDELGDRSIMFTVQNED

SEQ ID NO: 35 - protein, 157 aa, synthetic construct
YFIKLESKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDMRDNAPRTIFIISMYADSQPRGMAVTISVKSEKISTLSSENKIISFKEMNPPDRIKDTKSDIIFFQRSVPGHDNKMQFESSSYEGYFLASEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDMRDNAPRTIFIISMYADSQPRGMAVTISVKSEKISTLSSENKIISFKEMNPPDNIKDHKSDIIFFQRSVPGHDNKMQFESSSYEGYFLASEKERDLFKLILKKEDELGDRSIMFTVQNED

SEQ ID NO: 38 - protein, 157 aa, synthetic construct
YFVKLESKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 40 - protein, 157 aa, synthetic construct
YFGKLLSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDMRDNAPRTIFIISMYADSQPRGMAVTISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 42 - protein, 157 aa, synthetic construct
YFGKLYSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 43 - protein, 157 aa, synthetic construct
YFGKLESKLSVIRNLNDQVLFIDQGNRPLFEDMTDSDMRDNAPRTIFIISMYADSQPRGQAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 44 - protein, 157 aa, synthetic construct
YFGKLESKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNLD

SEQ ID NO: 45 - protein, 157 aa, synthetic construct
YFGKLESKLSVIRNLNDQVLFIDQGNRPLFEDMTDSDMRDNAPRTIFIISMYADSQPRGNAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 46 - protein, 157 aa, synthetic construct
YFTKLKSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 47 - protein, 157 aa, synthetic construct
YFGKLRSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLKSKLSVIRNLNDQVLFIDQGNRPLFEDMTDSDMRDNAPRTIFIISMYKDSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDMRDNAPRTIFIISMYKDSQPRGMAVAISVKSEKISTLSSENKIISFKEMNPPDNIKDKKSDIIFFQRSVPGHDNKMQFESSSYEGYFLASEKERDLFKLILKKEDELGDRSIMFTVQNED

SEQ ID NO: 50 - protein, 157 aa, synthetic construct
YFIKLKSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 51 - protein, 157 aa, synthetic construct
YFGKLKSKLSVIRNLNDQVLFIDQGNRPLFEDMTDSDMRDNAPRTIFIISMYKDSQPRGMAVAISVKSEKISTLSSENKIISFKEMNPPDRIKDTKSDIIFFQRSVPGHDNKMQFESSSYEGYFLASEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDMRDNAPRTIFIISMYKDSQPRGMAVAISVKSEKISTLSSENKIISFKEMNPPDNIKDHKSDIIFFQRSVPGHDNKMQFESSSYEGYFLASEKERDLFKLILKKEDELGDRSIMFTVQNED

SEQ ID NO: 53 - protein, 157 aa, synthetic construct
YFVKLKSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLLSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDMRDNAPRTIFIISMYKDSQPRGMAVAISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLYSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 58 - protein, 157 aa, synthetic construct
YFGKLKSKLSVIRNLNDQVLFIDQGNRPLFEDMTDSDMRDNAPRTIFIISMYKDSQPRGQAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 59 - protein, 157 aa, synthetic construct
YFGKLKSKLSVIRNLNDQVLFIDQGNRPLFEDMTDSDMRDNAPRTIFIISMYKDSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNLD

SEQ ID NO: 60 - protein, 157 aa, synthetic construct
YFGKLKSKLSVIRNLNDQVLFIDQGNRPLFEDMTDSDMRDNAPRTIFIISMYKDSQPRGNAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 61 - protein, 157 aa, synthetic construct
YFTKLKSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 62 - protein, 157 aa, synthetic construct
YFGKLRSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDMRDNAPRTIFIISMYADSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDMRDNAPRTIFIISMYADSQPRGMAVAISVKSEKISTLSSENKIISFKEMNPPDNIKDKKSDIIFFQRSVPGHDNKMQFESSSYEGYFLASEKERDLFKLILKKEDELGDRSIMFTVQNED

SEQ ID NO: 65 - protein, 157 aa, synthetic construct
YFIKLKSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDMRDNAPRTIFIISMYADSQPRGMAVAISVKSEKISTLSSENKIISFKEMNPPDRIKDTKSDIIFFQRSVPGHDNKMQFESSSYEGYFLASEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDMRDNAPRTIFIISMYADSQPRGMAVAISVKSEKISTLSSENKIISFKEMNPPDNIKDHKSDIIFFQRSVPGHDNKMQFESSSYEGYFLASEKERDLFKLILKKEDELGDRSIMFTVQNED

SEQ ID NO: 68 - protein, 157 aa, synthetic construct
YFVKLKSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 69 - protein, 157 aa, synthetic construct
YFGKLKSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 70 - protein, 157 aa, synthetic construct
YFGKLLSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 71 - protein, 157 aa, synthetic construct
YFGKLKSKLSVIRNLNDQVLFIDQGNRPLFEDMTDSDMRDNAPRTIFIISMYADSQPRGMAVAISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 72 - protein, 157 aa, synthetic construct
YFGKLYSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 73 - protein, 157 aa, synthetic construct
YFGKLKSKLSVIRNLNDQVLFIDQGNRPLFEDMTDSDMRDNAPRTIFIISMYADSQPRGQAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 74 - protein, 157 aa, synthetic construct
YFGKLKSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNLD

SEQ ID NO: 75 - protein, 157 aa, synthetic construct
YFGKLKSKLSVIRNLNDQVLFIDQGNRPLFEDMTDSDMRDNAPRTIFIISMYADSQPRGNAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 76 - protein, 157 aa, synthetic construct; identical to SEQ ID NO: 13

SEQ ID NO: 77 - protein, 157 aa, synthetic construct
YFGKLKSKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 78 - protein, 157 aa, synthetic construct
YFGKLKSKLSVIRNLNDQVLFIDQGNRPLFEDMTDSDMRDNAPRTIFIISMYADSQPRGMAVTISVKSEKISTLSSENKIISFKEMNPPDNIKDKKSDIIFFQRSVPGHDNKMQFESSSYEGYFLASEKERDLFKLILKKEDELGDRSIMFTVQNED

SEQ ID NO: 79 - protein, 157 aa, synthetic construct
YFIKLKSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 80 - protein, 157 aa, synthetic construct
YFGKLKSKLSVIRNLNDQVLFIDQGNRPLFEDMTDSDMRDNAPRTIFIISMYADSQPRGMAVTISVKSEKISTLSSENKIISFKEMNPPDRIKDTKSDIIFFQRSVPGHDNKMQFESSSYEGYFLASEKERDLFKLILKKEDELGDRSIMFTVQNED

SEQ ID NO: 81 - protein, 157 aa, synthetic construct
YFGKLKSKLSVIRNLNDQVLFIDQGNRPLFEDMTDSDMRDNAPRTIFIISMYADSQPRGMAVTISVKSEKISTLSSENKIISFKEMNPPDNIKDHKSDIIFFQRSVPGHDNKMQFESSSYEGYFLASEKERDLFKLILKKEDELGDRSIMFTVQNED

SEQ ID NO: 82 - protein, 157 aa, synthetic construct; identical to SEQ ID NO: 12

SEQ ID NO: 83 - protein, 157 aa, synthetic construct
YFGKLKSKLSVIRNLNDQVLFIDQGNRPLFEDMTDSDMRDNAPRTIFIISMYADSQPRGMAVTISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 84 - protein, 157 aa, synthetic construct
YFGKLKSKLSVIRNLNDQVLFIDQGNRPLFEDMTDSDMRDNAPRTIFIISMYADSQPRGQAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 85 - protein, 157 aa, synthetic construct
YFGKLKSKLSVIRNLNDQVLFIDQGNRPLFEDMTDSDMRDNAPRTIFIISMYADSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNLD

SEQ ID NO: 86 - protein, 157 aa, synthetic construct
YFGKLKSKLSVIRNLNDQVLFIDQGNRPLFEDMTDSDMRDNAPRTIFIISMYADSQPRGNAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 87 - protein, 157 aa, synthetic construct
YFGKLESKLSVIRNLNDQVLFIDQGNRPLFEDMTDSDMRDNAPRTIFIISKYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 88 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDFKMQFESSSYEGYFLASEKERDLFKLILKKEDELGDRSIMFTVQNED

SEQ ID NO: 89 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDQKMQFESSSYEGYFLASEKERDLFKLILKKEDELGDRSIMFTVQNED

SEQ ID NO: 90 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKVQFESSSYEGYFLASEKERDLFKLILKKEDELGDRSIMFTVQNED

SEQ ID NO: 91 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKDQFESSSYEGYFLASEKERDLFKLILKKEDELGDRSIMFTVQNED

SEQ ID NO: 92 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKKQFESSSYEGYFLASEKERDLFKLILKKEDELGDRSIMFTVQNED

SEQ ID NO: 93 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKNQFESSSYEGYFLASEKERDLFKLILKKEDELGDRSIMFTVQNED

SEQ ID NO: 94 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKQQFESSSYEGYFLASEKERDLFKLILKKEDELGDRSIMFTVQNED

SEQ ID NO: 95 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKSQFESSSYEGYFLASEKERDLFKLILKKEDELGDRSIMFTVQNED

SEQ ID NO: 96 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKAQFESSSYEGYFLASEKERDLFKLILKKEDELGDRSIMFTVQNED

SEQ ID NO: 97 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKEQFESSSYEGYFLASEKERDLFKLILKKEDELGDRSIMFTVQNED

SEQ ID NO: 98 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRRVPGHDNKMQFESSSYEGYFLASEKERDLFKLILKKEDELGDRSIMFTVQNED

SEQ ID NO: 99 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHNNKMQFESSSYEGYFLASEKERDLFKLILKKEDELGDRSIMFTVQNED

SEQ ID NO: 100 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHTNKMQFESSSYEGYFLASEKERDLFKLILKKEDELGDRSIMFTVQNED

SEQ ID NO: 101 - protein, 157 aa, synthetic construct
YFGKLESKLSVIRNLNDQVLFIDQGNRPLFEDMTDSDMRDNAPRTIFIISRYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 102 - protein, 157 aa, synthetic construct
YFGKLESKLSVIRNLNDQVLFIDQGNRPLFEDMTDSDMRDNAPRTIFIISMYKDSEPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 103 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFARSVPGHDNKMQFESSSYEGYFLASEKERDLFKLILKKEDELGDRSIMFTVQNED

SEQ ID NO: 104 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFVRSVPGHDNKMQFESSSYEGYFLASEKERDLFKLILKKEDELGDRSIMFTVQNED

SEQ ID NO: 105 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHQNKMQFESSSYEGYFLASEKERDLFKLILKKEDELGDRSIMFTVQNED

SEQ ID NO: 106 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHHNKMQFESSSYEGYFLASEKERDLFKLILKKEDELGDRSIMFTVQNED

SEQ ID NO: 107 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHRNKMQFESSSYEGYFLASEKERDLFKLILKKEDELGDRSIMFTVQNED

SEQ ID NO: 108 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDDKMQFESSSYEGYFLASEKERDLFKLILKKEDELGDRSIMFTVQNED

SEQ ID NO: 109 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKIQFESSSYEGYFLASEKERDLFKLILKKEDELGDRSIMFTVQNED

SEQ ID NO: 110 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQLED

SEQ ID NO: 111 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQKED

SEQ ID NO: 112 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQSED

SEQ ID NO: 113 - protein, 157 aa, synthetic construct
YFGKLESKLSVIRNLNDQVLFIDQGNRPLFEDMTDSDMRDNAPRTIFIISMYKDSQPRDMAVTISVKSEKISTLSSENKIISFKEMNPPDNIKDTKSDIIFFQRFVPGHDNKEQFESSSYEGYFLASEKERDLFKLILKKEDELGDRSIMFTVQNED

SEQ ID NO: 114 - protein, 157 aa, synthetic construct
YFGKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQYEE

SEQ ID NO: 115 - protein, 157 aa, synthetic construct
YFGKLESKLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNEE

SEQ ID NO: 116 - protein, 157 aa, synthetic construct
YFGKILSRLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 117 - protein, 157 aa, synthetic construct
YFGKLMSHLSVIRNLNDQVLFIDQGNRPLFEDMTDSDMRDNAPRTIFIISKYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 118 - protein, 157 aa, synthetic construct
YFGKLMSHLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 119 - protein, 157 aa, synthetic construct
YFGKLESHLSVIRNLNDQVLFIDQGNRPLFEDMTDSDMRDNAPRTIFIISMYKDSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNER

SEQ ID NO: 120 - protein, 157 aa, synthetic construct
YFGKILSKLSVIRNLNDQVLFIDQGNRPLFEDMTDSDMRDNAPRTIFIISYYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 121 - protein, 157 aa, synthetic construct
YFGKILSKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 122 - protein, 157 aa, synthetic construct
YFGKILSKLSVIRNLNDQVLFIDQGNRPLFEDMTDSDMRDNAPRTIFIISFYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 123 - protein, 157 aa, synthetic construct
YFGKLSSHLSVIRNLNDQVLFIDQGNRPLFEDMTDSDMRDNAPRTIFIISKYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 124 - protein, 157 aa, synthetic construct
YFGKLSSHLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 125 - protein, 157 aa, synthetic construct
YFGKLQSHLSVIRNLNDQVLFIDQGNRPLFEDMTDSDMRDNAPRTIFIISKYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 126 - protein, 157 aa, synthetic construct
YFGKLQSHLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 127 - protein, 157 aa, synthetic construct
YFGKLTSHLSVIRNLNDQVLFIDQGNRPLFEDMTDSDMRDNAPRTIFIISKYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 128 - protein, 157 aa, synthetic construct
YFGKLTSHLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 129 - protein, 157 aa, synthetic construct
YFGKILSRLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 130 - protein, 157 aa, synthetic construct
YFGKLMSHLSVIRNLNDQVLFIDQGNRPLFEDMTDSDMRDNAPRTIFIISKYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 131 - protein, 157 aa, synthetic construct
YFGKLMSHLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 132 - protein, 157 aa, synthetic construct
YFGKILSKLSVIRNLNDQVLFIDQGNRPLFEDMTDSDMRDNAPRTIFIISYYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 133 - protein, 157 aa, synthetic construct
YFGKILSKLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 134 - protein, 157 aa, synthetic construct
YFGKILSKLSVIRNLNDQVLFIDQGNRPLFEDMTDSDMRDNAPRTIFIISFYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 135 - protein, 157 aa, synthetic construct
YFGKLSSHLSVIRNLNDQVLFIDQGNRPLFEDMTDSDMRDNAPRTIFIISKYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 136 - protein, 157 aa, synthetic construct
YFGKLSSHLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 137 - protein, 157 aa, synthetic construct
YFGKLQSHLSVIRNLNDQVLFIDQGNRPLFEDMTDSDMRDNAPRTIFIISKYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 138 - protein, 157 aa, synthetic construct
YFGKLQSHLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 139 - protein, 157 aa, synthetic construct
YFGKLTSHLSVIRNLNDQVLFIDQGNRPLFEDMTDSDMRDNAPRTIFIISKYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 140 - protein, 157 aa, synthetic construct
YFGKLTSHLSVIRNLNDQVLFIDQGNRPLFEDMTDSDMRDNAPRTIFIISMYADSQPRGMAVAISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 141 - protein, 157 aa, synthetic construct
YFGKLASKLSVIRNLNDQVLFIDQGNRPLFEDMTDSDMRDNAPRTIFIISMYKDSQPRDMAVAISVKSEKISTLSSENKIISFKEMNPPDNIKDTKSDIIFFQRFVPGHDNKEQFESSSYEGYFLASEKERDLFKLILKKEDELGDRSIMFTVQNED

SEQ ID NO: 142 - protein, 157 aa, synthetic construct
YFGKLASKLSVIRNLNDQVLFIDQGNRPLFEDMTDSDMRDNAPRTIFIISMYKDSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQYEE

SEQ ID NO: 143 - protein, 157 aa, synthetic construct
YFGKLASKLSVIRNLNDQVLFIDQGNRPLFEDMTDSDMRDNAPRTIFIISMYKDSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQNEE

SEQ ID NO: 144 - protein, 157 aa, synthetic construct
YFGKLASHLSVIRNLNDQVLFIDQGNRPLFEDMTDSDMRDNAPRTIFIISMYKDSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQNER

SEQ ID NO: 145 - protein, 157 aa, synthetic construct
YFGKLASKLSVIRNLNDQVLFIDQGNRPLFEDMTDSDMRDNAPRTIFIISMYADSQPRDMAVAISVKSEKISTLSSENKIISFKEMNPPDNIKDTKSDIIFFQRFVPGHDNKEQFESSSYEGYFLASEKERDLFKLILKKEDELGDRSIMFTVQNED

SEQ ID NO: 146 - protein, 157 aa, synthetic construct
YFGKLASKLSVIRNLNDQVLFIDQGNRPLFEDMTDSDMRDNAPRTIFIISMYADSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQYEE

SEQ ID NO: 147 - protein, 157 aa, synthetic construct
YFGKLASKLSVIRNLNDQVLFIDQGNRPLFEDMTDSDMRDNAPRTIFIISMYADSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQNEE

SEQ ID NO: 148 - protein, 157 aa, synthetic construct
YFGKLASHLSVIRNLNDQVLFIDQGNRPLFEDMTDSDMRDNAPRTIFIISMYADSQPRGKAVAISVKSEKISTLSSENKIISFKEMNPPDNIKDTKSDIIFFQRSVPGHDNKMQFESSSYEGYFLASEKERDLFKLILKKEDELGDRSIMFTVQNER

SEQ ID NO: 149 - protein, 157 aa, synthetic construct
YFGKLASKLSVIRNLNDQVLFIDQGNRPLFEDMTDSDMRDNAPRTIFIISMYADSQPRDMAVTISVKSEKISTLSSENKIISFKEMNPPDNIKDTKSDIIFFQRFVPGHDNKEQFESSSYEGYFLASEKERDLFKLILKKEDELGDRSIMFTVQNED

SEQ ID NO: 150 - protein, 157 aa, synthetic construct
YFGKLASKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQYEE

SEQ ID NO: 151 - protein, 157 aa, synthetic construct
YFGKLASKLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNEE

SEQ ID NO: 152 - protein, 157 aa, synthetic construct
YFGKLASHLSVIRNLNDQVLFIDQGNRPLFEDMTDSDMRDNAPRTIFIISMYADSQPRGKAVTISVKSEKISTLSSENKIISFKEMNPPDNIKDTKSDIIFFQRSVPGHDNKMQFESSSYEGYFLASEKERDLFKLILKKEDELGDRSIMFTVQNER

SEQ ID NO: 153 - protein, 157 aa, synthetic construct
YFRKLESKLSVIRNLNDQVLFIDQGNRPLFEDMTDSDMRDNAPRTIFIISMYADSQPRGMAVTISVKSEKISTLSSENKIISFKEMNPPDMIKDTKSDIIFFQRSVPGHDNKMQFESSSYEGYFLASEKERDLFKLILKKEDELGDRSIMFTVQNED

SEQ ID NO: 154 - protein, 157 aa, synthetic construct
YFEKLESKLSVIRNLNDQVLFIDQGNRPLFEDMTDSDMRDNAPRTIFIISMYSDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 155 - protein, 157 aa, synthetic construct
YFSKLESKLSVIRNLNDQVLFIDQGNRPLFEDMTDSDMRDNAPRTIFIISMYSDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 156 - protein, 157 aa, synthetic construct
YFAKLESKLSVIRNLNDQVLFIDQGNRPLFEDMTDSDMRDNAPRTIFIISMYSDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 157 - protein, 157 aa, synthetic construct
YFRKLESKLSVIRNLNDQVLFIDQGNRPLFEDMTDSDMRDNAPRTIFIISMYSDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 158 - protein, 157 aa, synthetic construct
YFFKLESKLSVIRNLNDQVLFIDQGNRPLFEDMTDSDMRDNAPRTIFIISMYSDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 159 - protein, 157 aa, synthetic construct
YFTKLESKLSVIRNLNDQVLFIDQGNRPLFEDMTDSDMRDNAPRTIFIISMYSDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 160 - protein, 157 aa, synthetic construct
YFFKLESKLSVIRNLNDQVLFIDQGNRPLFEDMTDSDMRDNAPRTIFIISMYSDSQPRGMAVTISVKSEKISTLSSENKIISFKEMNPPDEIKDTKSDIIFFQRSVPGHDNKMQFESSSYEGYFLASEKERDLFKLILKKEDELGDRSIMFTVQNED

SEQ ID NO: 161 - protein, 157 aa, synthetic construct
YFRKLESKLSVIRNLNDQVLFIDQGNRPLFEDMTDSDMRDNAPRTIFIISMYSDSQPRGMAVTISVKSEKISTLSSENKIISFKEMNPPDAIKDTKSDIIFFQRSVPGHDNKMQFESSSYEGYFLASEKERDLFKLILKKEDELGDRSIMFTVQNED

SEQ ID NO: 162 - protein, 157 aa, synthetic construct
YFRKLESKLSVIRNLNDQVLFIDQGNRPLFEDMTDSDMRDNAPRTIFIISMYADSQPRGMAVTISVKSEKISTLSSENKIISFKEMNPPDSIKDTKSDIIFFQRSVPGHDNKMQFESSSYEGYFLASEKERDLFKLILKKEDELGDRSIMFTVQNED

SEQ ID NO: 163 - protein, 157 aa, synthetic construct
YFHKLESKLSVIRNLNDQVLFIDQGNRPLFEDMTDSDMRDNAPRTIFIISMYSDSQPRGMAVTISVKSEKISTLSSENKIISFKEMNPPDVIKDTKSDIIFFQRSVPGHDNKMQFESSSYEGYFLASEKERDLFKLILKKEDELGDRSIMFTVQNED

SEQ ID NO: 164 - protein, 157 aa, synthetic construct
YFRKLESKLSVIRNLNDQVLFIDQGNRPLFEDMTDSDMRDNAPRTIFIISMYADSQPRGMAVTISVKSEKISTLSSENKIISFKEMNPPDWIKDTKSDIIFFQRSVPGHDNKMQFESSSYEGYFLASEKERDLFKLILKKEDELGDRSIMFTVQNED

SEQ ID NO: 165 - protein, 157 aa, synthetic construct
YFRKLESKLSVIRNLNDQVLFIDQGNRPLFEDMTDSDMRDNAPRTIFIISMYSDSQPRGMAVTISVKSEKISTLSSENKIISFKEMNPPDMIKDTKSDIIFFQRSVPGHDNKMQFESSSYEGYFLASEKERDLFKLILKKEDELGDRSIMFTVQNED

SEQ ID NO: 166 - protein, 157 aa, synthetic construct
YFKKLESKLSVIRNLNDQVLFIDQGNRPLFEDMTDSDMRDNAPRTIFIISMYSDSQPRGMAVTISVKSEKISTLSSENKIISFKEMNPPDIIKDTKSDIIFFQRSVPGHDNKMQFESSSYEGYFLASEKERDLFKLILKKEDELGDRSIMFTVQNED

SEQ ID NO: 167 - protein, 157 aa, synthetic construct
YFAKLESKLSVIRNLNDQVLFIDQGNRPLFEDMTDSDMRDNAPRTIFIISMYSDSQPRGMAVTISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 168 - protein, 157 aa, synthetic construct
YFGKLDSKLSVIRNLNDQVLFIDQGNRPLFEDMTDFDMRDNAPRTIFIISMYTDMQPKGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 169 - protein, 157 aa, synthetic construct
YFGKLESKLSVIRNINDQVLFIDQGNRPLFEDMTDSDMRDNAPRTIFIISMYHDMQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 170 - protein, 157 aa, synthetic construct
YFGKLESKLSVIRNLNDQVLFIDQGNRPLFEDMEDPDMRAGAPRTIFIISMYTDRQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 171 - protein, 157 aa, synthetic construct
YFGKLESKLSVIRNLNDQVLFIDQGNRPLFEDMEDSDMRDDAPRTIFIISMYNDRQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 172 - protein, 157 aa, synthetic construct
YFGKLESKLSVIRNLNDQVLFIDQGNRPLFEDMIDVDMRIMAPRTIFIISMYGDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 173 - protein, 157 aa, synthetic construct
YFGKLESKLSVIRNLNDQVLFIDQGNRPLFEDMNDNDMREGAPRTIFIISMYSDQQPRGMAVTISVKSEKISTLSSENKIISFKEMNPPDNIKDTKSDIIFFQNSVPGYDNKMQFESSSYEGYFLASEKERDLFKLILKKEDELGDRSIMFTVQNED

SEQ ID NO: 174 - protein, 157 aa, synthetic construct
YFGKLESKLSVIRNLNDQVLFIDQGNRPLFEDMNDYDMRHTAPRTIFIISMYGDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 175 - protein, 157 aa, synthetic construct
YFGKLESKLSVIRNLNDQVLFIDQGNRPLFEDMNDYEMRQQAPRTIFIISMYDDDQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 176 - protein, 157 aa, synthetic construct
YFGKLESKLSVIRNLNDQVLFIDQGNRPLFEDMSDLEMRYMAPRTIFIISMYTDKQPRGMAVTISVKSEKISTLSSENKIISFKEMNPPDNIKDTKSDIIFFQRSVKGQDNKMQFESSSYEGYFLASEKERDLFKLILKKEDELGDRSIMFTVQNED

SEQ ID NO: 177 - protein, 157 aa, synthetic construct
YFGKLESKLSVIRNLNDQVLFIDQGNRPLFEDMSDYNMREQAPRTIFIISMYYDIQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 178 - protein, 163 aa, synthetic construct
YFGKLESKLSVIRNLNDQVLFIDQGNRPLFEDMSDYNMREQAPRTIFIISMYYDIQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNEDTVQNED

SEQ ID NO: 179 - protein, 157 aa, synthetic construct
YFGKLESKLSVIRNLNDQVLFIDQGNRPLFEDMTDEEMRVSAPRTIFIISMYYDVQPRGMAVTISVKSEKISTLSSENKIISFKEMNPPDNIKDTKSDIIFFQRSVPGYDNKMQFESSSYEGYFLASEKERDLFKLILKKEDELGDRSIMFTVQNED

SEQ ID NO: 180 - protein, 157 aa, synthetic construct
YFGKLESKLSVIRNLNDQVLFIDQGNRPLFEDMTDFDMRDEAPRTIFIISMYWDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 181 - protein, 157 aa, synthetic construct
YFGKLESKLSVIRNLNDQVLFIDQGNRPLFEDMTDQDMRTNAPRTIFIISMYPDLQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 182 - protein, 157 aa, synthetic construct
  SITE 62: note = X may be deleted
YFGKLESKLSVIRNLNDQVLFIDQGNRPLFEDMTDSDMRDNAPRTIFIISMYXDAQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 183 - protein, 157 aa, synthetic construct
YFGKLESKLSVIRNLNDQVLFIDQGNRPLFEDMTDSDMRDNAPRTIFIISMYDDEQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 184 - protein, 157 aa, synthetic construct
YFGKLESKLSVIRNLNDQVLFIDQGNRPLFEDMTDSDMRDNAPRTIFIISMYDDLQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 185 - protein, 157 aa, synthetic construct
YFGKLESKLSVIRNLNDQVLFIDQGNRPLFEDMTDSDMRDNAPRTIFIISMYDDNQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 186 - protein, 157 aa, synthetic construct
YFGKLESKLSVIRNLNDQVLFIDQGNRPLFEDMTDSDMRDNAPRTIFIISMYDDNQPRGMAVTISVKSEKISTLSSENKIISFKEMNPPDNIKDTKSDIIFFQRSVPGIDNKMQFESSSYEGYFLASEKERDLFKLILKKEDELGDRSIMFTVQNED

SEQ ID NO: 187 - protein, 157 aa, synthetic construct
YFGKLESKLSVIRNLNDQVLFIDQGNRPLFEDMTDSDMRDNAPRTIFIISMYDDTQPRGMAVTISVKSEKISTLSSENKIISFKEMNPPDNIKDTKSDIIFFQRSVPGIDNKMQFESSSYEGYFLASEKERDLFKLILKKEDELGDRSIMFTVQNED

SEQ ID NO: 188 - protein, 157 aa, synthetic construct
YFGKLESKLSVIRNLNDQVLFIDQGNRPLFEDMTDSDMRDNAPRTIFIISMYGDIQPRGMAVTISVKSEKISTLSSENKIISFKEMNPPDNIKDTKSDIIFFQKSVYGHDNKMQFESSSYEGYFLASEKERDLFKLILKKEDELGDRSIMFTVQNED

SEQ ID NO: 189 - protein, 157 aa, synthetic construct
YFGKLESKLSVIRNLNDQVLFIDQGNRPLFEDMTDSDMRDNAPRTIFIISMYGDIQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 190 - protein, 157 aa, synthetic construct
YFGKLESKLSVIRNLNDQVLFIDQGNRPLFEDMTDSDMRDNAPRTIFIISMYGDPQPRGMAVTISVKSEKISTLSSENKIISFKEMNPPDNIKDTKSDIIFFQKSVPGYDNKMQFESSSYEGYFLASEKERDLFKLILKKEDELGDRSIMFTVQNED

SEQ ID NO: 191 - protein, 157 aa, synthetic construct
YFGKLESKLSVIRNLNDQVLFIDQGNRPLFEDMTDSDMRDNAPRTIFIISMYGDVQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 192 - protein, 157 aa, synthetic construct
YFGKLESKLSVIRNLNDQVLFIDQGNRPLFEDMTDSDMRDNAPRTIFIISMYHDGQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 193 - protein, 157 aa, synthetic construct
YFGKLESKLSVIRNLNDQVLFIDQGNRPLFEDMTDSDMRDNAPRTIFIISMYHDHQPRGMAVTISVKSEKISTLSSENKIISFKEMDPPDNIKDTKSDIIFFQRSVPGIDNKMQFESSSYEGYFLASEKERDLFKLILKKEDELGDRSIMFTVQNED

SEQ ID NO: 194 - protein, 157 aa, synthetic construct
YFGKLESKLSVIRNLNDQVLFIDQGNRPLFEDMTDSDMRDNAPRTIFIISMYHDHQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 195 - protein, 157 aa, synthetic construct
YFGKLESKLSVIRNLNDQVLFIDQGNRPLFEDMTDSDMRDNAPRTIFIISMYHDIQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 196 - protein, 157 aa, synthetic construct
YFGKLESKLSVIRNLNDQVLFIDQGNRPLFEDMTDSDMRDNAPRTIFIISMYHDMHPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 197 - protein, 157 aa, synthetic construct
YFGKLESKLSVIRNLNDQVLFIDQGNRPLFEDMTDSDMRDNAPRTIFIISMYHDMQPRGMAVSISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 198 - protein, 157 aa, synthetic construct
YFGKLESKLSVIRNLNDQVLFIDQGNRPLFEDMTDSDMRDNAPRTIFIISMYHDMQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 199 - protein, 157 aa, synthetic construct
YFGKLESKLSVIRNLNDQVLFIDQGNRPLFEDMTDSDMRDNAPRTIFIISMYHDQQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 200 - protein, 157 aa, synthetic construct
YFGKLESKLSVIRNLNDQVLFIDQGNRPLFEDMTDSDMRDNAPRTIFIISMYHDRQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 201 - protein, 157 aa, synthetic construct
YFGKLESKLSVIRNLNDQVLFIDQGNRPLFEDMTDSDMRDNAPRTIFIISMYHDVQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 202 - protein, 157 aa, synthetic construct
YFGKLESKLSVIRNLNDQVLFIDQGNRPLFEDMTDSDMRDNAPRTIFIISMYHDYHPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 203 - protein, 157 aa, synthetic construct
YFGKLESKLSVIRNLNDQVLFIDQGNRPLFEDMTDSDMRDNAPRTIFIISMYHDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 204 - protein, 157 aa, synthetic construct
YFGKLESKLSVIRNLNDQVLFIDQGNRPLFEDMTDSDMRDNAPRTIFIISMYLDEQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 205 - protein, 157 aa, synthetic construct
YFGKLESKLSVIRNLNDQVLFIDQGNRPLFEDMTDSDMRDNAPRTIFIISMYLDGQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 206 - protein, 157 aa, synthetic construct
YFGKLESKLSVIRNLNDQVLFIDQGNRPLFEDMTDSDMRDNAPRTIFIISMYNDHQPRGMAVTISVKSEKISTLSSENKIISFKEMNPPDNIKDTKSDIIFFQRSVPGFDNKMQFESSSYEGYFLASEKERDLFKLILKKEDELGDRSIMFTVQNED

SEQ ID NO: 207 - protein, 157 aa, synthetic construct
YFGKLESKLSVIRNLNDQVLFIDQGNRPLFEDMTDSDMRDNAPRTIFIISMYNDIQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 208 - protein, 157 aa, synthetic construct
YFGKLESKLSVIRNLNDQVLFIDQGNRPLFEDMTDSDMRDNAPRTIFIISMYNDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 209 - protein, 157 aa, synthetic construct
YFGKLESKLSVIRNLNDQVLFIDQGNRPLFEDMTDSDMRDNAPRTIFIISMYQNMHPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 210 - protein, 157 aa, synthetic construct
YFGKLESKLSVIRNLNDQVLFIDQGNRPLFEDMTDSDMRDNAPRTIFIISMYSDFQPRGMAVSISVKSEKISTLSSENKIISFKEMNPPDNIKDTKSDIIFFQKSVPGYDNKMQFESSSYEGYFLASEKERDLFKLILKKEDELGDRSIMFTVQNED

SEQ ID NO: 211 - protein, 157 aa, synthetic construct
YFGKLESKLSVIRNLNDQVLFIDQGNRPLFEDMTDSDMRDNAPRTIFIISMYSDFQPRGMAVTISVKSEKISTLSSENKIISFKEMNPPDNIKDTKSDIIFFQKSVPGYDNKMQFESSSYEGYFLASEKERDLFKLILKKEDELGDRSIMFTVQNED

SEQ ID NO: 212 - protein, 157 aa, synthetic construct
YFGKLESKLSVIRNLNDQVLFIDQGNRPLFEDMTDSDMRDNAPRTIFIISMYSDGQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 213 - protein, 157 aa, synthetic construct
YFGKLESKLSVIRNLNDQVLFIDQGNRPLFEDMTDSDMRDNAPRTIFIISMYSDKQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 214 - protein, 157 aa, synthetic construct
YFGKLESKLSVIRNLNDQVLFIDQGNRPLFEDMTDSDMRDNAPRTIFIISMYSDYQPRGMAVTISVKSEKISTLSSENKIISFKEMNPPDNIKDTKSDIIFFQRSVMGHDNKMQFESSSYEGYFLASEKERDLFKLILKKEDELGDRSIMFTVQNED

SEQ ID NO: 215 - protein, 157 aa, synthetic construct
YFGKLESKLSVIRNLNDQVLFIDQGNRPLFEDMTDSDMRDNAPRTIFIISMYSDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 216 - protein, 157 aa, synthetic construct
YFGKLESKLSVIRNLNDQVLFIDQGNRPLFEDMTDSDMRDNAPRTIFIISMYTDAQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 217 - protein, 157 aa, synthetic construct
YFGKLESKLSVIRNLNDQVLFIDQGNRPLFEDMTDSDMRDNAPRTIFIISMYTDFQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 218 - protein, 157 aa, synthetic construct
YFGKLESKLSVIRNLNDQVLFIDQGNRPLFEDMTDSDMRDNAPRTIFIISMYTDGQPRGMAVTISVKSEKISTLSSENKIISFKEMNPPDNIKDTKSDIIFFQRSVAGQDNKMQFESSSYEGYFLASEKERDLFKLILKKEDELGDRSIMFTVQNED

SEQ ID NO: 219 - protein, 157 aa, synthetic construct
YFGKLESKLSVIRNLNDQVLFIDQGNRPLFEDMTDSDMRDNAPRTIFIISMYTDMQPKGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 220 - protein, 157 aa, synthetic construct
YFGKLESKLSVIRNLNDQVLFIDQGNRPLFEDMTDSDMRDNAPRTIFIISMYTDPQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 221 - protein, 157 aa, synthetic construct
YFGKLESKLSVIRNLNDQVLFIDQGNRPLFEDMTDSDMRDNAPRTIFIISMYWDAQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 222 - protein, 157 aa, synthetic construct
YFGKLESKLSVIRNLNDQVLFIDQGNRPLFEDMTDSDMRDNAPRTIFIISMYYDGHPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 223 - protein, 157 aa, synthetic construct
YFGKLESKLSVIRNLNDQVLFIDQGNRPLFEDMTDSDMRDNAPRTIFIISMYYDGQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 224 - protein, 157 aa, synthetic construct
YFGKLESKLSVIRNLNDQVLFIDQGNRPLFEDMTDSDMRDNAPRTIFIISMYYDVQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 225 - protein, 157 aa, synthetic construct
YFGKLESKLSVIRNLNDQVLFIDQGNRPLFEDMTDSDMRDNAPRTIFIISMYYDYHPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 226 - protein, 157 aa, synthetic construct
YFGKLESKLSVIRNLNDQVLFIDQGNRPLFEDMTDSDMRDNAPRTIFIISMYYDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 227 - protein, 157 aa, synthetic construct
YFGKLESKLSVIRNLNDQVLFIDQGNRPLFEDMTDWEMRFIAPRTIFIISMYDDNQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 228 - protein, 157 aa, synthetic construct
  SITE 40: note = X may be deleted
YFGKLESKLSVIRNLNDQVLFIDQGNRPLFEDMTDYEMRXMAPRTIFIISMYSDLQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 229 - protein, 157 aa, synthetic construct
YFGKLESKLSVIRNLNDQVLFIGQGNRPLFEDMTDSDMRDNAPRTIFIISMYYDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 230 - protein, 157 aa, synthetic construct
  SITE 38: note = X may be deleted
YFGKLESKLSVISNLNDQVLFIHQGNTPLLEDMDDYEXRRMAPRTIFIISMYSDLQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 231 - protein, 157 aa, synthetic construct
YFGKLESKVSVIRNLNDQVLFIDQGNRPLFEDMTDSDMRDNAPRTIFIISMYHDMQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 232 - protein, 157 aa, synthetic construct
YFGKLEYKLSVIRNLNDQVLFIDQGNRPLFEDMTDSDMRDNAPRTIFIISMYHDMQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 233 - protein, 157 aa, synthetic construct
YFGKWESKLSVIRNLNDQVLFMDQGNRPLFEDMTDSDMRDNAPRTIFIISMYNDYHPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 234 - protein, 157 aa, synthetic construct
YFGKWEYKLSVIRNLNDQVLFIDQGNRPLFEDMTDSDMRDNAPRTIFIISMYHDMQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 235 - protein, 157 aa, synthetic construct
YFGKLESKLSVIRNLNDQVLFIDQGNRPLFEDMTDSDMRDNAPRTIFIISMYNDAQPRGMAVTISVKSEKISTLSSENKIISFKEMNPPDNIKDTKSDIIFFQNSVAGYDNKMQFESSSYEGYFLASEKERDLFKLILKKEDELGDRSIMFTVQNED

SEQ ID NO: 236 - protein, 157 aa, synthetic construct
YFGKLESKLSVIRNLNDQVLFIDQGNRPLFEDMIDTDMRAMAPRTIFIISMYTDYQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 237 - protein, 157 aa, synthetic construct
YFGKLESKLSVIRNLNDQVLFIDQGNRPLFEDMLDVDMRASAPRTIFIISMYGDWQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 238 - protein, 157 aa, synthetic construct
YFGKLESKLSVIRNLNDQVLFIDQGNRPLFEDMMDTEMRTMAPRTIFIISMYTDWQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 239 - protein, 157 aa, synthetic construct
YFGKLESKLSVIRNLNDQVLFIDQGNRPLFEDMTDEEMRKTAPRTIFIISMYTDQQPRGMAVTISVKSEKISTLSSENKIISFKEMNPPDNIKDTKSDIIFFQHSVPGHDNKMQFESSSYEGYFLASEKERDLFKLILKKEDELGDRSIMFTVQNED

SEQ ID NO: 240 - protein, 157 aa, synthetic construct
YFGKLESKLSVIRNLNDQVLFIDQGNRPLFEDMTDEEMRRGAPRTIFIISMYYDFQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 241 - protein, 157 aa, synthetic construct
YFGKLESKLSVIRNLNDQVLFIDQGNRPLFEDMTDSDMRDNAPRTIFIISMYDDQQPRGMAVTISVKSEKISTLSSENKIISFKEMNPPDNIKDTKSDIIFFQRSVMGHDNKMQFESSSYEGYFLASEKERDLFKLILKKEDELGDRSIMFTVQNED

SEQ ID NO: 242 - protein, 157 aa, synthetic construct
YFGKLESKLSVIRNLNDQVLFIDQGNRPLFEDMTDSDMRDNAPRTIFIISMYGDVQPRGMAVTISVKSEKISTLSSENKIISFKEMNPPDNIKDTKSDIIFFQYSVPGNDNKMQFESSSYEGYFLASEKERDLFKLILKKEDELGDRSIMFTVQNED

SEQ ID NO: 243 - protein, 157 aa, synthetic construct
YFGKLESKLSVIRNLNDQVLFIDQGNRPLFEDMTDSDMRDNAPRTIFIISMYNDMQPRGMAVTISVKSEKISTLSSENKIISFKEMNPPDNIKDTKSDIIFFQSSVAGHDNKMQFESSSYEGYFLASEKERDLFKLILKKEDELGDRSIMFTVQNED

SEQ ID NO: 244 - protein, 157 aa, synthetic construct
YFGKLESRGCVIRNLNDQVLFIDQGNRPLFEDMTDSDMRDNAPRTIFIISMYGDAQPRGMAVTISVKSEKISTLSSENKIISFKEMNPPDNIKDTKSDIIFFQGSVPGHDNKMQFESSSYEGYFLASEKERDLFKLILKKEDELGDRSIMFTVQNED

SEQ ID NO: 245 - protein, 157 aa, synthetic construct
YFGKLESKLSVIRNLNDQVLFIDQGNRPLFEDMTDSDMRDNAPRTIFIISMYVDQQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 246 - protein, 157 aa, synthetic construct
YFGKLESKLSVIRNLNDQVLFIDQGNRPLFEDMTDSDMRDNAPRTIFIISMYDDQQPRGMAVTISVKSEKISTLSSENKIISFKEMNPPDNIKDTKSDIIFFQRSVPGYDNKMQFESSSYEGYFLASEKERDLFKLILKKEDELGDRSIMFTVQNED

SEQ ID NO: 247 - protein, 157 aa, synthetic construct
YFDKLGSKLSVIRNLNDQVLFIDQGNRPLFEDMTDSDMRDNAPRTIFIISMYGASQPRGMAVSISVKSEKISTLSSENKIISFKEMNPPDYIKDTKSDIIFFQQSVPGHDNKMQFESSSYEGYFLASEKERDLFKLILKKEDELGDRSIMFTVQNED

SEQ ID NO: 248 - protein, 157 aa, synthetic construct
YFDKLQSKLSVIRNLNDQVLFIDQGNSPLFEDMTDSDMRDNAPRTIFIISMYRARQPRGMAVSISVKSEKISTLSSENKIISFKEMNPPDQIKDTKSDIIFFQRSVPGHDNKMQFESSSYEGYFLASEKERDLFKLILKKEDELGDRSIMFTVQNED

SEQ ID NO: 249 - protein, 157 aa, synthetic construct
YFEKLASKLSVIRNLNDQVLFIDQGNRPLFEDMTDSDMRDNAPRTIFIISMYQWVQPRGMAVMISVKSEKISTLSSENKIISFKEMNPPDMIKDTKSDIIFFQNSVPGHDNKMQFESSSYEGYFLASEKERDLFKLILKKEDELGDRSIMFTVQNED

SEQ ID NO: 250 - protein, 157 aa, synthetic construct
YFEKLGSKLSVIRNLNDQVLFIDQGNRPLFEDMTDSDMRDNAPRTIFIISMYKDSQPRGMAVTISVKSEKISTLSSENKIISFKEMNPPDMIKDTKSDIIFFQASVPGHDNKMQFESSSYEGYFLASEKERDLFKLILKKEDELGDRSIMFTVQNED

SEQ ID NO: 251 - protein, 157 aa, synthetic construct
YFEKLHSKLSVIRNLNDQVLFIDQGNSPLFEDMTDSDMRDNAPRTIFIISMYKATQPRGMAVSISVKSEKISTLSSENKIISFKEMNPPDFIKDTKSDIIFFQSSVPGHDNKMQFESSSYEGYFLASEKERDLFKLILKKEDELGDRSIMFTVQNED

SEQ ID NO: 252 - protein, 157 aa, synthetic construct
YFGKLDSKLSVIRNLNDQVLFIDQGNRPLFEDMTDSDMRDNAPRTIFIISMYKTWQPRGMAVSISVKSEKISTLSSENKIISFKEMNPPDSIKDTKSDIIFFQTSVPGHDNKMQFESSSYEGYFLASEKERDLFKLILKKEDELGDRSIMFTVQNED

SEQ ID NO: 253 - protein, 157 aa, synthetic construct
YFGKLESKLSVIRNLNDQVLFIDQGNRPLFEDMTDSDMRDNAPRTIFIISMYKDSQPRGMAVTISVKSEKISTLSSENKIISFKEMNPPDSIKDTKSDIIFFQHSVPGHDNKMQFESSSYEGYFLASEKERDLFKLILKKEDELGDRSIMFTVQNED

SEQ ID NO: 254 - protein, 157 aa, synthetic construct
YFGKLESKLSVIRNLNDQVLFIDQGNRPLFEDMTDSDMRDNAPRTIFIISMYTHVQPRGMAVAISVKSEKISTLSSENKIISFKEMNPPDKIKDTKSDIIFFQRSVPGHDNKMQFESSSYEGYFLASEKERDLFKLILKKEDELGDRSIMFTVQNED

SEQ ID NO: 255 - protein, 157 aa, synthetic construct
YFGKLGSKLSVIRNLNDQVLFIDQGNRPLFEDMTDSDMRDNAPRTIFIISMYKAKQPRGMAVSISVKSEKISTLSSENKIISFKEMNPPDKIKDTKSDIIFFQRSVPGHDNKMRFESSSYEGYFLASEKERDLFKLILKKEDELGDRSIMFTVQNED

SEQ ID NO: 256 - protein, 157 aa, synthetic construct
YFGKLNSKLSVIRNLNDQVLFIDQGNRPLFEDMTDSDMRDNAPRTIFIISMYKNNQPRGMAVTISVKSEKISTLSSENKIISFKEMNPPDHIKDTKSDIIFFQPSVPGHDNKMQFESSSYEGYFLASEKERDLFKLILKKEDELGDRSIMFTVQNED

SEQ ID NO: 257 - protein, 157 aa, synthetic construct
YFGKLTSKLSVIRNLNDQVLFIDQGNRPLFEDMTDSDMRDNAPRTIFIISMYKPKQPRGMAVTISVKSEKISTLSSENKIISFKEMNPPDNIKDTKSDIIFFQSSVPGHDNKMQFESSSYEGYFLASEKERDLFKLILKKEDELGDRSIMFTVQNED

SEQ ID NO: 258 - protein, 157 aa, synthetic construct
YFHKLQSKLSVIRNLNDQVLFIDQGNRPLFEDMTDSDMRDNAPRTIFIISMYNSPQPRGMAVAISVKSEKISTLSSENKIISFKEMNPPDIIKDTKSDIIFFQSSVPGHDNKMQFESSSYEGYFLASEKERDLFKLILKKEDELGDRSIMFTVQNED

SEQ ID NO: 259 - protein, 157 aa, synthetic construct
YFIKLDSKLSVIRNLNDQVLFIDQGNRPLFEDMTDSDMRDNAPRTIFIISMYKAFQPRGMAVTISVKSEKISTLSSENKIISFKEMNPPDYIKDTKSDIIFFQHSVPGHDNKMQFESSSYEGYFLASEKERDLFKLILKKEDELGDRSIMFTVQNED

SEQ ID NO: 260 - protein, 157 aa, synthetic construct
YFIKLQSKLSVIRNLNDQVLFIDQGNRPLFEDMTDSDMRDNAPRTIFIISMYKDSQPRGMAVTISVKSEKISTLSSENKIISFKEMNPPDMIKDTKSDIIFFQYSVPGHDNKMQFESSSYEGYFLASEKERDLFKLILKKEDELGDRSIMFTVQNED

SEQ ID NO: 261 - protein, 157 aa, synthetic construct
YFLKLNSKLSVIRNLNDQVLFMDQGNRPLFEDMTDSDMRDNAPRTIFIISMYKDSQPRGMAVVISVKSEKISTLSSENKIISFKEMNPPDTIKDTKSDIIFFQPSVPGHDNKMQFESSSYEGYFLASEKERDLFKLILKKEDELGDRSIMFTVQNED

SEQ ID NO: 262 - protein, 157 aa, synthetic construct
YFLKLQSKLSVIRNLNDQVLFIDQGNRPLFEDMTDSDMRDNAPRTIFIISMYKDSQPRGMAVHISVKSEKISTLSSENKIISFKEMNPPDMIKDTKSDIIFFQHSVPGHDNKMQFESSSYEGYFLASEKERDLFKLILKKEDELGDRSIMFTVQNED

SEQ ID NO: 263 - protein, 157 aa, synthetic construct
YFLKLQSKLSVIRNLNDQVLFIDQGNRPLFEDMTDSDMRDNAPRTIFIISMYKDSQPRGMAVSISVKSEKISTLSSENKIISFKEMNPPDLIKDTKSDIIFFQPSVPGHDNKMQFESSSYEGYFLASEKERDLFKLILKKEDELGDRSIMFTVQNED

SEQ ID NO: 264 - protein, 157 aa, synthetic construct
YFLKLQSKLSVIRNLNDQVLFIDQGNRPLFEDMTDSDMRDNAPRTIFIISMYKDSQPRGMAVSISVKSEKISTLSSENKIISFKEMNPPDLIKDTKSDIIFFQTSVPGHDNKMQFESSSYEGYFLASEKERDLFKLILKKEDELGDRSIMFTVQNED

SEQ ID NO: 265 - protein, 157 aa, synthetic construct
YFLKLQSKLSVIRNLNDQVLFIDQGNRPLFEDMTDSDMRDNAPRTIFIISMYKYPQPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 266 - protein, 157 aa, synthetic construct
YFLKLSSKLSVIRNLNDQVLFIDQGNRPLFEDMTDSDMRDNAPRTIFIISMYKTSQPRGMAVSISVKSEKISTLSSENKIISFKEMNPPDYIKDTKSDIIFFQQSVPGHDNKMQFESSSYEGYFLASEKERDLFKLILKKEDELGDRSIMFTVQNED

SEQ ID NO: 267 - protein, 157 aa, synthetic construct
YFLKLYSKLSVIRNLNDQVLFIDQGNRPLFEDMTDSDMRDNAPRTIFIISMYKTSQPRGMAVSISVKSEKISTLSSENKIISFKEMNPPDYIKDTKSDIIFFQQSVPGHDNKMQFESSSYEGYFLASEKERDLFKLILKKEDELGDRSIMFTVQNED

SEQ ID NO: 268 - protein, 157 aa, synthetic construct
YFNKLSSKLSVIRNLNDQVLFIDQGNRPLFEDMTDSDMRDNAPRTIFIISMYQWRQPRGMAVTISVKSEKISTLSSENKIISFKEMNPPDTIKDTKSDIIFFQTSVPGHDNKMQFESSSYEGYFLASEKERDLFKLILKKEDELGDRSIMFTVQNED

SEQ ID NO: 269 - protein, 157 aa, synthetic construct
YFNKLYSKLSVIRNLNDQVLFIDQGNRPLFEDMTDSDMRDNAPRTIFIISMYQWRQPRGMAVTISVKSEKISTLSSENKIISFKEMNPPDTIKDTKSDIIFFQPSVPGHDNKMQFESSSYEGYFLASEKERDLFKLILKKEDELGDRSIMFTVQNED

SEQ ID NO: 270 - protein, 157 aa, synthetic construct
YFPKLDSKLSVIRNLNDQVLFIDQGNRPLFEDMTDSDMRDNAPRTIFIISMYTNSQPRGMAVAISVKSEKISTLSSENKIISFKEMNPPDTIKDTKSDIIFFQPSVPGHDNKMQFESSSYEGYFLASEKERDLFKLILKKEDELGDRSIMFTVQNED

SEQ ID NO: 271 - protein, 157 aa, synthetic construct
YFPKLGSKLSVIRNLNDQVLFIDQGNRPLFEDMTDSDMRDNAPRTIFIISMYKPWQPRGMAVTISVKSEKISTLSSENKIISFKEMNPPDEIKDTKSDIIFFQKSVPGHDNKMQFESSSYEGYFLASEKERDLFKLILKKEDELGDRSIMFTVQNED

SEQ ID NO: 272 - protein, 157 aa, synthetic construct
YFPKLGSKLSVIRNLNDQVLFIDQGNRPLFEDMTDSDMRDNAPRTIFIISMYKTWQPRGMAVTISVKSEKISTLSSENKIISFKEMNPPDEIKDTKSDIIFFQKSVPGHDNKMQFESSSYEGYFLASEKERDLFKLILKKEDELGDRSIMFTVQNED

SEQ ID NO: 273 - protein, 157 aa, synthetic construct
YFPKLHSKLSVIRNLNDQVLFIDQGNRPLFEDMTDSDMRDNAPRTIFIISMYKAPKPRGMAVSISVKSEKISTLSSENKIISFKEMNPPDMIKDTKSDIIFFQRSVPGHDNKMQFESSSYEGYFLASEKERDLFKLILKKEDELGDRSIMFTVQNED

SEQ ID NO: 274 - protein, 157 aa, synthetic construct
YFPKLHSKLSVIRNLNDQVLFIDQGNRPLFEDMTDSDMRDNAPRTIFIISMYKDSQPRGMAVTISVKSEKISTLSSENKIISFKEMNPPDLIKDTKSDIIFFQRSVPGHDNKMQFESSSYEGYFLASEKERDLFKLILKKEDELGDRSIMFTVQNED

SEQ ID NO: 275 - protein, 157 aa, synthetic construct
YFPKLISKLSVIRNLNDQVLFIDQGNRPLFEDMTDSDMRDNAPRTIFIISMYNWPQPRGMAVSISVKSEKISTLSSENKIISFKEMNPPDLIKDTKSDIIFFQQSVPGHDNKMQFESSSYEGYFLASEKERDLFKLILKKEDELGDRSIMFTVQNED

SEQ ID NO: 276 - protein, 157 aa, synthetic construct
YFPKLKSKLSVIRNLNDQVLFIDQGNRPLFEDMTDSDMRDNAPRTIFIISMYKRPQPRGMAVAISVKSEKISTLSSENKIISFKEMNPPDVIKDTKSDIIFFQRSVPGHDNKMQFESSSYEGYFLASEKERDLFKLILKKEDELGDRSIMFTVQNED

SEQ ID NO: 277 - protein, 157 aa, synthetic construct
YFPKLKSKLSVIRNLNDQVLFIDQGNRPLFEDMTDSDMRDNAPRTIFIISMYKYPHPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 278 - protein, 157 aa, synthetic construct
YFPKLKSKLSVIRNLNDQVLFIDQGNRPLFEDMTDSDMRDNAPRTIFIISMYKYPQPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 279 - protein, 157 aa, synthetic construct
YFPKLKSKLSVIRNLNDQVLFIDQGNRPLFEDMTDSDMRDNAPRTIFIISMYKYPQPRGMAVSISVKSEKISTLSSENKIISFKEMNPPDSIKDTKSDIIFFQQSVPGHDNKMQFESSSYEGYFLASEKERDLFKLILKKEDELGDRSIMFTVRNED

SEQ ID NO: 280 - protein, 157 aa, synthetic construct
YFPKLKSKLSVIRNLNDQVLFIDQGNRPLFEDMTDSDMRDNAPRTIFIISMYMWNQPRGMAVSISVKSEKISTLSSENKIISFKEMNPPDIIKDTKSDIIFFQRSVPGHDNKMQFESSSYEGYFLASEKERDLFKLILKKEDELGDRSIMFTVQNED

SEQ ID NO: 281 - protein, 157 aa, synthetic construct
YFPKLKSKLSVIRNLNDQVLFIDQGNRPLFEDMTDSDMRDNAPRTIFIISMYNSPQPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 282 - protein, 157 aa, synthetic construct
YFPKLKSKLSVIRNLNDQVLFIDQGNRPLFEDMTDSDMRDNAPRTIFIISMYTYVQPRGMAVIISVKSEKISTLSSENKIISFKEMNPPDIIKDTKSDIIFFQRSVPGHDNKMQFESSSYEGYFLASEKERDLFKLILKKEDELGDRSIMFTVQNED

SEQ ID NO: 283 - protein, 157 aa, synthetic construct
YFPKLNSKLSVIRNLNDQVLFIDQGNRPLFEDMTDSDMRDNAPRTIFIISMYKYSQPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 284 - protein, 157 aa, synthetic construct
YFPKLRSKLSVIRNLNDQVLFIDQGNRPLFEDMTDSDMRDNAPRTIFIISMYKWQQPRGMAVSISVKSEKISTLSSENKIISFKEMNPPDTIKDTKSDIIFFQHSVPGHDNKMQFESSSYEGYFLASEKERDLFKLILKKEDELGDRSIMFTVQNED

SEQ ID NO: 285 - protein, 157 aa, synthetic construct
YFPKLRSKLSVIRNLNDQVLFIDQGNRPLFEDMTDSDMRDNAPRTIFIISMYTYIQPRGMAVVISVKSEKISTLSSENKIISFKEMNPPDGIKDTKSDIIFFQKSVPGHDNKMQFESSSYEGYFLASEKERDLFKLILKKEDELGDRSIMFTVQNED

SEQ ID NO: 286 - protein, 157 aa, synthetic construct
YFPKLSSKLSVIRNLNDQVLFIDQGNRPLFEDMTDSDMRDNAPRTIFIISMYNYPQPRGMAVIISVKSEKISTLSSENKIISFKEMNPPDKIKDTKSDIIFFQHSVPGHDNKMQFESSSYEGYFLASEKERDLFKLILKKEDELGDRSIMFTVQNED

SEQ ID NO: 287 - protein, 157 aa, synthetic construct
YFPKLSSKLSVIRNLNDQVLFIDQGNRPLFEDMTDSDMRDNAPRTIFIISMYRYPQPRGMAVIISVKSEKISTLSSENKIISFKEMNPPDKIKDTKSDIIFFQRSVPGHDNKMQFESSSYEGYFLASEKERDLFKLILKKEDELGDRSIMFTVQNED

SEQ ID NO: 288 - protein, 157 aa, synthetic construct
YFPKLVSKLSVIRNLNDQVLFIDQGNRPLFEDMTDSDMRDNAPRTIFIISMYAWPQPRGMAVSISVKSEKISTLSSENKIISFKEMNPPDLIKDTKSDIIFFQKSVPGHDNKMQFESSSYEGYFLASEKERDLFKLILKKEDELGDRSIMFTVQNED

SEQ ID NO: 289 - protein, 157 aa, synthetic construct
YFPKLVSKLSVIRNLNDQVLFIDQGNRPLFEDMTDSDMRDNAPRTIFIISMYAWPQPRGMAVSISVKSEKISTLSSENKIISFKEMNPPDLIKDTKSDIIFFQKSVPGHDNKMQFESSSYEGYFLASEKERDLFKLILKKEDELGDRSIMFTVRNED

SEQ ID NO: 290 - protein, 157 aa, synthetic construct
YFQKLDSKLSVIRNLNDQVLFIDQGNRPLFEDMTDSDMRDNAPRTIFIISMYSIEQPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 291 - protein, 157 aa, synthetic construct
YFQKLGSKLSVIRNLNDQVLFIDQGNRPLFEDMTDSDMRDNAPRTIFIISMYKNHQPRGMAVVISVKSEKISTLSSENKIISFKEMNPPDMIKDTKSDIIFFQTSVPGHDNKMQFESSSYEGYFLASEKERDLFKLILKKEDELGDRSIMFTVQNED

SEQ ID NO: 292 - protein, 157 aa, synthetic construct
YFQKLHSKLSVIRNLNDQVLFIDQGNRPLFEDMTDSDMRDNAPRTIFIISMYKNIQPRGMAVSISVKSEKISTLSSENKIISFKEMNPPDVIKDTKSDIIFFQRSVPGHDNKMQFESSSYEGYFLASEKERDLFKLILKKEDELGDRSIMFTVQNED

SEQ ID NO: 293 - protein, 157 aa, synthetic construct
YFQKLHSKLSVIRNLNDQVLFIDQGNRPLFEDMTDSDMRDNAPRTIFIISMYTSYQPRGMAVTISVKSEKISTLSSENKIISFKEMNPPDYIKDTKSDIIFFQKSVPGHDNKMQFESSSYEGYFLASEKERDLFKLILKKEDELGDRSIMFTVQNED

SEQ ID NO: 294 - protein, 157 aa, synthetic construct
YFQKLHYKLSVIRNLNDQVLFIAQGNSPLFEDMTDSDMRDNAPRTIFIISMYKSRQPRGMAVFISVKSEKISTLSSENKIISFKEMNPPDFIKDTKSDIIFFQQSVPGHDNKMQFESSSYEGYFLASEKERDLFKLILKKEDELGDRSIMFTVQNED

SEQ ID NO: 295 - protein, 157 aa, synthetic construct
YFQKLKSKLSVIRNLNDQVLFIDQGNRPLFEDMTDSDMRDNAPRTIFIISMYKSPQPRGMAVSISVKSEKISTLSSENKIISFKEMNPPDSIKDTKSDIIFFQQSVPGHDNKMQFESSSYEGYFLASEKERDLFKLILKKEDELGDRSIMFTVQNED

SEQ ID NO: 296 - protein, 157 aa, synthetic construct
YFQKLNSKLSVIRNLNDQVLFIDQGNRPLFEDMTDSDMRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 297 - protein, 157 aa, synthetic construct
YFQKLNSKLSVIRNLNDQVLFIDQGNRPLFEDMTDSDMRDNAPRTIFIISMYKYPQPRGMAVSISVKSEKISTLSSENKIISFKEMNPPDSIKDTKSDIIFFQHSVPGHDNKMQFESSSYEGYFLASEKERDLFKLILKKEDELGDRSIMFTVQNED

SEQ ID NO: 298 - protein, 157 aa, synthetic construct
YFQKLNSKLSVIRNLNDQVLFIDQGNRPLFEDMTDSDMRDNAPRTIFIISMYTASQPRGMAVTISVKSEKISTLSSENKIISFKEMNPPDKIKDTKSDIIFFQPSVPGHDNKMQFESSSYEGYFLASEKERDLFKLILKKEDELGDRSIMFTVQNED

SEQ ID NO: 299 - protein, 157 aa, synthetic construct
YFQKLQSKLSVIRNLNDQVLFIDQGNRPLFEDMTDSDMRDNAPRTIFIISMYTWFQPRGMAVTISVKSEKISTLSSENKIISFKEMNPPDYIKDTKSDIIFFQRSVPGHDNKMQFESSSYEGYFLASEKERDLFKLILKKEDELGDRSIMFTVQNED

SEQ ID NO: 300 - protein, 157 aa, synthetic construct
YFQKLSSKLSVIRNLNDQVLFIDQGNRPLFEDMTDSDMRDNAPRTIFIISMYKDVQPRGMAVAISVKSEKISTLSSENKIISFKEMNPPDTIKDTKSDIIFFQRSVPGHDNKMQFESSSYEGYFLASEKERDLFKLILKKEDELGDRSIMFTVQNED

SEQ ID NO: 301 - protein, 157 aa, synthetic construct
YFRKLDSKLSVIRNLNDQVLFIDQGNRPLFEDMTDSDMRDNAPRTIFIISMYKAQQPRGMAVSISVKSEKISTLSSENKIISFKEMNPPDKIKDTKSDIIFFQPSVPGHDNKMQFESSSYEGYFLASEKERDLFKLILKKEDELGDRSIMFTVQNED

SEQ ID NO: 302 - protein, 157 aa, synthetic construct
YFRKWHSKLSVIRNLNDQVLFIDQGNRPLFEDMTDSDMRDNAPRTIFIISMYKYSHPRGMAVTISVKSEKISTLSSENKIISFKEMNPPDRIKDTKSDIIFFQRSVPGHDNKMQFESSSYEGYFLASEKERDLFKLILKKEDELGDRSIMFTVQNED

SEQ ID NO: 303 - protein, 157 aa, synthetic construct
YFSKLGSKLSVIRNLNDQVLFIDQGNRPLFEDMTDSDMRDNAPRTIFIISMYKVKQPRGMAVSISVKSEKISTLSSENKIISFKEMNPPDYIKDTKSDIIFFQTSVPGHDNKMQFESSSYEGYFLASEKERDLFKLILKKEDELGDRSIMFTVQNED

SEQ ID NO: 304 - protein, 157 aa, synthetic construct
YFSKLHSKLSVIRNLNDQVLFIDQGNRPLFEDMTDSDMRDNAPRTIFIISMYYPYQPRGMAVSISVKSEKISTLSSENKIISFKEMNPPDAIKDTKSDIIFFQSSVPGHDNKMQFESSSYEGYFLASEKERDLFKLILKKEDELGDRSIMFTVQNED

SEQ ID NO: 305 - protein, 157 aa, synthetic construct
YFSKLQSKLSVIRNLNDQVLFIDQGNRPLFEDMTDSDMRDNAPRTIFIISMYNTNQPRGMAVAISVKSEKISTLSSENKTISFKEMNPPDSIKDTKSDIIFFQRSVPGHDNKMQFESSSYEGYFLASEKERDLFKLILKKEDELGDRSIMFTVQNED

SEQ ID NO: 306 - protein, 157 aa, synthetic construct
YFSKLQSKLSVIRNLNDQVLFIDQGNRPLFEDMTDSDMRDNAPRTIFIISMYSYHQPRGMAVTISVKSEKISTLSSENKIISFKEMNPPDLIKDTKSDIIFFQQSVPGHDNKMQFESSSYEGYFLASEKERDLFKLILKKEDELGDRSIMFTVQNED

SEQ ID NO: 307 - protein, 157 aa, synthetic construct
YFTKLQSKLSVIRNLNDQVLFIDQGNRPLFEDMTDSDMRDNAPRTIFIISMYGGHQPRGMAVAITVKSEKISTLSSENKIISFKEMNPPDRIKDTKSDIIFFQQSVPGHDNKMQFESSSYEGYFLASEKERDLFKLILKKEDELGDRSIMFTVQNED

SEQ ID NO: 308 - protein, 157 aa, synthetic construct
YFVKLQSKLSVIRNLNDQVLFIDQGNRPLFEDMPDSDMRDNAPRTIFIISMYKMFQPRGMAVTISVKSEKISTLSSENKIISFKEMNPPDRIKDTKSDIIFFQHSVPGHDNKMQFESSSYEGYFLASEKERDLFKLILKKEDELGDRSIMFTVQNED

SEQ ID NO: 309 - protein, 157 aa, synthetic construct
YFYKLNSKLSVIRNLNDQVLFIDQGNRPLFEDMTDSDMRDNAPRTIFIISMYKDSQPRGMAVTISVKSEKISTLSSENKIISFKEMNPPDYIKDTKSDIIFFQSSVPGHDNKMQFESSSYEGYFLASEKERDLFKLILKKEDELGDRSIMFTVQNED

SEQ ID NO: 310 - protein, 157 aa, synthetic construct
YFYKLSSKLSVIRNLNDQVLFIDQGNRPLFEDMTDSDMRDNAPRTIFIISMYSWLQPRGMAVSISVKSEKISTLSSENKIISFKEMNPPDIIKDTKSDIIFFQRSVPGHDNKMQFESSSYEGYFLASEKERDLFKLILKKEDELGDRSIMFTVQNED

SEQ ID NO: 311 - protein, 157 aa, synthetic construct
YFYKLSSKLSVIRNLNDQVLFIDQGNRPLFEDMTDSDMRDNAPRTIFIISMYSWLQPRGMAVTISVKSEKISTLSSENKIISFKEMNPPDIIKDTKSDIIFFQRSVPGHDNKMQFESSSYEGYFLASEKERDLFKLILKKEDELGDRSIMFTVQNED

SEQ ID NO: 312 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQGLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVGSKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDCLSVFLNQLCVLHEKTPVSDRVTKCCTESLVNGRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 313 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 233 aa, Mus musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 315 - protein, 232 aa, Homo sapiens
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 232 aa, Homo sapiens
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 317 - protein, 232 aa, Homo sapiens
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 318 - protein, 232 aa, Homo sapiens
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 319 - protein, 210 aa, Homo sapiens
NGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVNLTCLVKGFYPSDIAVEWESNGQPENNYKTTPPVLDSDGSFFLNSTLTVDKSRWQQGNVFSCSVMHEALHNHYTQKSLSLS

SEQ ID NO: 320 - protein, 28 aa, synthetic construct
GGSGGGGSGGGSGGGGSGGGGSGGGSGG

SEQ ID NO: 321 - protein, 15 aa, synthetic construct
GGGGSGGGGSGGGGS

SEQ ID NO: 322 - protein, 20 aa, synthetic construct
GGGSGGGGSGGGSGGGGSGG

SEQ ID NO: 323 - protein, 5 aa, synthetic construct
GGSGG

SEQ ID NO: 324 - protein, 20 aa, synthetic construct
MYRMQLLSCIALSLALVTNS

SEQ ID NO: 325 - protein, 36 aa, synthetic construct
MAAEPVEDNCINFVAMKFIDNTLYFIAEDDENLESD

SEQ ID NO: 326 - protein, 35 aa, synthetic construct
MAAMSEDSCVNFKEMMFIDNTLYFIPEENGDLESD

SEQ ID NO: 327 - protein, 20 aa, synthetic construct
GGGGSGGGGSGGGGSGGGGS

SEQ ID NO: 328 - protein, 10 aa, synthetic construct
GGGSGGGGSG

SEQ ID NO: 329 - protein, 10 aa, synthetic construct
GGSGGGGSGG

SEQ ID NO: 330 - protein, 5 aa, synthetic construct
DGGGS

SEQ ID NO: 331 - protein, 5 aa, synthetic construct
TGEKP

SEQ ID NO: 332 - protein, 4 aa, synthetic construct
GGRR

SEQ ID NO: 333 - protein, 5 aa, synthetic construct
  REGION >5: note = The entire sequence of amino acids 1-5 can be repeated one to five times
GGGGS

SEQ ID NO: 334 - protein, 14 aa, synthetic construct
EGKSSGSGSESKVD

SEQ ID NO: 335 - protein, 18 aa, synthetic construct
KESGSVSSEQLAQFRSLD

SEQ ID NO: 336 - protein, 8 aa, synthetic construct
GGRRGGGS

SEQ ID NO: 337 - protein, 9 aa, synthetic construct
LRQRDGERP

SEQ ID NO: 338 - protein, 12 aa, synthetic construct
LRQKDGGGSERP

SEQ ID NO: 339 - protein, 18 aa, synthetic construct
GSTSGSGKPGSGEGSTKG

SEQ ID NO: 340 - protein, 4 aa, synthetic construct
  REGION >4: note = The entire sequence of amino acids 1-4 can be repeated one to ten times
GGGS

SEQ ID NO: 341 - protein, 5 aa, synthetic construct
  REGION >5: note = The entire sequence of amino acids 1-5 can be repeated one to ten times
GGGGS

SEQ ID NO: 342 - protein, 7 aa, synthetic construct
  REGION >7: note = The entire sequence of amino acids 1-7 can be repeated one to ten times
SGGSGGG

SEQ ID NO: 343 - protein, 7 aa, synthetic construct
  REGION >7: note = The entire sequence of amino acids 1-7 can be repeated one to ten times
GGSGGSG

SEQ ID NO: 344 - protein, 5 aa, synthetic construct
  REGION >4: note = The entire sequence of amino acids 1-5 can be repeated two or four times
GGGGS

SEQ ID NO: 345 - protein, 15 aa, synthetic construct
GLNDIFEAQKIEWHE

SEQ ID NO: 346 - protein, 6 aa, synthetic construct
HHHHHH

SEQ ID NO: 347 - protein, 6 aa, synthetic construct
GGSGGG

SEQ ID NO: 348 - protein, 27 aa, synthetic construct
GGSHHHHHHGGSGLNDIFEAQKIEWHE

SEQ ID NO: 349 - protein, 29 aa, synthetic construct
GGSGGHHHHHHGGSGLNDIFEAQKIEWHE

SEQ ID NO: 350 - protein, 27 aa, synthetic construct
GGSGLNDIFEAQKIEWHEGGSHHHHHH

SEQ ID NO: 351 - protein, 157 aa, synthetic construct
NFTRLKSTTAVIRNINDQVLFVDKRQPVFEDMTDIDQSASEPQTRLIIYMYADSEVRGLAVTLSVKDSKMSTLSSKNKIISFEEMDPPENIDDIQSDLIFFQKRVPGHNKMEFESSLYEGHFLACQKEDDAFKLILKKKDENGDKSVMFTLTNLHQS

SEQ ID NO: 352 - protein, 157 aa, synthetic construct
NFVRLKSTTAVIRNINDQVLFVDKRQPVFEDMTDIDQSASEPQTRLIIYMYADSEVRGLAVTLSVKDSKMSTLSSKNKIISFEEMDPPENIDDIQSDLIFFQKRVPGHNKMEFESSLYEGHFLACQKEDDAFKLILKKKDENGDKSVMFTLTNLHQS

SEQ ID NO: 353 - protein, 157 aa, synthetic construct
NFTRLHSTTAVIRNINDQVLFVDKRQPVFEDMTDIDQSASEPQTRLIIYMYADSEVRGLAVTLSVKDSKMSTLSSKNKIISFEEMDPPENIDDIQSDLIFFQKRVPGHNKMEFESSLYEGHFLACQKEDDAFKLILKKKDENGDKSVMFTLTNLHQS

SEQ ID NO: 354 - protein, 157 aa, synthetic construct
NFVRLHSTTAVIRNINDQVLFVDKRQPVFEDMTDIDQSASEPQTRLIIYMYADSEVRGLAVTLSVKDSKMSTLSSKNKIISFEEMDPPENIDDIQSDLIFFQKRVPGHNKMEFESSLYEGHFLACQKEDDAFKLILKKKDENGDKSVMFTLTNLHQS

SEQ ID NO: 355 - protein, 157 aa, synthetic construct
NFGRLHSTTAVIRNINDQVLFVDKRQPVFEDMTDIDQSASEPQTRLIIYMYKDSEVRGLAVTLSVKDSKMSTLSTKNKIISFEEMDPPENIDDIQSDLIFFQKRVPGHNKMEFESSLYEGHFLATQKEDDAFKLILKKKDENGDKSVMFTLTNLHQS

SEQ ID NO: 356 - protein, 157 aa, synthetic construct
NFGRLHWTTAVIRNINDQVLFVDKRQPVFEDMTDIDQSASEPQTRLIIYMYKDSEVRGLAVTLSVKDSKMSTLSGKNKIISFEEMDPPENIDDIQSDLIFFQKRVPGHNKMEFESSLYEGHFLADQKEDDAFKLILKKKDENGDKSVMFTLTNLHQS

SEQ ID NO: 357 - protein, 157 aa, synthetic construct
NFGRLHWTTAVIRNINDQVLFVDKRQPVFEDMTDIDQSASEPQTRLIIYMYKDSEVRGLAVTLSVKDSKMSTLSTKNKIISFEEMDPPENIDDIQSDLIFFQKRVPGHNKMEFESSLYEGHFLADQKEDDAFKLILKKKDENGDKSVMFTLTNLHQS

SEQ ID NO: 358 - protein, 157 aa, synthetic construct
NFGRLHWTTAVIRNINDQVLFVDKRQPVFEDMTDIDQSASEPQTRLIIYMYKDSEVRGLAVTLSVKDSKMSTLSAKNKIISFEEMDPPENIDDIQSDLIFFQKRVPGHNKMEFESSLYEGHFLADQKEDDAFKLILKKKDENGDKSVMFTLTNLHQS

SEQ ID NO: 359 - protein, 157 aa, synthetic construct
NFGRLHSTTAVIRNINDQVLFVDKRQPVFEDMTDIDQSASEPQTRLIIYMYKDSEVRGLAVTLSVKDSKMSTLSAKNKIISFEEMDPPENIDDIQSDLIFFQKRVPGHNKMEFESSLYEGHFLAVQKEDDAFKLILKKKDENGDKSVMFTLTNLHQS

SEQ ID NO: 360 - protein, 157 aa, synthetic construct
NFGRLHWTTAVIRNINDQVLFVDKRQPVFEDMTDIDQSASEPQTRLIIYMYKDSEVRGLAVTLSVKDSKMSTLSAKNKIISFEEMDPPENIDDIQSDLIFFQKRVPGHNKMEFESSLYEGHFLATQKEDDAFKLILKKKDENGDKSVMFTLTNLHQS

SEQ ID NO: 361 - protein, 157 aa, synthetic construct
NFGRLHSTTAVIRNINDQVLFVDKRQPVFEDMTDIDQSASEPQTRLIIYMYKDSEVRGLAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 362 - protein, 157 aa, synthetic construct
NFTRLHSTTAVIRNINDQVLFVDKRQPVFEDMTDIDQSASEPQTRLIIYMYKDSEVRGKAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 363 - protein, 157 aa, synthetic construct
NFVRLHSTTAVIRNINDQVLFVDKRQPVFEDMTDIDQSASEPQTRLIIYMYKDSEVRGKAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 364 - protein, 157 aa, synthetic construct
NFTRLKSTTAVIRNINDQVLFVDKRQPVFEDMTDIDQSASEPQTRLIIYMYKDSEVRGKAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 365 - protein, 157 aa, synthetic construct
NFVRLKSTTAVIRNINDQVLFVDKRQPVFEDMTDIDQSASEPQTRLIIYMYKDSEVRGKAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 366 - protein, 157 aa, synthetic construct
NFVRLHSTTAVIRNINDQVLFVDKRQPVFEDMTDIDQSASEPQTRLIIYMYKDSEVRGMAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 367 - protein, 157 aa, synthetic construct
NFVRLHSTTAVIRNINDQVLFVDKRQPVFEDMTDIDQSASEPQTRLIIYMYKDSEVRGRAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 368 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 369 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARSVPGHGRKTQFESSSYEGYFLACEKERDLFKLILKKEDELGDRSIMFTVQNED

SEQ ID NO: 370 - protein, 157 aa, synthetic construct
YFGKLESKLSVIRNLNDQVLFIDQGNRPLFEDMTDSDCRDNAPRTIFIISKYGDSGARGLAVTISVKCEKISTLSCENKIISFKEMNPPDNIKDTKSDIIFFERDVPGHSGKVQFESSSYEGYFLACEKERDLFKLILKKEDELGDRSIMFTVQNED

SEQ ID NO: 371 - protein, 157 aa, synthetic construct
NFGRLHCTTAVIRNINDQVLFVDKRQPVFEDMTDIDQSASEPQTRLIIYGYADSRVRGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 372 - protein, 157 aa, synthetic construct
HFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

(19) United States
(12) Patent Application Publication
(10) Pub. No.: US 2024/0182536 A1
(43) Pub. Date: Jun. 6, 2024

(54) INTERLEUKIN 18 VARIANTS

(71) Applicant: Mural Oncology, Inc., Waltham, MA (US)

(72) Inventors: Jean Chamoun, Waltham, MA (US); Jijun Dong, Weston, MA (US); Heather Flick, Waltham, MA (US); Pinar Gurel, Cambridge, MA (US); Yuan Hu, Belmont, MA (US); Mark Whitmore, Waltham, MA (US)

(21) Appl. No.: 18/496,582

(22) Filed: Oct. 27, 2023

Related U.S. Application Data
(60) Provisional application No. 63/424,701, filed on Nov. 11, 2022.

(51) Int. Cl. C07K 14/54; A61K 38/20; C07K 14/765

(52) U.S. Cl. CPC C07K 14/54; A61K 38/20; C07K 14/765; C07K 2319/30

(57) ABSTRACT

The disclosure provides engineered interleukin 18 (TL-18) polypeptides with reduced binding to IL-18 binding protein (IL-18BP) and maintained or improved binding to one or both of IL-18 Receptor alpha (IL-18Rα) and IL-18 Receptor beta (IL-18Rβ).

### RELATED APPLICATION

This application claims the benefit of U.S. Provisional Patent Application Ser. No. 63/424,701 filed Nov. 11, 2022, the entire disclosure of which is hereby incorporated herein by reference.

### BACKGROUND

Interleukin 18 (IL-18) is a potent pro-inflammatory cytokine expressed by a variety of immune cells to promote anti-viral and anti-cancer activity. However, the dysregulation or over-activity of IL-18 is implicated in several autoimmune disorders. These anti-viral, anti-cancer, and autoimmune-related aspects of IL-18 activity are mediated though the interaction between IL-18 and IL-18 Receptor alpha/IL-18 Receptor beta. Binding of IL-18 to IL-18Ralpha stimulates the recruitment of IL-18Rbeta, which in turn activates a signaling cascade within a target immune cell. The activity of IL-18 can be suppressed by extracellular interleukin 18 binding protein (IL-18BP) that binds soluble IL-18 with a higher affinity than IL-18Ralpha, thus prevents IL-18 binding to IL-18 receptor.

Attempts to use wild-type IL-18 as an anti-cancer and anti-viral therapeutic have been stymied by the repressive effect of IL-18BP. Moreover, IL-18 variants with eliminated IL-18BP binding are likely to have tolerability issues, as these variants will overstimulate the immune system leading to autoimmune issues and cytokine storm, in part due to IL-18-mediated expression of interferon gamma.

Accordingly, there exists a need in the art for IL-18 variants with reduced IL-18BP-mediated repression and maintained or enhanced IL-18 receptor binding.

### SUMMARY

In one aspect, the disclosure provides an interleukin 18 (IL-18) polypeptide comprising an amino acid substitution at position G3 of SEQ ID NO: 1.

In certain embodiments, the amino acid substitution comprises G3T, G3V, G3I, G3R, G3E, G3S, G3A, G3F, G3H, or G3K.

In certain embodiments, the IL-18 polypeptide further comprises one or more amino acid substitutions at positions L5, E6, S7, K8, L9, S10, R13, L15, 122, D23, R27, F30, T34, S36, D37, M38, D40, N41, M51, K53, Q56, S55, R58, G59, M60, T63, N87, N91, T95, Q103, R104, S105, P107, H109, D110, N111, M113, Q114, Q154, N155, E156, and D157.

In certain embodiments, the IL-18 polypeptide further comprises one or more amino acid substitutions at positions L5, E6, K8, M51, K53, Q56, G59, M60, T63, N91, T95, Q103, S105, D110, N111, M113, N155, E156, and D157.

In certain embodiments, the IL-18 polypeptide further comprises one or more amino acid substitutions at positions L5, E6, S7, K8, L9, S10, R13, L15, 122, D23, R27, F30, T34, S36, D37, M38, D40, N41, S55, R58, N87, R104, P107, H109, Q114, and Q154.

In certain embodiments, the amino acid substitution comprises L5I or L5W; E6K, E6R, E6Y, E6I, E6S, E6Q, E6L, E6M, E6T, E6A, E6D, E6G, E6H, or E6N; S7Y; K8R or K8H; L9V or L9G; S10C; R13S; L15I; I22M; D23G, D23H, or D23A; R27T or R27S; F30L; T34E, T34I, T34N, T34S, T34D, T34M, or T34P; S36F, S36P, S36V, S36N, S36Y, S36L, S36E, S36Q, S36W, or S36T; D37E or D37N; M38X, wherein X corresponds to a deletion of M38; D40A, D40I, D40E, D40H, D40Q, D40Y, D40T, D40F, D40R, D40K, or D40X, wherein X corresponds to a deletion of D40; N41G, N41D, N41M, N41T, N41Q, N41S, N41E, or N41I; M51K, M51R, M51Y, or M51F; K53A, K53S, K53T, K53H, K53N, K53T, K53G, K53S, K53D, K53Y, K53W, K53L, K53Q, K53V, K53R, or K53X, wherein X corresponds to a deletion of K53; S55M, S55R, S55Y, S55Q, S55D, S55K, S55I, S55V, S55L, S55A, S55E, S55N, S55P, S55G, S55W, or S55F; Q56E, Q56H, or Q56K; R58K; G59D; M60K, M60Q, or M60N; T63A, T63S, T63V, T63H, T63M, or T36I; N87D; N91R, N91L, N91M, N91E, N91A, N91S, N91V, N91W, N91I, N91Y, N91Q, N91F, N91K, N91H, N91T, N91E, N91G, or N91F; T95K or T95H; Q103A or Q103V; R104N, R014K, R104Y, R014S, R104G, R104Q, R104N, R104A, R104T, R104H, or R104P; S105R or S105F; P107K, P107S, P107Y, P107M, P107A, or P107N; H109Y, H109Q, H109I, H109F, or H109N; D110N, D110T, D110Q, D110H, or D110R; N111F, N111Q, or N111D; M113V, M113D, M113K, M113N, M113Q, M113S, M113A, M113E, or M113I; Q114R; Q154R; N155L, N155K, N155S, or N155Y; E156L; and D157E or D157R.

In certain embodiments, the IL-18 polypeptide comprises an amino acid substitution at position G3, E6, and K53 of SEQ ID NO: 1. In certain embodiments, the IL-18 polypeptide comprises an amino acid substitution of G3V, E6K, and K53A of SEQ ID NO: 1. In certain embodiments, the IL-18 polypeptide comprises an amino acid substitution of G3T, E6K, and K53A of SEQ ID NO: 1.

In certain embodiments, the IL-18 polypeptide comprises an amino acid substitution at position G3 and M60 of SEQ ID NO: 1. In certain embodiments, the IL-18 polypeptide comprises an amino acid substitution of G3V and M60K of SEQ ID NO: 1. In certain embodiments, the IL-18 polypeptide comprises an amino acid substitution of G3T and M60K of SEQ ID NO: 1.

In certain embodiments, the IL-18 polypeptide comprises an amino acid substitution at position G3, K53, and M60 of SEQ ID NO: 1. In certain embodiments, the IL-18 polypeptide comprises an amino acid substitution of G3V, K53A, and M60K of SEQ ID NO: 1. In certain embodiments, the IL-18 polypeptide comprises an amino acid substitution of G3V, K53S, and M60K of SEQ ID NO: 1. In certain embodiments, the IL-18 polypeptide comprises an amino acid substitution of G3T, K53A, and M60K of SEQ ID NO: 1. In certain embodiments, the IL-18 polypeptide comprises an amino acid substitution of G3T, K53S, and M60K of SEQ ID NO: 1.

In certain embodiments, the IL-18 polypeptide comprises an amino acid substitution at position G3, E6, K53, and M60 of SEQ ID NO: 1. In certain embodiments, the IL-18 polypeptide comprises an amino acid substitution of G3V, E6K, K53A, and M60K of SEQ ID NO: 1. In certain embodiments, the IL-18 polypeptide comprises an amino acid substitution of G3V, E6K, K53S, and M60K of SEQ ID NO: 1. In certain embodiments, the IL-18 polypeptide comprises an amino acid substitution of G3T, E6K, K53A, and M60K of SEQ ID NO: 1. In certain embodiments, the IL-18 polypeptide comprises an amino acid substitution of G3T, E6K, K53S, and M60K of SEQ ID NO: 1.

In certain embodiments, the IL-18 polypeptide comprises an amino acid substitution at position G3, M60, and D157 of SEQ ID NO: 1. In certain embodiments, the IL-18 polypeptide comprises an amino acid substitution of G3V, M60K, and D157E of SEQ ID NO: 1. In certain embodiments, the IL-18 polypeptide comprises an amino acid substitution of G3T, M60K, and D157E of SEQ ID NO: 1.

In certain embodiments, the IL-18 polypeptide comprises an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15, or an amino acid sequence with at least 80% identity to an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 2.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 3.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 4.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 5.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 6.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 7.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 8.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 9.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 10.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 11.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 12.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 13.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 14.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 15.

In certain embodiments, the IL-18 polypeptide comprises an amino acid sequence selected from the group consisting of SEQ ID NO: 2-SEQ ID NO: 311, or an amino acid sequence with at least 80% identity to an amino acid sequence selected from the group consisting of SEQ ID NO: 2-SEQ ID NO: 311.

In certain embodiments, the IL-18 polypeptide comprises a binding affinity to interleukin 18 binding protein (IL-18 BP) that is weaker than the binding affinity of an IL-18 polypeptide of SEQ ID NO: 1 to IL-18 BP.

In certain embodiments, the IL-18 polypeptide comprises a binding affinity to IL-18 BP of about 1 nM or weaker. In certain embodiments, the IL-18 polypeptide comprises a binding affinity to IL-18 BP of about 1 nM to about 1000 nM.

In certain embodiments, the IL-18 polypeptide retains binding affinity to interleukin 18 receptor alpha (IL-18Rα).

In certain embodiments, the binding affinity ratio of IL-18Rα binding affinity to IL-18BP binding affinity is less than the binding affinity ratio of an IL-18 polypeptide of SEQ ID NO: 1.

In certain embodiments, the binding affinity ratio of IL-18Rα binding affinity to IL-18BP binding affinity is about 0.01 to about 50. In certain embodiments, the binding affinity ratio of IL-18Rα binding affinity to IL-18BP binding affinity is about 0.1 to about 10.

In one aspect, the disclosure provides an interleukin 18 (IL-18) polypeptide comprising an amino acid substitution at position D157 of SEQ ID NO: 1.

In certain embodiments, the amino acid substitution comprises D157E or D157R.

In certain embodiments, the IL-18 polypeptide further comprises one or more amino acid substitutions at positions G3, L5, E6, S7, K8, L9, S10, R13, L15, 122, D23, R27, F30, T34, S36, D37, M38, D40, N41, M51, K53, Q56, S55, R58, G59, M60, T63, N87, N91, T95, Q103, R104, S105, P107, H109, D110, N111, M113, Q114, Q154, N155, and E156.

In certain embodiments, the IL-18 polypeptide further comprises one or more amino acid substitutions at positions G3, L5, E6, K8, M51, K53, Q56, G59, M60, T63, N91, T95, Q103, 5105, D110, N111, M113, N155, and E156.

In certain embodiments, the IL-18 polypeptide further comprises one or more amino acid substitutions at positions G3, L5, E6, S7, K8, L9, 510, R13, L15, 122, D23, R27, F30, T34, S36, D37, M38, D40, N41, S55, R58, N87, R104, P107, H109, Q114, and Q154.

In certain embodiments, the amino acid substitution comprises G3T, G3V, G3I, G3R, G3E, G3S, G3A, G3F, G3H, or G3K; L5I or L5W; E6K, E6R, E6Y, E61, E6S, E6Q, E6L, E6M, E6T, E6A, E6D, E6G, E6H, or E6N; S7Y; K8R or K8H; L9V or L9G; S10C; R13S; L15I; I22M; D23G, D23H, or D23A; R27T or R27S; F30L; T34E, T34I, T34N, T34S, T34D, T34M, or T34P; S36F, S36P, S36V, S36N, S36Y, S36L, S36E, S36Q, S36W, or S36T; D37E or D37N; M38X, wherein X corresponds to a deletion of M38; D40A, D40I, D40E, D40H, D40Q, D40Y, D40T, D40F, D40R, D40K, or D40X, wherein X corresponds to a deletion of D40; N41G, N41D, N41M, N41T, N41Q, N41S, N41E, or N41I; M51K, M51R, M51Y, or M51F; K53A, K53S, K53T, K53H, K53N, K53T, K53G, K53S, K53D, K53Y, K53W, K53L, K53Q, K53V, K53R, or K53X, wherein X corresponds to a deletion of K53; S55M, S55R, S55Y, S55Q, S55D, S55K, S55I, S55V, S55L, S55A, S55E, S55N, S55P, S55G, S55W, or S55F; Q56E, Q56H, or Q56K; R58K; G59D; M60K, M60Q, or M60N; T63A, T63S, T63V, T63H, T63M, or T36I; N87D; N91R, N91L, N91M, N91E, N91A, N91S, N91V, N91W, N91I, N91Y, N91Q, N91F, N91K, N91H, N91T, N91E, N91G, or N91F; T95K or T95H; Q103A or Q103V; R104N, R014K, R104Y, R014S, R104G, R104Q, R104N, R104A, R104T, R104H, or R104P; S105R or S105F; P107K, P107S, P107Y, P107M, P107A, or P107N; H109Y, H109Q, H109I, H109F, or H109N; D110N, D110T, D110Q, D110H, or D110R; N111F, N111Q, or N111D; M113V, M113D, M113K, M113N, M113Q, M113S, M113A, M113E, or M113I; Q114R; Q154R; N155L, N155K, N155S, or N155Y; and E156L.

In certain embodiments, the IL-18 polypeptide comprises an amino acid substitution at position G3, M60, and D157 of SEQ ID NO: 1. In certain embodiments, the IL-18 polypeptide comprises an amino acid substitution of G3V, M60K, and D157E of SEQ ID NO: 1. In certain embodiments, the IL-18 polypeptide comprises an amino acid substitution of G3T, M60K, and D157E of SEQ ID NO: 1.

In certain embodiments, the IL-18 polypeptide comprises an amino acid substitution at position M60 and D157 of SEQ ID NO: 1. In certain embodiments, the IL-18 polypeptide comprises an amino acid substitution of M60K and D157E of SEQ ID NO: 1.

In certain embodiments, the IL-18 polypeptide comprises a binding affinity to interleukin 18 binding protein (IL-18 BP) that is weaker than the binding affinity of an IL-18 polypeptide of SEQ ID NO: 1 to IL-18 BP.

In certain embodiments, the IL-18 polypeptide comprises a binding affinity to IL-18 BP of about 1 nM or weaker.

In certain embodiments, the IL-18 polypeptide comprises a binding affinity to IL-18 BP of about 1 nM to about 1000 nM.

In certain embodiments, the IL-18 polypeptide retains binding affinity to interleukin 18 receptor alpha (IL-18Rα).

In certain embodiments, the binding affinity ratio of IL-18Rα binding affinity to IL-18BP binding affinity is less than the binding affinity ratio of an IL-18 polypeptide of SEQ ID NO: 1.

In certain embodiments, the binding affinity ratio of IL-18Rα binding affinity to IL-18BP binding affinity is about 0.01 to about 50. In certain embodiments, the binding affinity ratio of IL-18Rα binding affinity to IL-18BP binding affinity is about 0.1 to about 10.

In certain embodiments, the IL-18 polypeptide comprising an amino acid sequence selected from the group consisting of SEQ ID NO: 14, SEQ ID NO: 15, SEQ ID NO: 114, SEQ ID NO: 115, SEQ ID NO: 119, SEQ ID NO: 142, SEQ ID NO: 143, SEQ ID NO: 144, SEQ ID NO: 146, SEQ ID NO: 147, SEQ ID NO: 148, SEQ ID NO: 150, SEQ ID NO: 151, and SEQ ID NO: 152, or an amino acid sequence with at least 80% identity to an amino acid sequence selected from the group consisting of SEQ ID NO: 14, SEQ ID NO: 15, SEQ ID NO: 114, SEQ ID NO: 115, SEQ ID NO: 119, SEQ ID NO: 142, SEQ ID NO: 143, SEQ ID NO: 144, SEQ ID NO: 146, SEQ ID NO: 147, SEQ ID NO: 148, SEQ ID NO: 150, SEQ ID NO: 151, and SEQ ID NO: 152.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 14.

In certain embodiments, the IL-18 polypeptide comprises an amino acid sequence with at least 8000 identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 15.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 114.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 115.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 119.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 142.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 143.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 144.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 146.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 147.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 148.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 150.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 151.

In certain embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99% or 100% identity) to an amino acid sequence selected from the group consisting of SEQ ID NO: 152.

In certain embodiments, the IL-18 polypeptide further comprises one or more substitutions of a cysteine amino acid with a non-cysteine amino acid.

In certain embodiments, the cysteine amino acid comprises one or more of C38, C68, C76, and C127 of SEQ ID NO: 1. In certain embodiments, the cysteine amino acid substitution comprises one or more of C38M, C68S, C76S, and C127S.

In certain embodiments, the IL-18 polypeptide further comprises a C38M, C68S, C76S, and C127S amino acid substitution.

In certain embodiments, the IL-18 polypeptide is PEGylated.

In certain embodiments, the IL-18 polypeptide is linked to an antibody Fe domain or serum albumin.

In certain embodiments, the IL-18 polypeptide is linked to an antibody Fe domain or serum albumin through an amino acid linker.

In certain embodiments, the amino acid linker comprises GGSGGGGSGGGSGGGGSGGGGSGGGSGG, GGGGSGGGGSGGGGS, GGGSGGGGSG GGSGGGGSGG, GGSGG, or GGS.

In certain embodiments, the antibody Fc domain comprises one or more mutations that alter effector function.

In certain embodiments, the antibody Fc domain comprises an IgG1 isotype comprising L234A/L235A mutations, according to EU numbering.

In certain embodiments, the IL-18 polypeptide further comprises a P329G mutation, according to EU numbering.

In certain embodiments, the antibody Fc domain comprises an IgG4 isotype comprising F234A/L235A mutations, according to EU numbering.

In certain embodiments, the antibody Fc domain comprises one or more heterodimerization mutations.

In certain embodiments, the antibody Fc domain comprises a first Fc polypeptide chain and a second Fc polypeptide chain.

In certain embodiments, the first Fc polypeptide chain comprises a T366S, L368A, and Y407V mutation, according to EU numbering, and the second Fc polypeptide chain comprises a T366W mutation, according to EU numbering.

In certain embodiments, the IL-18 polypeptide is linked to the first Fc polypeptide chain or the second Fc polypeptide chain.

In certain embodiments, the IL-18 polypeptide is linked to SEQ ID NO: 317. In certain embodiments, the IL-18 polypeptide is linked to SEQ ID NO: 318. In certain embodiments, the IL-18 polypeptide is linked to SEQ ID NO: 315. In certain embodiments, the IL-18 polypeptide is linked to SEQ ID NO: 316.

In certain embodiments, the IL-18 polypeptide is linked to any one or more of SEQ ID NO: 315, SEQ ID NO: 316, SEQ ID NO: 317, and SEQ ID NO: 318 via an amino acid linker. In certain embodiments, the amino acid linker comprises GGSGGGGSGGGSGGGGSGGGGSGGGSGG, GGGGSGGGGSGGGGS, GGGSGGGGSG GGSGGGGSGG, GGSGG, or GGS.

In certain embodiments, the IL-18 polypeptide comprises an N-terminal leader sequence.

In certain embodiments, the N-terminal leader sequence comprises MYRMQLLSCIALSLALVTNS, MAAEPVEDNCINFVAMKFIDNTLYFIAEDDENLESD, MAAEPVEDNCINFVAMKFIDNTLYFIAEDDENLESD, or MAAMSEDSCVNFKEMMFIDNTLYFIPEENGDLESD.

In one aspect, the disclosure provides a pharmaceutical composition comprising the IL-18 polypeptide described above, and a pharmaceutically acceptable carrier or diluent.

In one aspect, the disclosure provides a polynucleotide sequence that encodes the IL-18 polypeptide described above.

In one aspect, the disclosure provides an expression vector comprising the polynucleotide sequence described above.

In one aspect, the disclosure provides a host cell comprising the expression vector described above.

In one aspect, the disclosure provides a method of producing the IL-18 polypeptide described above, comprising culturing the host cell described above under conditions to express the IL-18 polypeptide.

In certain embodiments, the method further comprises isolating the IL-18 polypeptide from the host cell.

In one aspect, the disclosure provides a method of treating cancer in a patient in need thereof comprising administering to the patient a therapeutically effective amount of the pharmaceutical composition described above or the IL-18 polypeptide described above.

### BRIEF DESCRIPTION OF THE DRAWINGS

FIG. 1A-FIG. 1B depict relative activity of select IL-18 variants in a HEK Blue assay. IL-18 variants were tested without co-incubation with human IL-18BP (FIG. 1A) and with 300 nM human IL-18BP (FIG. 1B).

FIG. 2 depicts relative activity of select IL-18 variants in the presence of titrated human IL-18BP.

FIG. 3A-FIG. 3B depict relative activity of select IL-18 variants in a HEK Blue assay. IL-18 variants were tested with co-incubation with human IL-18BP at 50 nM (FIG. 3A) and at 300 nM (FIG. 3B).

FIG. 4A-FIG. 4B depict IFN gamma expression of select IL-18 variants in a human PBMC stimulated IFN gamma assay. IL-18 variants were tested with co-incubation without human IL-18BP (FIG. 4A) and with human IL-18BP (FIG. 4B).

FIG. 5A-FIG. 5B depict IFN gamma expression of select IL-18 variants in a human PBMC stimulated IFN gamma assay. IL-18 variants were tested with co-incubation without human IL-18BP (FIG. 5A) and with human IL-18BP (FIG. 5B).

FIG. 6A-FIG. 6F depict IFN gamma expression of IL-18 variants with at least the M60K substitution in a human PBMC stimulated IFN gamma assay. IL-18 variants were tested with co-incubation with or without 300 nM IL-18BP.

FIG. 7 depicts IFN gamma expression of select IL-18 variants with the K53S or K53A substitution in a human PBMC stimulated IFN gamma assay.

FIG. 8 depicts relative activity of select IL-18 variants with the K53S or K53A substitution in a BEK Blue assay with or without 50 nM IL-18BP.

FIG. 9A-FIG. 9B depict IFN gamma expression of select IL-18 variants in a human PBMC stimulated IFN gamma assay. IL-18 variants were tested with co-incubation with human IL-18BP (FIG. 9A) and mouse IL-18BP (FIG. 9B).

FIG. 10 depicts IFN gamma expression of select IL-18 variants with the K53S or K53A substitution in a human PBMC stimulated IFN gamma assay.

FIG. 11 depicts relative activity of select IL-18 variants with the K53S or K53A substitution in a BEK Blue assay with or without 50 nM IL-18BP.

FIG. 12A-FIG. 12B depict IFN gamma expression of select IL-18 variants in a human PBMC stimulated IFN gamma assay (FIG. 12A) and relative activity of select IL-18 variants in a BEK Blue assay with or without 50 nM IL-18BP (FIG. 12B).

FIG. 13A-FIG. 13B depict IFN gamma expression of select IL-18 variants in an exhausted CD8+ T cell assay with WT IL-18 and an IL-18 variant comparator (FIG. 13A) and the select IL-18 variants (FIG. 13B).

FIG. 14A-FIG. 14B depict relative activity of select IL-18 variants linked to various polypeptides in a HEK Blue assay. IL-18 variants were tested without IL-18BP (FIG. 14A) and with IL-18BP at 300 nM (FIG. 14B).

FIG. 15A-FIG. 15B depict relative activity of select IL-18 variants in a HEK Blue assay. IL-18 variants were tested without IL-18BP (FIG. 15A) and with IL-18BP at 300 nM (FIG. 15B).

FIG. 16 depicts the cytotoxic activity of select IL-18 variants in a A549 cell/PBMC killing assay as measured by the % of A549 nuclei.

FIG. 17 depicts the cytotoxic activity of select IL-18 variants in a A549 cell/PBMC killing assay as measured by the % of A549 nuclei.

FIG. 18A-FIG. 18H depict IFN gamma expression of select IL-18 variants in a human PBMC stimulated IFN gamma assay.

### DETAILED DESCRIPTION

Provided herein are IL-18 variants with reduced IL-18BP binding and maintained or enhanced IL-18 receptor binding (i.e., IL-18Rα and/or IL-18Rβ). The IL-18 variant disclosed herein possess robust activity as measured in part by interferon gamma expression and PBMC killing assays. The instant disclosure described the surprising discovery that numerous amino acid mutations (e.g., substitutions and deletions) that can be made to the wild-type IL-18 polypeptide to reduce IL-18BP binding and maintain or enhance IL-18 receptor binding. Said mutations maintain or enhance the potency of IL-18 relative to wild-type IL-18.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the invention may be more readily understood, certain terms are first defined.

### IL-18 Cytokine Variants

As used herein, an “IL-18 cytokine variant” or “IL-18 variant” or “interleukin 18 variant” or “engineered IL-18 polypeptide” refers to an IL-18 polypeptide that comprises one or more amino acid mutations (e.g., substitutions) relative to the wild-type (WT) IL-18 amino acid sequence (SEQ ID NO: 1) that alters the activity of the IL-18 polypeptide. The WT IL-18 amino acid sequence of SEQ ID NO: 1 is recited below:

Wild-type IL-18:
(SEQ ID NO: 1)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGHDNKMQFESSSYEGYFLACEKERDLFKLILKKEDELGDRSIM
FTVQNED

IL-18 is a pro-inflammatory cytokine belonging to the IL-1 superfamily of cytokines. Upon binding to the IL-18 receptor, IL-18 mediates the expression of other pro-inflammatory markers, including, but not limited to, interferon gamma. The IL-18 receptor is composed of IL-18Rα, which binds mature IL-18 with low affinity, and the co-receptor IL-18Rβ. IL-18 binds the ligand receptor IL-18Rα, inducing the recruitment of IL-18Rβ to form a high affinity complex (Kaplanski. Immunol Rev. 2018. 281(1): 138-153).

The activity of IL-18 can be suppressed by extracellular interleukin 18 binding protein (IL-18BP) that binds soluble IL-18 with a higher affinity than IL-18Rα thus prevents IL-18 binding to IL-18 receptor (Kaplanski, supra).

The IL-18 gene, similar to other IL-1 family members, lacks a signal peptide for secretion out of the cell. Furthermore, IL-18 is produced as a biologically inactive precursor. The IL-18 gene encodes for a 193 amino acids precursor, first synthesized as an inactive 24 kDa precursor with no signal peptide, which accumulates in cell cytoplasm. The IL-18 precursor is processed intracellularly by caspase 1 in the NLRP3 inflammasome into its mature biologically active molecule of 18 kDa.

Provided herein are IL-18 variants with reduced IL-18BP binding and maintained or enhanced IL-18 receptor binding (i.e., IL-18Rα and/or IL-18Rβ). The IL-18 variant disclosed herein possess robust activity as measured in part by interferon gamma expression and PBMC killing assays.

### G3-Based IL-18 Variants:

Select IL-18 variants are based on an amino acid substitution at position G3 of wild-type IL-18 (SEQ ID NO: 1). Additional amino acid substitutions or deletions can be made to the wild-type IL-18 to further alter the activity of IL-18.

In one aspect, the disclosure provides an interleukin 18 (IL-18) polypeptide comprising an amino acid substitution at position G3 of SEQ ID NO: 1. In certain embodiments, the amino acid substitution comprises G3T, G3V, G3I, G3R, G3E, G3S, G3A, G3F, G3H, or G3K.

In certain embodiments, the IL-18 polypeptide further comprises one or more amino acid substitutions at positions L5, E6, S7, K8, L9, S10, R13, L15, 122, D23, R27, F30, T34, S36, D37, M38, D40, N41, M51, K53, Q56, S55, R58, G59, M60, T63, N87, N91, T95, Q103, R104, S105, P107, H109, D110, N111, M113, Q114, Q154, N155, E156, and D157.

In certain embodiments, the IL-18 polypeptide further comprises one or more amino acid substitutions at positions L5, E6, K8, M51, K53, Q56, G59, M60, T63, N91, T95, Q103, S105, D110, N111, M113, N155, E156, and D157.

In certain embodiments, the IL-18 polypeptide further comprises one or more amino acid substitutions at positions L5, E6, S7, K8, L9, 510, R13, L15, 122, D23, R27, F30, T34, S36, D37, M38, D40, N41, S55, R58, N87, R104, P107, H109, Q114, and Q154.

In certain embodiments, the amino acid substitution comprises: L5I or L5W; E6K, E6R, E6Y, E61, E6S, E6Q, E6L, E6M, E6T, E6A, E6D, E6G, E6H, or E6N; S7Y; K8R or K8H; L9V or L9G; S10C; R13S; L15I; I22M; D23G, D23H, or D23A; R27T or R27S; F30L; T34E, T34I, T34N, T34S, T34D, T34M, or T34P; S36F, S36P, S36V, S36N, S36Y, S36L, S36E, S36Q, S36W, or S36T; D37E or D37N; M38X, wherein X corresponds to a deletion of M38; D40A, D40I, D40E, D40H, D40Q, D40Y, D40T, D40F, D40R, D40K, or D40X, wherein X corresponds to a deletion of D40; N41G, N41D, N41M, N41T, N41Q, N41S, N41E, or N41I; M51K, M51R, M51Y, or M51F; K53A, K53S, K53T, K53H, K53N, K53T, K53G, K53S, K53D, K53Y, K53W, K53L, K53Q, K53V, K53R, or K53X, wherein X corresponds to a deletion of K53; S55M, S55R, S55Y, S55Q, S55D, S55K, S55I, S55V, S55L, S55A, S55E, S55N, S55P, S55G, S55W, or S55F; Q56E, Q56H, or Q56K; R58K; G59D; M60K, M60Q, or M60N; T63A, T63S, T63V, T63H, T63M, or T36I; N87D; N91R, N91L, N91M, N91E, N91A, N91S, N91V, N91W, N91I, N91Y, N91Q, N91F, N91K, N91H, N91T, N91E, N91G, or N91F; T95K or T95H; Q103A or Q103V; R104N, R014K, R104Y, R014S, R104G, R104Q, R104N, R104A, R104T, R104H, or R104P; S105R or S105F; P107K, P107S, P107Y, P107M, P107A, or P107N; H109Y, H109Q, H109I, H109F, or H109N; D110N, D110T, D110Q, D110H, or D110R; N111F, N111Q, or N111D; M113V, M113D, M113K, M113N, M113Q, M113S, M113A, M113E, or M113I; Q114R; Q154R; N155L, N155K, N155S, or N155Y; E156L; and D157E or D157R.

In certain embodiments, the IL-18 polypeptide comprises an amino acid substitution at position G3, E6, and K53 of SEQ ID NO: 1. In certain embodiments, the IL-18 polypeptide comprises an amino acid substitution of G3V, E6K, and K53A of SEQ ID NO: 1. In certain embodiments, the IL-18 polypeptide comprises an amino acid substitution of G3T, E6K, and K53A of SEQ ID NO: 1.

In certain embodiments, the IL-18 polypeptide comprises an amino acid substitution at position G3 and M60 of SEQ ID NO: 1. In certain embodiments, the IL-18 polypeptide comprises an amino acid substitution of G3V and M60K of SEQ ID NO: 1. In certain embodiments, the IL-18 polypeptide comprises an amino acid substitution of G3T and M60K of SEQ ID NO: 1.

In certain embodiments, the IL-18 polypeptide comprises an amino acid substitution at position G3, K53, and M60 of SEQ ID NO: 1. In certain embodiments, the IL-18 polypeptide comprises an amino acid substitution of G3V, K53A, and M60K of SEQ ID NO: 1. In certain embodiments, the IL-18 polypeptide comprises an amino acid substitution of G3V, K53S, and M60K of SEQ ID NO: 1. In certain embodiments, the IL-18 polypeptide comprises an amino acid substitution of G3T, K53A, and M60K of SEQ ID NO: 1. In certain embodiments, the IL-18 polypeptide comprises an amino acid substitution of G3T, K53S, and M60K of SEQ ID NO: 1.

In certain embodiments, the IL-18 polypeptide comprises an amino acid substitution at position G3, E6, K53, and M60 of SEQ ID NO: 1. In certain embodiments, the IL-18 polypeptide comprises an amino acid substitution of G3V, E6K, K53A, and M60K of SEQ ID NO: 1. In certain embodiments, the IL-18 polypeptide comprises an amino acid substitution of G3V, E6K, K53S, and M60K of SEQ ID NO: 1. In certain embodiments, the IL-18 polypeptide comprises an amino acid substitution of G3T, E6K, K53A, and M60K of SEQ ID NO: 1. In certain embodiments, the IL-18 polypeptide comprises an amino acid substitution of G3T, E6K, K53S, and M60K of SEQ ID NO: 1.

In certain embodiments, the IL-18 polypeptide comprises an amino acid substitution at position G3, M60, and D157 of SEQ ID NO: 1. In certain embodiments, the IL-18 polypeptide comprises an amino acid substitution of G3V, M60K, and D157E of SEQ ID NO: 1. In certain embodiments, the IL-18 polypeptide comprises an amino acid substitution of G3T, M60K, and D157E of SEQ ID NO: 1.

In certain embodiments, the IL-18 polypeptide comprises an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

In other embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

In certain embodiments, the IL-18 polypeptide comprises a binding affinity to interleukin 18 binding protein (IL-18 BP) that is weaker than the binding affinity of an IL-18 polypeptide of SEQ ID NO: 1 to IL-18 BP.

In certain embodiments, the IL-18 polypeptide comprises a binding affinity to IL-18 BP of about 1 nM or weaker (e.g., about 1 nM, about 10 nM, about 50 nM, about 100 nM, about 200 nM, about 300 nM, about 400 nM, about 500 nM, about 600 nM, about 700 nM, about 800 nM, about 900 nM, about 1000 nM, about 2000 nM, about 3000 nM, about 4000 nM, about 5000 nM, about 6000 nM, about 7000 nM, about 8000 nM, about 9000 nM, about 10000 nM, or weaker). In certain embodiments, the IL-18 polypeptide comprises a binding affinity to IL-18 BP of about 1 nM to about 10000 nM. In certain embodiments, the IL-18 polypeptide comprises a binding affinity to IL-18 BP of about 1 nM to about 10000 nM in the presence of about 1 nM to about 10000 nM of IL-18BP.

In certain embodiments, the IL-18 polypeptide retains binding affinity to interleukin 18 receptor alpha (IL-18Rα). In certain embodiments, the IL-18 polypeptide comprises a binding affinity to IL-18Rα of about 1 nM to about 10000 nM (e.g., about 1 nM, about 10 nM, about 50 nM, about 100 nM, about 200 nM, about 300 nM, about 400 nM, about 500 nM, about 600 nM, about 700 nM, about 800 nM, about 900 nM, about 1000 nM, about 2000 nM, about 3000 nM, about 4000 nM, about 5000 nM, about 6000 nM, about 7000 nM, about 8000 nM, about 9000 nM, or about 10000 nM). In certain embodiments, the IL-18 polypeptide comprises a binding affinity to IL-18Rα of about 50 nM to about 500 nM.

In certain embodiments, the binding affinity ratio of IL-18Rα binding affinity to IL-18BP binding affinity is less than the binding affinity ratio of an IL-18 polypeptide of SEQ ID NO: 1. In certain embodiments, the binding affinity ratio of IL-18Rα binding affinity to IL-18BP binding affinity is about 0.01 to about 50 (e.g., about 0.01, about 0.1, about 0.5, about 1, about 5, about 10, about 15, about 20, about 25, about 30, about 35, about 40, about 45, or about 50). In certain embodiments, the binding affinity ratio of IL-18Rα binding affinity to IL-18BP binding affinity is about 0.1 to about 10.

The above recited binding affinities (i.e., IL-18 to IL-18Rα or IL-18 to IL-18BP) may be readily determined by any means known in the art, including but not limited to, Octet Biolayer Interferometry as described in Example 1.

### D157-Based IL-18 Variants:

Select IL-18 variants are based on an amino acid substitution at position D157 of wild-type IL-18 (SEQ ID NO: 1). Additional amino acid substitutions or deletions can be made to the wild-type IL-18 to further alter the activity of IL-18.

In one aspect, the disclosure provides an interleukin 18 (IL-18) polypeptide comprising an amino acid substitution at position D157 of SEQ ID NO: 1. In certain embodiments, the amino acid substitution comprises D157E or D157R.

In certain embodiments, the IL-18 polypeptide further comprises one or more amino acid substitutions at positions G3, L5, E6, S7, K8, L9, S10, R13, L15, 122, D23, R27, F30, T34, S36, D37, M38, D40, N41, M51, K53, Q56, S55, R58, G59, M60, T63, N87, N91, T95, Q103, R104, S105, P107, H109, D110, N111, M113, Q114, Q154, N155, and E156.

In certain embodiments, the IL-18 polypeptide further comprises one or more amino acid substitutions at positions G3, L5, E6, K8, M51, K53, Q56, G59, M60, T63, N91, T95, Q103, S105, D110, N111, M113, N155, and E156.

In certain embodiments, the IL-18 polypeptide further comprises one or more amino acid substitutions at positions G3, L5, E6, S7, K8, L9, S10, R13, L15, 122, D23, R27, F30, T34, S36, D37, M38, D40, N41, S55, R58, N87, R104, P107, H109, Q114, and Q154.

In certain embodiments, the amino acid substitution comprises: G3T, G3V, G3I, G3R, G3E, G3S, G3A, G3F, G3H, or G3K; L5I or L5W; E6K, E6R, E6Y, E6I, E6S, E6Q, E6L, E6M, E6T, E6A, E6D, E6G, E6H, or E6N; S7Y; K8R or K8H; L9V or L9G; S10C; R13S; L15I; I22M; D23G, D23H, or D23A; R27T or R27S; F30L; T34E, T34I, T34N, T34S, T34D, T34M, or T34P; S36F, S36P, S36V, S36N, S36Y, S36L, S36E, S36Q, S36W, or S36T; D37E or D37N; M38X, wherein X corresponds to a deletion of M38; D40A, D40I, D40E, D40H, D40Q, D40Y, D40T, D40F, D40R, D40K, or D40X, wherein X corresponds to a deletion of D40; N41G, N41D, N41M, N41T, N41Q, N41S, N41E, or N41I; M51K, M51R, M51Y, or M51F; K53A, K53S, K53T, K53H, K53N, K53T, K53G, K53S, K53D, K53Y, K53W, K53L, K53Q, K53V, K53R, or K53X, wherein X corresponds to a deletion of K53; S55M, S55R, S55Y, S55Q, S55D, S55K, S55I, S55V, S55L, S55A, S55E, S55N, S55P, S55G, S55W, or S55F; Q56E, Q56H, or Q56K; R58K; G59D; M60K, M60Q, or M60N; T63A, T63S, T63V, T63H, T63M, or T36I; N87D; N91R, N91L, N91M, N91E, N91A, N91S, N91V, N91W, N91I, N91Y, N91Q, N91F, N91K, N91H, N91T, N91E, N91G, or N91F; T95K or T95H; Q103A or Q103V; R104N, R014K, R104Y, R014S, R104G, R104Q, R104N, R104A, R104T, R104H, or R104P; S105R or S105F; P107K, P107S, P107Y, P107M, P107A, or P107N; H109Y, H109Q, H109I, H109F, or H109N; D110N, D110T, D110Q, D110H, or D110R; N111F, N111Q, or N111D; M113V, M113D, M113K, M113N, M113Q, M113S, M113A, M113E, or M113I; Q114R; Q154R; N155L, N155K, N155S, or N155Y; and E156L.

In certain embodiments, the IL-18 polypeptide comprises an amino acid substitution at position G3, M60, and D157 of SEQ ID NO: 1. In certain embodiments, the IL-18 polypeptide comprises an amino acid substitution of G3V, M60K, and D157E of SEQ ID NO: 1. In certain embodiments, the IL-18 polypeptide comprises an amino acid substitution of G3T, M60K, and D157E of SEQ ID NO: 1.

In certain embodiments, the IL-18 polypeptide comprises an amino acid substitution at position M60 and D157 of SEQ ID NO: 1. In certain embodiments, the IL-18 polypeptide comprises an amino acid substitution of M60K and D157E of SEQ ID NO: 1.

In certain embodiments, the IL-18 polypeptide comprises an amino acid sequence of SEQ ID NO: 14 or SEQ ID NO: 15.

In other embodiments, the IL-18 polypeptide comprises an amino acid sequence with at least 80% identity (i.e., 80%, 81%, 82%, 83%, 84%, 85%, 86%, 87%, 88%, 89%, 90%, 91%, 92%, 93%, 94%, 95%, 96%, 97%, 98%, 99%, or 100% identity) to an amino acid sequence of SEQ ID NO: 14 or SEQ ID NO: 15.

In certain embodiments, the IL-18 polypeptide comprises a binding affinity to interleukin 18 binding protein (IL-18 BP) that is weaker than the binding affinity of an IL-18 polypeptide of SEQ ID NO: 1 to IL-18 BP.

In certain embodiments, the IL-18 polypeptide comprises a binding affinity to IL-18 BP of about 1 nM or weaker (e.g., about 1 nM, about 10 nM, about 50 nM, about 100 nM, about 200 nM, about 300 nM, about 400 nM, about 500 nM, about 600 nM, about 700 nM, about 800 nM, about 900 nM, about 1000 nM, about 2000 nM, about 3000 nM, about 4000 nM, about 5000 nM, about 6000 nM, about 7000 nM, about 8000 nM, about 9000 nM, about 10000 nM, or weaker). In certain embodiments, the IL-18 polypeptide comprises a binding affinity to IL-18 BP of about 1 nM to about 10000 nM. In certain embodiments, the IL-18 polypeptide comprises a binding affinity to IL-18 BP of about 1 nM to about 10000 nM in the presence of about 1 nM to about 10000 nM of IL-18BP.

In certain embodiments, the IL-18 polypeptide retains binding affinity to interleukin 18 receptor alpha (IL-18Rα). In certain embodiments, the IL-18 polypeptide comprises a binding affinity to IL-18Rα of about 1 nM to about 10000 nM (e.g., about 1 nM, about 10 nM, about 50 nM, about 100 nM, about 200 nM, about 300 nM, about 400 nM, about 500 nM, about 600 nM, about 700 nM, about 800 nM, about 900 nM, about 1000 nM, about 2000 nM, about 3000 nM, about 4000 nM, about 5000 nM, about 6000 nM, about 7000 nM, about 8000 nM, about 9000 nM, or about 10000 nM). In certain embodiments, the IL-18 polypeptide comprises a binding affinity to IL-18Rα of about 50 nM to about 500 nM.

In certain embodiments, the binding affinity ratio of IL-18Rα binding affinity to IL-18BP binding affinity is less than the binding affinity ratio of an IL-18 polypeptide of SEQ ID NO: 1. In certain embodiments, the binding affinity ratio of IL-18Rα binding affinity to IL-18BP binding affinity is about 0.01 to about 50 (e.g., about 0.01, about 0.1, about 0.5, about 1, about 5, about 10, about 15, about 20, about 25, about 30, about 35, about 40, about 45, or about 50). In certain embodiments, the binding affinity ratio of IL-18Rα binding affinity to IL-18BP binding affinity is about 0.1 to about 10.

The above recited binding affinities (i.e., IL-18 to IL-18Rα or IL-18 to IL-18BP) may be readily determined by any means known in the art, including but not limited to, Octet Biolayer Interferometry as described in Example 1.

### Cysteine-Substituted IL-18 Variants:

The IL-18 variants described herein (e.g., the G3 substituted variants and D157 variants described above) may further comprise one or more substitutions of a cysteine amino acid with a non-cysteine amino acid. In certain contexts, the cysteine amino acids may decrease stability. It is therefore advantageous to substitute certain cysteine amino acids to non-cysteine amino acids, while retaining the activity of the IL-18 variant. The cysteine substitutions described herein may be employed in the IL-18 variants are being expressed in mammalian cells (e.g., CHO or HEK cells). However, the IL-18 variants may lack the cysteine substitutions if the IL-18 variants are being expressed in a prokaryotic cell (e.g., E. coli).

In certain embodiments, the cysteine amino acid comprises one or more of C38, C68, C76, and C127 of SEQ ID NO: 1. In certain embodiments, the cysteine amino acid substitution comprises one or more of C38M, C68S, C76S, and C127S.

In certain embodiments, the IL-18 polypeptide further comprises a C38M, C68S, C76S, and C127S amino acid substitution.

### IL-18 Variant Fusion Proteins:

The IL-18 variants described herein (e.g., the G3 substituted variants and D157 variants described above) may be further linked or conjugated to one or more functional moieties. The functional moiety may confer one or more additional properties onto the IL-18 variant, such as increased or decreased serum half-life or reduced immunogenicity.

In certain embodiments, the functional moiety is ethylene glycol, including polyethylene glycol (PEG). Accordingly, in certain embodiments, the IL-18 polypeptide is PEGylated.

In certain embodiments, the functional moiety is an antibody Fc domain. In certain embodiments, the antibody Fc domain is an IgG1 or IgG4 isotype. The antibody Fc domain comprises a first Fc polypeptide chain and a second Fc polypeptide chain which dimerize to form the Fc domain.

In certain embodiments, the antibody Fc domain comprises one or more mutations that alter effector function. Antibody Fc domain effector functions are often mediated through an interaction between the Fc domain and an Fc receptor (e.g., FcR gamma). The altered effector function may be one or both of antibody dependent cellular cytotoxicity (ADCC) and complement dependent cytotoxicity (CDC).

In certain embodiments, the antibody Fc domain comprises an IgG1 isotype comprising L234A/L235A mutations, according to EU numbering. In certain embodiments, the antibody Fc domain further comprises a P329G mutation, according to EU numbering.

In certain embodiments, the antibody Fe domain comprises an IgG4 isotype comprising F234A/L235A mutations, according to EU numbering.

When the first and second Fc polypeptide chains differ, it may be important to employ one or more heterodimerization mutations in one or both Fc polypeptide chains to facilitate heterodimerization of the two different chains. Accordingly, in certain embodiments, the antibody Fc domain comprises one or more heterodimerization mutations. In certain embodiments, the first Fc polypeptide chain comprises a T366S, L368A, and Y407V mutation, according to EU numbering, and the second Fc polypeptide chain comprises a T366W mutation, according to EU numbering. In other embodiments, the first Fc polypeptide chain comprises a T366W mutation, according to EU numbering, and the second Fc polypeptide chain comprises a T366S, L368A, and Y407V mutation, according to EU numbering.

In certain embodiments, the IL-18 polypeptide described herein is linked to the first Fc polypeptide chain or the second Fc polypeptide chain. Accordingly in the context of heterodimerization mutation-containing Fc domains, only one polypeptide chain (i.e., the first Fc polypeptide chain or the second Fc polypeptide chain) will be linked to the IL-18 polypeptide.

In certain embodiments, the functional moiety is serum albumin. In certain embodiments, the serum albumin is human serum albumin (HSA). An exemplary HSA amino acid sequence is recited below:

(SEQ ID NO: 312)
RGVFRRDAHKSEVAHRFKDLGEENFKALVLIAFAQYLQQCPFEDHVKLVN
EVTEFAKTCVADESAENCDKSLHTLFGDKLCTVATLRETYGEMADCCAKQ
EPERNECFLQHKDDNPNLPRLVRPEVDVMCTAFHDNEETFLKKYLYEIAR
RHPYFYAPELLFFAKRYKAAFTECCQAADKAACLLPKLDELRDEGKASSA
KQGLKCASLQKFGERAFKAWAVARLSQRFPKAEFAEVSKLVTDLTKVHTE
CCHGDLLECADDRADLAKYICENQDSISSKLKECCEKPLLEKSHCIAEVE
NDEMPADLPSLAADFVGSKDVCKNYAEAKDVFLGMFLYEYARRHPDYSVV
LLLRLAKTYETTLEKCCAAADPHECYAKVFDEFKPLVEEPQNLIKQNCEL
FEQLGEYKFQNALLVRYTKKVPQVSTPTLVEVSRNLGKVGSKCCKHPEAK
RMPCAEDCLSVFLNQLCVLHEKTPVSDRVTKCCTESLVNGRPCFSALEVD
ETYVPKEFNAETFTFHADICTLSEKERQIKKQTALVELVKHKPKATKEQL
KAVMDDFAAFVEKCCKADDKETCFAEEGKKLVAASQAALGL.

Exemplary antibody Fe domains that may be linked to the IL-18 variants of the disclosure are provided below.

| | Human/ | |
|---|---|---|
| Description | Mouse | Sequence |
| IgG1 CH1- | human | ASTKGPSVFPLAPSSKSTSGGTAALGCLVKD |
| hinge-CH2- | | YFPEPVTVSWNSGALTSGVHTFPAVLQSSGL |
| CH3 (LALA) | | YSLSSVVTVPSSSLGTQTYICNVNHKPSNTK |
| | | VDKKVEPKSCDKTHTCPPCPAPEAAGGPSVF |
| | | LFPPKPKDTLMISRTPEVTCVVVDVSHEDPE |
| | | VKFNWYVDGVEVHNAKTKPREEQYNSTYRVV |
| | | SVLTVLHQDWLNGKEYKCKVSNKALPAPIEK |
| | | TISKAKGQPREPQVYTLPPSRDELTKNQVSL |
| | | TCLVKGFYPSDIAVEWESNGQPENNYKTTPP |
| | | VLDSDGSFFLYSKLTVDKSRWQQGNVFSCSV |
| | | MHEALHNHYTQKSLSLSPGK |
| | | (SEQ ID NO: 313) |
| mIgG2a hinge- | mouse | EPRGPTIKPCPPCKCPAPNLLGGPSVFIFPP |
| CH2-CH3 | | KIKDVLMISLSPIVTCVVVDVSEDDPDVQIS |
| | | WFVNNVEVHTAQTQTHREDYNSTLRVVSALP |
| | | IQHQDWMSGKEFKCKVNNKDLPAPIERTISK |
| | | PKGSVRAPQVYVLPPPEEEMTKKQVTLTCMV |
| | | TDFMPEDIYVEWTNNGKTELNYKNTEPVLDS |
| | | DGSYFMYSKLRVEKKNWVERNSYSCSVVHEG |
| | | LHNHHTTKSFSRTPGK |
| | | (SEQ ID NO: 314) |
| Knob (Human | human | EPKSSDKTHTCPPCPAPEAAGGPSVFLFPPK |
| IgG1 CH2-CH3 | | PKDTLMISRTPEVTCVVVDVSHEDPEVKFNW |
| T366W, LALA- | | YVDGVEVHNAKTKPREEQYNSTYRVVSVLTV |
| PG) | | LHQDWLNGKEYKCKVSNKALGAPIEKTISKA |
| | | KGQPREPQVCTLPPSREEMTKNQVSLWCLVK |
| | | GFYPSDIAVEWESNGQPENNYKTTPPVLDSD |
| | | GSFFLYSKLTVDKSRWQQGNVFSCSVMHEAL |
| | | HNHYTQKSLSLSPGK |
| | | (SEQ ID NO: 315) |
| Hole (Human | human | EPKSSDKTHTCPPCPAPEAAGGPSVFLFPPK |
| IgG1 CH2-CH3, | | PKDTLMISRTPEVTCVVVDVSHEDPEVKENW |
| T366S, L368A, | | YVDGVEVHNAKTKPREEQYNSTYRVVSVLTV |
| Y407V, LALA- | | LHQDWLNGKEYKCKVSNKALGAPIEKTISKA |
| PG, HR YF | | KGQPREPQVYTLPPCREEMTKNQVSLSCAVK |
| proA binding | | GFYPSDIAVEWESNGQPENNYKTTPPVLDSD |
| null) | | GSFFLVSKLTVDKSRWQQGNVFSCSVMHEAL |
| | | HNRFTQKSLSLSPGK |
| | | (SEQ ID NO: 316) |
| Human Fc Knob | human | EPKSSDKTHTCPPCPAPEAAGGPSVFLFPPK |
| (hlgG1-CH2- | | PKDTLMISRTPEVTCVVVDVSHEDPEVKFNW |
| CH3, T366W, | | YVDGVEVHNAKTKPREEQYNSTYRVVSVLTV |
| LALA, HR | | LHQDWLNGKEYKCKVSNKALPAPIEKTISKA |
| YF proA | | KGQPREPQVCTLPPSREEMTKNQVSLWCLVK |
| binding null) | | GFYPSDIAVEWESNGQPENNYKTTPPVLDSD |
| | | GSFFLYSKLTVDKSRWQQGNVFSCSVMHEAL |
| | | HNRFTQKSLSLSPGK |
| | | (SEQ ID NO: 317) |
| Human Fc Hole | human | EPKSSDKTHTCPPCPAPEAAGGPSVFLFPPK |
| (hIgG1-CH2- | | PKDTLMISRTPEVTCVVVDVSHEDPEVKFNW |
| CH3, T366S, | | YVDGVEVHNAKTKPREEQYNSTYRVVSVLTV |
| L368A, Y407, | | LHQDWLNGKEYKCKVSNKALPAPIEKTISKA |
| LALA) | | KGQPREPQVYTLPPCREEMTKNQVSLSCAVK |
| | | GFYPSDIAVEWESNGQPENNYKTTPPVLDSD |
| | | GSFFLVSKLTVDKSRWQQGNVFSCSVMHEAL |
| | | HNHYTQKSLSLSPGK |
| | | (SEQ ID NO: 318) |
| monoFc | human | NGGPSVFLFPPKPKDTLMISRTPEVTCVVVD |
| | | VSHEDPEVKFNWYVDGVEVHNAKTKPREEQY |
| | | NSTYRVVSVLTVLHQDWLNGKEYKCKVSNKA |
| | | LPAPIEKTISKAKGQPREPQVYTLPPSREEM |
| | | TKNQVNLTCLVKGFYPSDIAVEWESNGQPEN |
| | | NYKTTPPVLDSDGSFFLNSTLTVDKSRWQQG |
| | | NVFSCSVMHEALHNHYTQKSLSLS |
| | | (SEQ ID NO: 319) |

### IL-18 Variant Leader Sequences:

The IL-18 variants described herein (e.g., the G3 substituted variants and D157 variants described above) may further comprise an N-terminal leader sequence. The leader sequence allows the expressed IL-18 variant to be secreted out of the cell, but is cleaved off during this process.

In certain embodiments, the N-terminal leader sequence comprises MYRMQLLSCIALSLALVTNS, MAAEPVEDNCINFVAMKFIDNTLYFIAEDDENLESD, MAAEPVEDNCINFVAMKFIDNTLYFIAEDDENLESD, or MAAMSEDSCVNFKEMMFIDNTLYFIPEENGDLESD.

### Exemplary IL-18 Variants, Mouse IL-18 Variant Analogs, and Comparator IL-18 Variants

Numerous exemplary IL-18 variants are described below. Table 1 describes IL-18 variants generated by rational design, as described in Example 1. Table 2 describes IL-18 variants generated by yeast display, as described in Example 1. Table 3 describes mouse IL-18 that are analogs to select human IL-18 variants of Table 1. Table 4 describes human and mouse IL-18 variant comparator sequences used in the Examples.

TABLE 1
Rationally Designed IL-18 Variants

| | Amino Acid | |
|---|---|---|
| No | Substitutions | Amino Acid Sequence |
| 1 | G3T | YFTKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 16) |
| 2 | E6R | YFGKLRSKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 17) |
| 3 | M60K | YFGKLESKLSVIRNLNDQVLFIDQGNRPLFEDMTDSDMRDNAPRTIFII |
| | | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 18) |
| 4 | T95K | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDKKSD |
| | | IIFFQRSVPGHDNKMQFESSSYEGYFLASEKERDLFKLILKKEDELGDR |
| | | SIMFTVQNED (SEQ ID NO: 19) |
| 5 | G3I | YFIKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 20) |
| 6 | N91R | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDRIKDTKSD |
| | | IIFFQRSVPGHDNKMQFESSSYEGYFLASEKERDLFKLILKKEDELGDR |
| | | SIMFTVQNED (SEQ ID NO: 21) |
| 7 | T95H | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDHKSD |
| | | IIFFQRSVPGHDNKMQFESSSYEGYFLASEKERDLFKLILKKEDELGDR |
| | | SIMFTVQNED (SEQ ID NO: 22) |
| 8 | G3V | YFVKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 23) |
| 9 | E6K | YFGKLKSKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 24) |
| 10 | E6L | YFGKLLSKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 25) |
| 11 | N91L | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDLIKDTKSD |
| | | IIFFQRSVPGHDNKMQFESSSYEGYFLASEKERDLFKLILKKEDELGDR |
| | | SIMFTVQNED (SEQ ID NO: 26) |
| 12 | E6Y | YFGKLYSKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 27) |
| 13 | M60Q | YFGKLESKLSVIRNLNDQVLFIDQGNRPLFEDMTDSDMRDNAPRTIFII |
| | | SMYKDSQPRGQAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 28) |
| 14 | E156L | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNLD (SEQ ID NO: 29) |
| 15 | M60N | YFGKLESKLSVIRNLNDQVLFIDQGNRPLFEDMTDSDMRDNAPRTIFII |
| | | SMYKDSQPRGNAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 30) |
| 16 | G3T + K53A | YFTKLESKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 31) |
| 17 | E6R + K53A | YFGKLRSKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 32) |
| 18 | M60K + K53A | YFGKLESKLSVIRNLNDQVLFIDQGNRPLFEDMTDSDMRDNAPRTIFII |
| | | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 33) |
| 19 | T95K + K53A | YFGKLESKLSVIRNLNDQVLFIDQGNRPLFEDMTDSDMRDNAPRTIFII |
| | | SMYADSQPRGMAVTISVKSEKISTLSSENKIISFKEMNPPDNIKDKKSD |
| | | IIFFQRSVPGHDNKMQFESSSYEGYFLASEKERDLFKLILKKEDELGDR |
| | | SIMFTVQNED (SEQ ID NO: 34) |
| 20 | G3I + K53A | YFIKLESKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 35) |
| 21 | N91R + K53A | YFGKLESKLSVIRNLNDQVLFIDQGNRPLFEDMTDSDMRDNAPRTIFII |
| | | SMYADSQPRGMAVTISVKSEKISTLSSENKIISFKEMNPPDRIKDTKSD |
| | | IIFFQRSVPGHDNKMQFESSSYEGYFLASEKERDLFKLILKKEDELGDR |
| | | SIMFTVQNED (SEQ ID NO: 36) |
| 22 | T95H + K53A | YFGKLESKLSVIRNLNDQVLFIDQGNRPLFEDMTDSDMRDNAPRTIFII |
| | | SMYADSQPRGMAVTISVKSEKISTLSSENKIISFKEMNPPDNIKDHKSD |
| | | IIFFQRSVPGHDNKMQFESSSYEGYFLASEKERDLFKLILKKEDELGDR |
| | | SIMFTVQNED (SEQ ID NO: 37) |
| 23 | G3V + K53A | YFVKLESKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 38) |
| 24 | E6K + K53A | YFGKLKSKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 39) |
| 25 | E6L + K53A | YFGKLLSKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 40) |
| 26 | N91L + K53A | YFGKLESKLSVIRNLNDQVLFIDQGNRPLFEDMTDSDMRDNAPRTIFII |
| | | SMYADSQPRGMAVTISVKSEKISTLSSENKIISFKEMNPPDLIKDTKSD |
| | | IIFFQRSVPGHDNKMQFESSSYEGYFLASEKERDLFKLILKKEDELGDR |
| | | SIMFTVQNED (SEQ ID NO: 41) |
| 27 | E6Y + K53A | YFGKLYSKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 42) |
| 28 | M60Q + K53A | YFGKLESKLSVIRNLNDQVLFIDQGNRPLFEDMTDSDMRDNAPRTIFII |
| | | SMYADSQPRGQAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 43) |
| 29 | E156L + K53A | YFGKLESKLSVIRNLNDQVLFIDQGNRPLFEDMTDSDMRDNAPRTIFII |
| | | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNLD (SEQ ID NO: 44) |
| 30 | M60N + K53A | YFGKLESKLSVIRNLNDQVLFIDQGNRPLFEDMTDSDMRDNAPRTIFII |
| | | SMYADSQPRGNAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 45) |
| 31 | G3T + E6K + | YFTKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 46) |
| 32 | E6R + T63A | YFGKLRSKLSVIRNLNDQVLFIDQGNRPLFEDMTDSDMRDNAPRTIFII |
| | | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 47) |
| 33 | M60K + E6K + | YFGKLKSKLSVIRNLNDQVLFIDQGNRPLFEDMTDSDMRDNAPRTIFII |
| | T63A | SMYKDSQPRGKAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 48) |
| 34 | T95K + E6K + | YFGKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDNIKDKKSD |
| | | IIFFQRSVPGHDNKMQFESSSYEGYFLASEKERDLFKLILKKEDELGDR |
| | | SIMFTVQNED (SEQ ID NO: 49) |
| 35 | G3I+ E6K + | YFIKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 50) |
| 36 | N91R + E6K + | YFGKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDRIKDTKSD |
| | | IIFFQRSVPGHDNKMQFESSSYEGYFLASEKERDLFKLILKKEDELGDR |
| | | SIMFTVQNED (SEQ ID NO: 51) |
| 37 | T95H + E6K + | YFGKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDNIKDHKSD |
| | | IIFFQRSVPGHDNKMQFESSSYEGYFLASEKERDLFKLILKKEDELGDR |
| | | SIMFTVQNED (SEQ ID NO: 52) |
| 38 | G3V+ E6K + | YFVKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 53) |
| 39 | E6K + T63A | YFGKLKSKLSVIRNLNDQVLFIDQGNRPLFEDMTDSDMRDNAPRTIFII |
| | | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 54) |
| 40 | E6L + T63A | YFGKLLSKLSVIRNLNDQVLFIDQGNRPLFEDMTDSDMRDNAPRTIFII |
| | | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 55) |
| 41 | N91L + E6K + | YFGKLKSKLSVIRNLNDQVLFIDQGNRPLFEDMTDSDMRDNAPRTIFII |
| | T63A | SMYKDSQPRGMAVAISVKSEKISTLSSENKIISFKEMNPPDLIKDTKSD |
| | | IIFFQRSVPGHDNKMQFESSSYEGYFLASEKERDLFKLILKKEDELGDR |
| | | SIMFTVQNED (SEQ ID NO: 56) |
| 42 | E6Y + T63A | YFGKLYSKLSVIRNLNDQVLFIDQGNRPLFEDMTDSDMRDNAPRTIFII |
| | | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 57) |
| 43 | M60Q + E6K + | YFGKLKSKLSVIRNLNDQVLFIDQGNRPLFEDMTDSDMRDNAPRTIFII |
| | T63A | SMYKDSQPRGQAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 58) |
| 44 | E156L + | YFGKLKSKLSVIRNLNDQVLFIDQGNRPLFEDMTDSDMRDNAPRTIFII |
| | E6K + T63A | SMYKDSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNLD (SEQ ID NO: 59) |
| 45 | M60N + E6K + | YFGKLKSKLSVIRNLNDQVLFIDQGNRPLFEDMTDSDMRDNAPRTIFII |
| | T63A | SMYKDSQPRGNAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 60) |
| 46 | G3T + E6K + | YFTKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 61) |
| 47 | E6R + T63A + | YFGKLRSKLSVIRNLNDQVLFIDQGNRPLFEDMTDSDMRDNAPRTIFII |
| | K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 62) |
| 48 | M60K + E6K + | YFGKLKSKLSVIRNLNDQVLFIDQGNRPLFEDMTDSDMRDNAPRTIFII |
| | T63A + K53A | SMYADSQPRGKAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 63) |
| 49 | T95K + E6K + | YFGKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDNIKDKKSD |
| | | IIFFQRSVPGHDNKMQFESSSYEGYFLASEKERDLFKLILKKEDELGDR |
| | | SIMFTVQNED (SEQ ID NO: 64) |
| 50 | G3I + E6K + | YFIKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 65) |
| 51 | N91R + E6K + | YFGKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDRIKDTKSD |
| | | IIFFQRSVPGHDNKMQFESSSYEGYFLASEKERDLFKLILKKEDELGDR |
| | | SIMFTVQNED (SEQ ID NO: 66) |
| 52 | T95H + E6K + | YFGKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDNIKDHKSD |
| | | IIFFQRSVPGHDNKMQFESSSYEGYFLASEKERDLFKLILKKEDELGDR |
| | | SIMFTVQNED (SEQ ID NO: 67) |
| 53 | G3V+ E6K + | YFVKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 68) |
| 54 | E6K + T63A + | YFGKLKSKLSVIRNLNDQVLFIDQGNRPLFEDMTDSDMRDNAPRTIFII |
| | K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 69) |
| 55 | E6L + T63A + | YFGKLLSKLSVIRNLNDQVLFIDQGNRPLFEDMTDSDMRDNAPRTIFII |
| | K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 70) |
| 56 | N91L + E6K + | YFGKLKSKLSVIRNLNDQVLFIDQGNRPLFEDMTDSDMRDNAPRTIFII |
| | T63A + K53A | SMYADSQPRGMAVAISVKSEKISTLSSENKIISFKEMNPPDLIKDTKSD |
| | | IIFFQRSVPGHDNKMQFESSSYEGYFLASEKERDLFKLILKKEDELGDR |
| | | SIMFTVQNED (SEQ ID NO: 71) |
| 57 | E6Y + T63A + | YFGKLYSKLSVIRNLNDQVLFIDQGNRPLFEDMTDSDMRDNAPRTIFII |
| | K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 72) |
| 58 | M60Q + E6K + | YFGKLKSKLSVIRNLNDQVLFIDQGNRPLFEDMTDSDMRDNAPRTIFII |
| | T63A + K53A | SMYADSQPRGQAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 73) |
| 59 | E156L + | YFGKLKSKLSVIRNLNDQVLFIDQGNRPLFEDMTDSDMRDNAPRTIFII |
| | E6K + T63A + | SMYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNLD (SEQ ID NO: 74) |
| 60 | M60N + E6K + | YFGKLKSKLSVIRNLNDQVLFIDQGNRPLFEDMTDSDMRDNAPRTIFII |
| | T63A + K53A | SMYADSQPRGNAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 75) |
| 61 | G3T + K53A + | YFTKLKSKLSVIRNLNDQVLFIDQGNRPLFEDMTDSDMRDNAPRTIFII |
| | E6K | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 76) |
| 62 | M60K + | YFGKLKSKLSVIRNLNDQVLFIDQGNRPLFEDMTDSDMRDNAPRTIFII |
| | K53A + E6K | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 77) |
| 63 | T95K + | YFGKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDNIKDKKSD |
| | | IIFFQRSVPGHDNKMQFESSSYEGYFLASEKERDLFKLILKKEDELGDR |
| | | SIMFTVQNED (SEQ ID NO: 78) |
| 64 | G3I + | YFIKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 79) |
| 65 | N91R + | YFGKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDRIKDTKSD |
| | | IIFFQRSVPGHDNKMQFESSSYEGYFLASEKERDLFKLILKKEDELGDR |
| | | SIMFTVQNED (SEQ ID NO: 80) |
| 66 | T95H + | YFGKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDNIKDHKSD |
| | | IIFFQRSVPGHDNKMQFESSSYEGYFLASEKERDLFKLILKKEDELGDR |
| | | SIMFTVQNED (SEQ ID NO: 81) |
| 67 | G3V + | YFVKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 82) |
| 68 | N91L + | YFGKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDLIKDTKSD |
| | | IIFFQRSVPGHDNKMQFESSSYEGYFLASEKERDLFKLILKKEDELGDR |
| | | SIMFTVQNED (SEQ ID NO: 83) |
| 69 | M60Q + | YFGKLKSKLSVIRNLNDQVLFIDQGNRPLFEDMTDSDMRDNAPRTIFII |
| | K53A + E6K | SMYADSQPRGQAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 84) |
| 70 | E156L + | YFGKLKSKLSVIRNLNDQVLFIDQGNRPLFEDMTDSDMRDNAPRTIFII |
| | K53A + E6K | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNLD (SEQ ID NO: 85) |
| 71 | M60N + | YFGKLKSKLSVIRNLNDQVLFIDQGNRPLFEDMTDSDMRDNAPRTIFII |
| | K53A + E6K | SMYADSQPRGNAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 86) |
| 72 | M51K | YFGKLESKLSVIRNLNDQVLFIDQGNRPLFEDMTDSDMRDNAPRTIFII |
| | | SKYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 87) |
| 73 | N111F | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDFKMQFESSSYEGYFLASEKERDLFKLILKKEDELGDR |
| | | SIMFTVQNED (SEQ ID NO: 88) |
| 74 | N111Q | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDQKMQFESSSYEGYFLASEKERDLFKLILKKEDELGDR |
| | | SIMFTVQNED (SEQ ID NO: 89) |
| 75 | M113V | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKVQFESSSYEGYFLASEKERDLFKLILKKEDELGDR |
| | | SIMFTVQNED (SEQ ID NO: 90) |
| 76 | M113D | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKDQFESSSYEGYFLASEKERDLFKLILKKEDELGDR |
| | | SIMFTVQNED (SEQ ID NO: 91) |
| 77 | M113K | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKKQFESSSYEGYFLASEKERDLFKLILKKEDELGDR |
| | | SIMFTVQNED (SEQ ID NO: 92) |
| 78 | M113N | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKNQFESSSYEGYFLASEKERDLFKLILKKEDELGDR |
| | | SIMFTVQNED (SEQ ID NO: 93) |
| 79 | M113Q | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKQQFESSSYEGYFLASEKERDLFKLILKKEDELGDR |
| | | SIMFTVQNED (SEQ ID NO: 94) |
| 80 | M113S | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKSQFESSSYEGYFLASEKERDLFKLILKKEDELGDR |
| | | SIMFTVQNED (SEQ ID NO: 95) |
| 81 | M113A | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKAQFESSSYEGYFLASEKERDLFKLILKKEDELGDR |
| | | SIMFTVQNED (SEQ ID NO: 96) |
| 82 | M113E | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKEQFESSSYEGYFLASEKERDLFKLILKKEDELGDR |
| | | SIMFTVQNED (SEQ ID NO: 97) |
| 83 | S105R | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRRVPGHDNKMQFESSSYEGYFLASEKERDLFKLILKKEDELGDR |
| | | SIMFTVQNED (SEQ ID NO: 98) |
| 84 | D110N | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHNNKMQFESSSYEGYFLASEKERDLFKLILKKEDELGDR |
| | | SIMFTVQNED (SEQ ID NO: 99) |
| 85 | D110T | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHTNKMQFESSSYEGYFLASEKERDLFKLILKKEDELGDR |
| | | SIMFTVQNED (SEQ ID NO: 100) |
| 86 | M51R | YFGKLESKLSVIRNLNDQVLFIDQGNRPLFEDMTDSDMRDNAPRTIFII |
| | | SRYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 101) |
| 87 | Q56E | YFGKLESKLSVIRNLNDQVLFIDQGNRPLFEDMTDSDMRDNAPRTIFII |
| | | SMYKDSEPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 102) |
| 88 | Q103A | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFARSVPGHDNKMQFESSSYEGYFLASEKERDLFKLILKKEDELGDR |
| | | SIMFTVQNED (SEQ ID NO: 103) |
| 89 | Q103V | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFVRSVPGHDNKMQFESSSYEGYFLASEKERDLFKLILKKEDELGDR |
| | | SIMFTVQNED (SEQ ID NO: 104) |
| 90 | D110Q | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHQNKMQFESSSYEGYFLASEKERDLFKLILKKEDELGDR |
| | | SIMFTVQNED (SEQ ID NO: 105) |
| 91 | D110H | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHHNKMQFESSSYEGYFLASEKERDLFKLILKKEDELGDR |
| | | SIMFTVQNED (SEQ ID NO: 106) |
| 92 | D110R | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHRNKMQFESSSYEGYFLASEKERDLFKLILKKEDELGDR |
| | | SIMFTVQNED (SEQ ID NO: 107) |
| 93 | N111D | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDDKMQFESSSYEGYFLASEKERDLFKLILKKEDELGDR |
| | | SIMFTVQNED (SEQ ID NO: 108) |
| 94 | M113I | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKIQFESSSYEGYFLASEKERDLFKLILKKEDELGDR |
| | | SIMFTVQNED (SEQ ID NO: 109) |
| 95 | N155L | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQLED (SEQ ID NO: 110) |
| 96 | N155K | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQKED (SEQ ID NO: 111) |
| 97 | N155S | YFGKLESKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQSED (SEQ ID NO: 112) |
| 98 | G59D, S105F, | YFGKLESKLSVIRNLNDQVLFIDQGNRPLFEDMTDSDMRDNAPRTIFII |
| | M113E | SMYKDSQPRDMAVTISVKSEKISTLSSENKIISFKEMNPPDNIKDTKSD |
| | | IIFFQRFVPGHDNKEQFESSSYEGYFLASEKERDLFKLILKKEDELGDR |
| | | SIMFTVQNED (SEQ ID NO: 113) |
| 99 | M60K, N155Y, | YFGKLESKLSVIRNLNDQVLFIDQGNRPLFEDMTDSDMRDNAPRTIFII |
| | D157E | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQYEE (SEQ ID NO: 114) |
| 100 | M60K, D157E | YFGKLESKLSVIRNLNDQVLFIDQGNRPLFEDMTDSDMRDNAPRTIFII |
| | | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNEE (SEQ ID NO: 115) |
| 101 | L51, E6L, | YFGKILSRLSVIRNLNDQVLFIDQGNRPLFEDMTDSDMRDNAPRTIFII |
| | K8R | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 116) |
| 102 | E6M, K8H, | YFGKLMSHLSVIRNLNDQVLFIDQGNRPLFEDMTDSDMRDNAPRTIFII |
| | M51K | SKYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 117) |
| 103 | E6M, K8H | YFGKLMSHLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 118) |
| 104 | K8H, M60K, | YFGKLESHLSVIRNLNDQVLFIDQGNRPLFEDMTDSDMRDNAPRTIFII |
| | D157R | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNER (SEQ ID NO: 119) |
| 105 | L5I, E6L, | YFGKILSKLSVIRNLNDQVLFIDQGNRPLFEDMTDSDMRDNAPRTIFII |
| | M51Y | SYYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 120) |
| 106 | L5I, E6L | YFGKILSKLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 121) |
| 107 | L5I, E6L, | YFGKILSKLSVIRNLNDQVLFIDQGNRPLFEDMTDSDMRDNAPRTIFII |
| | M51F | SFYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 122) |
| 108 | E6S, K8H, | YFGKLSSHLSVIRNLNDQVLFIDQGNRPLFEDMTDSDMRDNAPRTIFII |
| | M51K | SKYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 123) |
| 109 | E6S, K8H | YFGKLSSHLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 124) |
| 110 | E6Q, K8H, | YFGKLQSHLSVIRNLNDQVLFIDQGNRPLFEDMTDSDMRDNAPRTIFII |
| | M51K | SKYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 125) |
| 111 | E6Q, K8H | YFGKLQSHLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 126) |
| 112 | E6T, K8H, | YFGKLTSHLSVIRNLNDQVLFIDQGNRPLFEDMTDSDMRDNAPRTIFII |
| | M51K | SKYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 127) |
| 113 | E6T, K8H | YFGKLTSHLSVIRNLNDQVLFIDQGNRPLFEDMTDSDMRDNAPRTIFII |
| | | SMYKDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 128) |
| 114 | L51, E6L, | YFGKILSRLSVIRNLNDQVLFIDQGNRPLFEDMTDSDMRDNAPRTIFII |
| | K8R, T63A, | SMYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 129) |
| 115 | E6M, K8H, | YFGKLMSHLSVIRNLNDQVLFIDQGNRPLFEDMTDSDMRDNAPRTIFII |
| | M51K, T63A, | SKYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 130) |
| 116 | E6M, K8H, | YFGKLMSHLSVIRNLNDQVLFIDQGNRPLFEDMTDSDMRDNAPRTIFII |
| | T63A, K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 131) |
| 117 | L51, E6L, | YFGKILSKLSVIRNLNDQVLFIDQGNRPLFEDMTDSDMRDNAPRTIFII |
| | M51Y, T63A, | SYYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 132) |
| 118 | L51, E6L, | YFGKILSKLSVIRNLNDQVLFIDQGNRPLFEDMTDSDMRDNAPRTIFII |
| | T63A, K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 133) |
| 119 | L51, E6L, | YFGKILSKLSVIRNLNDQVLFIDQGNRPLFEDMTDSDMRDNAPRTIFII |
| | M51F, T63A, | SFYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 134) |
| 120 | E6S, K8H, | YFGKLSSHLSVIRNLNDQVLFIDQGNRPLFEDMTDSDMRDNAPRTIFII |
| | M51K, T63A, | SKYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 135) |
| 121 | E6S, K8H, | YFGKLSSHLSVIRNLNDQVLFIDQGNRPLFEDMTDSDMRDNAPRTIFII |
| | T63A, K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 136) |
| 122 | E6Q, K8H, | YFGKLQSHLSVIRNLNDQVLFIDQGNRPLFEDMTDSDMRDNAPRTIFII |
| | M51K, T63A, | SKYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 137) |
| 123 | E6Q, K8H, | YFGKLQSHLSVIRNLNDQVLFIDQGNRPLFEDMTDSDMRDNAPRTIFII |
| | T63A, K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 138) |
| 124 | E6T, K8H, | YFGKLTSHLSVIRNLNDQVLFIDQGNRPLFEDMTDSDMRDNAPRTIFII |
| | M51K, T63A, | SKYADSQPRGMAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNED (SEQ ID NO: 139) |
| 125 | E6T, K8H, | YFGKLTSHLSVIRNLNDQVLFIDQGNRPLFEDMTDSDMRDNAPRTIFII |
| | T63A, K53A | SMYADSQPRGMAVAISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 140) |
| 126 | G59D, S105F, | YFGKLASKLSVIRNLNDQVLFIDQGNRPLFEDMTDSDMRDNAPRTIFII |
| | M113E, E6A, | SMYKDSQPRDMAVAISVKSEKISTLSSENKIISFKEMNPPDNIKDTKSD |
| | T63A | IIFFQRFVPGHDNKEQFESSSYEGYFLASEKERDLFKLILKKEDELGDR |
| | | SIMFTVQNED (SEQ ID NO: 141) |
| 127 | M60K, | YFGKLASKLSVIRNLNDQVLFIDQGNRPLFEDMTDSDMRDNAPRTIFII |
| | N155Y, | SMYKDSQPRGKAVAISVKSEKISTLSSENKIISFKEMNPPDNIKDTKSD |
| | D157E, E6A, | IIFFQRSVPGHDNKMQFESSSYEGYFLASEKERDLFKLILKKEDELGDR |
| | T63A | SIMFTVQYEE (SEQ ID NO: 142) |
| 128 | M60K, | YFGKLASKLSVIRNLNDQVLFIDQGNRPLFEDMTDSDMRDNAPRTIFII |
| | D157E, E6A, | SMYKDSQPRGKAVAISVKSEKISTLSSENKIISFKEMNPPDNIKDTKSD |
| | T63A | IIFFQRSVPGHDNKMQFESSSYEGYFLASEKERDLFKLILKKEDELGDR |
| | | SIMFTVQNEE (SEQ ID NO: 143) |
| 129 | K8H, M60K, | YFGKLASHLSVIRNLNDQVLFIDQGNRPLFEDMTDSDMRDNAPRTIFII |
| | D157R, E6A, | SMYKDSQPRGKAVAISVKSEKISTLSSENKIISFKEMNPPDNIKDTKSD |
| | T63A | IIFFQRSVPGHDNKMQFESSSYEGYFLASEKERDLFKLILKKEDELGDR |
| | | SIMFTVQNER (SEQ ID NO: 144) |
| 130 | G59D, S105F, | YFGKLASKLSVIRNLNDQVLFIDQGNRPLFEDMTDSDMRDNAPRTIFII |
| | M113E, E6A, | SMYADSQPRDMAVAISVKSEKISTLSSENKIISFKEMNPPDNIKDTKSD |
| | T63A, K53A | IIFFQRFVPGHDNKEQFESSSYEGYFLASEKERDLFKLILKKEDELGDR |
| | | SIMFTVQNED (SEQ ID NO: 145) |
| 131 | M60K, N155Y, | YFGKLASKLSVIRNLNDQVLFIDQGNRPLFEDMTDSDMRDNAPRTIFII |
| | D157E, E6A, | SMYADSQPRGKAVAISVKSEKISTLSSENKIISFKEMNPPDNIKDTKSD |
| | T63A, K53A | IIFFQRSVPGHDNKMQFESSSYEGYFLASEKERDLFKLILKKEDELGDR |
| | | SIMFTVQYEE (SEQ ID NO: 146) |
| 132 | M60K, D157E, | YFGKLASKLSVIRNLNDQVLFIDQGNRPLFEDMTDSDMRDNAPRTIFII |
| | E6A, T63A, | SMYADSQPRGKAVAISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNEE (SEQ ID NO: 147) |
| 133 | K8H, M60K, | YFGKLASHLSVIRNLNDQVLFIDQGNRPLFEDMTDSDMRDNAPRTIFII |
| | D157R, E6A, | SMYADSQPRGKAVAISVKSEKISTLSSENKIISFKEMNPPDNIKDTKSD |
| | T63A, K53A | IIFFQRSVPGHDNKMQFESSSYEGYFLASEKERDLFKLILKKEDELGDR |
| | | SIMFTVQNER (SEQ ID NO: 148) |
| 134 | G59D, S105F, | YFGKLASKLSVIRNLNDQVLFIDQGNRPLFEDMTDSDMRDNAPRTIFII |
| | M113E, E6A, | SMYADSQPRDMAVTISVKSEKISTLSSENKIISFKEMNPPDNIKDTKSD |
| | K53A | IIFFQRFVPGHDNKEQFESSSYEGYFLASEKERDLFKLILKKEDELGDR |
| | | SIMFTVQNED (SEQ ID NO: 149) |
| 135 | M60K, N155Y, | YFGKLASKLSVIRNLNDQVLFIDQGNRPLFEDMTDSDMRDNAPRTIFII |
| | D157E, E6A, | SMYADSQPRGKAVTISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQYEE (SEQ ID NO: 150) |
| 136 | M60K, D157E, | YFGKLASKLSVIRNLNDQVLFIDQGNRPLFEDMTDSDMRDNAPRTIFII |
| | E6A, K53A | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNEE (SEQ ID NO: 151) |
| 137 | K8H, M60K, | YFGKLASHLSVIRNLNDQVLFIDQGNRPLFEDMTDSDMRDNAPRTIFII |
| | D157R, E6A, | SMYADSQPRGKAVTISVKSEKISTLSSENKIISFKEMNPPDNIKDTKSD |
| | K53A | IIFFQRSVPGHDNKMQFESSSYEGYFLASEKERDLFKLILKKEDELGDR |
| | | SIMFTVQNER (SEQ ID NO: 152) |
| 138 | G3T, M60K | YFTKLESKLSVIRNLNDQVLFIDQGNRPLFEDMTDSDMRDNAPRTIFII |
| | | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 11) |
| 139 | G3V, M60K | YFVKLESKLSVIRNLNDQVLFIDQGNRPLFEDMTDSDMRDNAPRTIFII |
| | | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 10) |
| 140 | G3T, M60K, | YFTKLESKLSVIRNLNDQVLFIDQGNRPLFEDMTDSDMRDNAPRTIFII |
| | D157E | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNEE (SEQ ID NO: 15) |
| 141 | G3V, M60K, | YFVKLESKLSVIRNLNDQVLFIDQGNRPLFEDMTDSDMRDNAPRTIFII |
| | D157E | SMYKDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNEE (SEQ ID NO: 14) |
| 142 | G3R, N91M, | YFRKLESKLSVIRNLNDQVLFIDQGNRPLFEDMTDSDMRDNAPRTIFII |
| | K53A | SMYADSQPRGMAVTISVKSEKISTLSSENKIISFKEMNPPDMIKDTKSD |
| | | IIFFQRSVPGHDNKMQFESSSYEGYFLASEKERDLFKLILKKEDELGDR |
| | | SIMFTVQNED (SEQ ID NO: 153) |
| 143 | G3E, K53S | YFEKLESKLSVIRNLNDQVLFIDQGNRPLFEDMTDSDMRDNAPRTIFII |
| | | SMYSDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 154) |
| 144 | G3S, K53S | YFSKLESKLSVIRNLNDQVLFIDQGNRPLFEDMTDSDMRDNAPRTIFII |
| | | SMYSDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 155) |
| 145 | G3A, K53S | YFAKLESKLSVIRNLNDQVLFIDQGNRPLFEDMTDSDMRDNAPRTIFII |
| | | SMYSDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 156) |
| 146 | G3R, K53S | YFRKLESKLSVIRNLNDQVLFIDQGNRPLFEDMTDSDMRDNAPRTIFII |
| | | SMYSDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 157) |
| 147 | G3F, K53S | YFFKLESKLSVIRNLNDQVLFIDQGNRPLFEDMTDSDMRDNAPRTIFII |
| | | SMYSDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 158) |
| 148 | G3T, K53S | YFTKLESKLSVIRNLNDQVLFIDQGNRPLFEDMTDSDMRDNAPRTIFII |
| | | SMYSDSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 159) |
| 149 | G3F, N91E, | YFFKLESKLSVIRNLNDQVLFIDQGNRPLFEDMTDSDMRDNAPRTIFII |
| | K53S | SMYSDSQPRGMAVTISVKSEKISTLSSENKIISFKEMNPPDEIKDTKSD |
| | | IIFFQRSVPGHDNKMQFESSSYEGYFLASEKERDLFKLILKKEDELGDR |
| | | SIMFTVQNED (SEQ ID NO: 160) |
| 150 | G3R, N91A, | YFRKLESKLSVIRNLNDQVLFIDQGNRPLFEDMTDSDMRDNAPRTIFII |
| | K53S | SMYSDSQPRGMAVTISVKSEKISTLSSENKIISFKEMNPPDAIKDTKSD |
| | | IIFFQRSVPGHDNKMQFESSSYEGYFLASEKERDLFKLILKKEDELGDR |
| | | SIMFTVQNED (SEQ ID NO: 161) |
| 151 | G3R, N91S, | YFRKLESKLSVIRNLNDQVLFIDQGNRPLFEDMTDSDMRDNAPRTIFII |
| | K53A | SMYADSQPRGMAVTISVKSEKISTLSSENKIISFKEMNPPDSIKDTKSD |
| | | IIFFQRSVPGHDNKMQFESSSYEGYFLASEKERDLFKLILKKEDELGDR |
| | | SIMFTVQNED (SEQ ID NO: 162) |
| 152 | G3H, N91V, | YFHKLESKLSVIRNLNDQVLFIDQGNRPLFEDMTDSDMRDNAPRTIFII |
| | K53S | SMYSDSQPRGMAVTISVKSEKISTLSSENKIISFKEMNPPDVIKDTKSD |
| | | IIFFQRSVPGHDNKMQFESSSYEGYFLASEKERDLFKLILKKEDELGDR |
| | | SIMFTVQNED (SEQ ID NO: 163) |
| 153 | G3R, N91W, | YFRKLESKLSVIRNLNDQVLFIDQGNRPLFEDMTDSDMRDNAPRTIFII |
| | K53A | SMYADSQPRGMAVTISVKSEKISTLSSENKIISFKEMNPPDWIKDTKSD |
| | | IIFFQRSVPGHDNKMQFESSSYEGYFLASEKERDLFKLILKKEDELGDR |
| | | SIMFTVQNED (SEQ ID NO: 164) |
| 154 | G3R, N91M, | YFRKLESKLSVIRNLNDQVLFIDQGNRPLFEDMTDSDMRDNAPRTIFII |
| | K53S | SMYSDSQPRGMAVTISVKSEKISTLSSENKIISFKEMNPPDMIKDTKSD |
| | | IIFFQRSVPGHDNKMQFESSSYEGYFLASEKERDLFKLILKKEDELGDR |
| | | SIMFTVQNED (SEQ ID NO: 165) |
| 155 | G3K, N91I, | YFKKLESKLSVIRNLNDQVLFIDQGNRPLFEDMTDSDMRDNAPRTIFII |
| | K53S | SMYSDSQPRGMAVTISVKSEKISTLSSENKIISFKEMNPPDIIKDTKSD |
| | | IIFFQRSVPGHDNKMQFESSSYEGYFLASEKERDLFKLILKKEDELGDR |
| | | SIMFTVQNED (SEQ ID NO: 166) |
| 156 | G3A, N91L, | YFAKLESKLSVIRNLNDQVLFIDQGNRPLFEDMTDSDMRDNAPRTIFII |
| | K53S | SMYSDSQPRGMAVTISVKSEKISTLSSENKIISFKEMNPPDLIKDTKSD |
| | | IIFFQRSVPGHDNKMQFESSSYEGYFLASEKERDLFKLILKKEDELGDR |
| | | SIMFTVQNED (SEQ ID NO: 167) |
| 157 | G3V, E6K, | YFVKLKSKLSVIRNLNDQVLFIDQGNRPLFEDMTDSDMRDNAPRTIFII |
| | K53S, M60K | SMYSDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 5) |
| 158 | G3V, E6K, | YFVKLKSKLSVIRNLNDQVLFIDQGNRPLFEDMTDSDMRDNAPRTIFII |
| | K53A, M60K | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 2) |
| 159 | G3T, E6K, | YFTKLKSKLSVIRNLNDQVLFIDQGNRPLFEDMTDSDMRDNAPRTIFII |
| | K53S, M60K | SMYSDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 4) |
| 160 | G3T, E6K, | YFTKLKSKLSVIRNLNDQVLFIDQGNRPLFEDMTDSDMRDNAPRTIFII |
| | K53A, M60K | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 3) |
| 161 | G3V, K53S, | YFVKLESKLSVIRNLNDQVLFIDQGNRPLFEDMTDSDMRDNAPRTIFII |
| | M60K | SMYSDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 9) |
| 162 | G3V, K53A, | YFVKLESKLSVIRNLNDQVLFIDQGNRPLFEDMTDSDMRDNAPRTIFII |
| | M60K | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 8) |
| 163 | G3T, K53S, | YFTKLESKLSVIRNLNDQVLFIDQGNRPLFEDMTDSDMRDNAPRTIFII |
| | M60K | SMYSDSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 7) |
| 164 | G3T, K53A, | YFTKLESKLSVIRNLNDQVLFIDQGNRPLFEDMTDSDMRDNAPRTIFII |
| | M60K | SMYADSQPRGKAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 6) |
| 165 | G3V, E6K, | YFVKLKSKLSVIRNLNDQVLFIDQGNRPLFEDMTDSDMRDNAPRTIFII |
| | K53A | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 12) |
| 166 | G3T, E6K, | YFTKLKSKLSVIRNLNDQVLFIDQGNRPLFEDMTDSDMRDNAPRTIFII |
| | K53A | SMYADSQPRGMAVTISVKSEKISTLSSENKIISFKEMNPPDNIKDTKSD |
| | | IIFFQRSVPGHDNKMQFESSSYEGYFLASEKERDLFKLILKKEDELGDR |
| | | SIMFTVQNED (SEQ ID NO: 13) |

TABLE 2
Yeast Display IL-18 Variants. “X” corresponds to an amino acid deletion.

| No | Amino Acid Substitutions | Sequence |
|---|---|---|
| 1 | E6D, S36F, K53T, S55M, | YFGKLDSKLSVIRNLNDQVLFIDQGNRPLFEDMTDFDM |
| | R58K | RDNAPRTIFIISMYTDMQPKGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 168) |
| 2 | L15I, K53H, S55M | YFGKLESKLSVIRNINDQVLFIDQGNRPLFEDMTDSDM |
| | | RDNAPRTIFIISMYHDMQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 169) |
| 3 | T34E, S36P, D40A, N41G, | YFGKLESKLSVIRNLNDQVLFIDQGNRPLFEDMEDPDM |
| | K53T, S55R | RAGAPRTIFIISMYTDRQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 170) |
| 4 | T34E, N41D, K53N, S55R | YFGKLESKLSVIRNLNDQVLFIDQGNRPLFEDMEDSDM |
| | | RDDAPRTIFIISMYNDRQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 171) |
| 5 | T34I, S36V, D40I, N41M, | YFGKLESKLSVIRNLNDQVLFIDQGNRPLFEDMIDVDM |
| | K53G, S55Y | RIMAPRTIFIISMYGDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 172) |
| 6 | T34N, S36N, D40E, N41G, | YFGKLESKLSVIRNLNDQVLFIDQGNRPLFEDMNDNDM |
| | K53S, S55Q, R104N, | REGAPRTIFIISMYSDQQPRGMAVTISVKSEKISTLSSEN |
| | H109Y | KIISFKEMNPPDNIKDTKSDIIFFQNSVPGYDNKMQFESS |
| | | SYEGYFLASEKERDLFKLILKKEDELGDRSIMFTVQNED |
| | | (SEQ ID NO: 173) |
| 7 | T34N, S36Y, D40H, N41T, | YFGKLESKLSVIRNLNDQVLFIDQGNRPLFEDMNDYDM |
| | K53G, S55Y | RHTAPRTIFIISMYGDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 174) |
| 8 | T34N, S36Y, D37E, D40Q, | YFGKLESKLSVIRNLNDQVLFIDQGNRPLFEDMNDYEM |
| | N41Q, K53D, S55D | RQQAPRTIFIISMYDDDQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 175) |
| 9 | T34S, S36L, D37E, D40Y, | YFGKLESKLSVIRNLNDQVLFIDQGNRPLFEDMSDLEM |
| | N41M, K53T, S55K, | RYMAPRTIFIISMYTDKQPRGMAVTISVKSEKISTLSSEN |
| | P107K, H109Q | KIISFKEMNPPDNIKDTKSDIIFFQRSVKGQDNKMQFESS |
| | | SYEGYFLASEKERDLFKLILKKEDELGDRSIMFTVQNED |
| | | (SEQ ID NO: 176) |
| 10 | T34S, S36Y, D37N, D40E, | YFGKLESKLSVIRNLNDQVLFIDQGNRPLFEDMSDYNM |
| | N41Q, K53Y, S55I | REQAPRTIFIISMYYDIQPRGMAVTISVKSEKISTLSSENK |
| | | IISFKEMNPPDNIKDTKSDIIFFQRSVPGHDNKMQFESSS |
| | | YEGYFLASEKERDLFKLILKKEDELGDRSIMFTVQNED |
| | | (SEQ ID NO: 177) |
| 11 | S36E, D40A, N41M, K53T, | YFGKLESKLSVIRNLNDQVLFIDQGNRPLFEDMTDEDM |
| | S55Y, P107S | RAMAPRTIFIISMYTDYQPRGMAVTISVKSEKISTLSSEN |
| | | KIISFKEMNPPDNIKDTKSDIIFFQRSVSGHDNKMQFESS |
| | | SYEGYFLASEKERDLFKLILKKEDELGDRSIMFTVQNED |
| | | (SEQ ID NO: 178) |
| 12 | S36E, D37E, D40V, N41S, | YFGKLESKLSVIRNLNDQVLFIDQGNRPLFEDMTDEEM |
| | K53Y, S55V, H109Y | RVSAPRTIFIISMYYDVQPRGMAVTISVKSEKISTLSSEN |
| | | KIISFKEMNPPDNIKDTKSDIIFFQRSVPGYDNKMQFESS |
| | | SYEGYFLASEKERDLFKLILKKEDELGDRSIMFTVQNED |
| | | (SEQ ID NO: 179) |
| 13 | S36F, N41E, K53W | YFGKLESKLSVIRNLNDQVLFIDQGNRPLFEDMTDFDM |
| | | RDEAPRTIFIISMYWDSQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 180) |
| 14 | S36Q, D40T, K53P, S55L | YFGKLESKLSVIRNLNDQVLFIDQGNRPLFEDMTDQDM |
| | | RTNAPRTIFIISMYPDLQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 181) |
| 15 | K53X, S55A | YFGKLESKLSVIRNLNDQVLFIDQGNRPLFEDMTDSDM |
| | | RDNAPRTIFIISMYXDAQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 182) |
| 16 | K53D, S55E | YFGKLESKLSVIRNLNDQVLFIDQGNRPLFEDMTDSDM |
| | | RDNAPRTIFIISMYDDEQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 183) |
| 17 | K53D, S55L | YFGKLESKLSVIRNLNDQVLFIDQGNRPLFEDMTDSDM |
| | | RDNAPRTIFIISMYDDLQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 184) |
| 18 | K53D, S55N | YFGKLESKLSVIRNLNDQVLFIDQGNRPLFEDMTDSDM |
| | | RDNAPRTIFIISMYDDNQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 185) |
| 19 | K53D, S55N, H109I | YFGKLESKLSVIRNLNDQVLFIDQGNRPLFEDMTDSDM |
| | | RDNAPRTIFIISMYDDNQPRGMAVTISVKSEKISTLSSEN |
| | | KIISFKEMNPPDNIKDTKSDIIFFQRSVPGIDNKMQFESSS |
| | | YEGYFLASEKERDLFKLILKKEDELGDRSIMFTVQNED |
| | | (SEQ ID NO: 186) |
| 20 | K53D, S55T, H109I | YFGKLESKLSVIRNLNDQVLFIDQGNRPLFEDMTDSDM |
| | | RDNAPRTIFIISMYDDTQPRGMAVTISVKSEKISTLSSEN |
| | | KIISFKEMNPPDNIKDTKSDIIFFQRSVPGIDNKMQFESSS |
| | | YEGYFLASEKERDLFKLILKKEDELGDRSIMFTVQNED |
| | | (SEQ ID NO: 187) |
| 21 | K53G, S55I, R104K, | YFGKLESKLSVIRNLNDQVLFIDQGNRPLFEDMTDSDM |
| | P107Y | RDNAPRTIFIISMYGDIQPRGMAVTISVKSEKISTLSSEN |
| | | KIISFKEMNPPDNIKDTKSDIIFFQKSVYGHDNKMQFESS |
| | | SYEGYFLASEKERDLFKLILKKEDELGDRSIMFTVQNED |
| | | (SEQ ID NO: 188) |
| 22 | K53G, S55I | YFGKLESKLSVIRNLNDQVLFIDQGNRPLFEDMTDSDM |
| | | RDNAPRTIFIISMYGDIQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 189) |
| 23 | K53G, S55P, R104K, | YFGKLESKLSVIRNLNDQVLFIDQGNRPLFEDMTDSDM |
| | H109Y | RDNAPRTIFIISMYGDPQPRGMAVTISVKSEKISTLSSEN |
| | | KIISFKEMNPPDNIKDTKSDIIFFQKSVPGYDNKMQFESS |
| | | SYEGYFLASEKERDLFKLILKKEDELGDRSIMFTVQNED |
| | | (SEQ ID NO: 190) |
| 24 | K53G, S55V | YFGKLESKLSVIRNLNDQVLFIDQGNRPLFEDMTDSDM |
| | | RDNAPRTIFIISMYGDVQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 191) |
| 25 | K53H, S55G | YFGKLESKLSVIRNLNDQVLFIDQGNRPLFEDMTDSDM |
| | | RDNAPRTIFIISMYHDGQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 192) |
| 26 | K53H, S55H, N87D, | YFGKLESKLSVIRNLNDQVLFIDQGNRPLFEDMTDSDM |
| | H109I | RDNAPRTIFIISMYHDHQPRGMAVTISVKSEKISTLSSEN |
| | | KIISFKEMDPPDNIKDTKSDIIFFQRSVPGIDNKMQFESSS |
| | | YEGYFLASEKERDLFKLILKKEDELGDRSIMFTVQNED |
| | | (SEQ ID NO: 193) |
| 27 | K53H, S55H | YFGKLESKLSVIRNLNDQVLFIDQGNRPLFEDMTDSDM |
| | | RDNAPRTIFIISMYHDHQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 194) |
| 28 | K53H, S55I | YFGKLESKLSVIRNLNDQVLFIDQGNRPLFEDMTDSDM |
| | | RDNAPRTIFIISMYHDIQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 195) |
| 29 | K53H, S55M, Q56H | YFGKLESKLSVIRNLNDQVLFIDQGNRPLFEDMTDSDM |
| | | RDNAPRTIFIISMYHDMHPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 196) |
| 30 | K53H, S55M, T63S | YFGKLESKLSVIRNLNDQVLFIDQGNRPLFEDMTDSDM |
| | | RDNAPRTIFIISMYHDMQPRGMAVSISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 197) |
| 31 | K53H, S55M | YFGKLESKLSVIRNLNDQVLFIDQGNRPLFEDMTDSDM |
| | | RDNAPRTIFIISMYHDMQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 198) |
| 32 | K53H, S55Q | YFGKLESKLSVIRNLNDQVLFIDQGNRPLFEDMTDSDM |
| | | RDNAPRTIFIISMYHDQQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 199) |
| 33 | K53H, S55R | YFGKLESKLSVIRNLNDQVLFIDQGNRPLFEDMTDSDM |
| | | RDNAPRTIFIISMYHDRQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 200) |
| 34 | K53H, S55V | YFGKLESKLSVIRNLNDQVLFIDQGNRPLFEDMTDSDM |
| | | RDNAPRTIFIISMYHDVQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 201) |
| 35 | K53H, S55Y, Q56H | YFGKLESKLSVIRNLNDQVLFIDQGNRPLFEDMTDSDM |
| | | RDNAPRTIFIISMYHDYHPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 202) |
| 36 | K53H, S55Y | YFGKLESKLSVIRNLNDQVLFIDQGNRPLFEDMTDSDM |
| | | RDNAPRTIFIISMYHDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 203) |
| 37 | K53L, S55E | YFGKLESKLSVIRNLNDQVLFIDQGNRPLFEDMTDSDM |
| | | RDNAPRTIFIISMYLDEQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 204) |
| 38 | K53L, S55G | YFGKLESKLSVIRNLNDQVLFIDQGNRPLFEDMTDSDM |
| | | RDNAPRTIFIISMYLDGQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 205) |
| 39 | K53N, S55H, H109F | YFGKLESKLSVIRNLNDQVLFIDQGNRPLFEDMTDSDM |
| | | RDNAPRTIFIISMYNDHQPRGMAVTISVKSEKISTLSSEN |
| | | KIISFKEMNPPDNIKDTKSDIIFFQRSVPGFDNKMQFESS |
| | | SYEGYFLASEKERDLFKLILKKEDELGDRSIMFTVQNED |
| | | (SEQ ID NO: 206) |
| 40 | K53N, S55I | YFGKLESKLSVIRNLNDQVLFIDQGNRPLFEDMTDSDM |
| | | RDNAPRTIFIISMYNDIQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 207) |
| 41 | K53N, S55Y | YFGKLESKLSVIRNLNDQVLFIDQGNRPLFEDMTDSDM |
| | | RDNAPRTIFIISMYNDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 208) |
| 42 | K53Q, D54N, S55M, Q56H | YFGKLESKLSVIRNLNDQVLFIDQGNRPLFEDMTDSDM |
| | | RDNAPRTIFIISMYQNMHPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 209) |
| 43 | K53S, S55F, T63S, | YFGKLESKLSVIRNLNDQVLFIDQGNRPLFEDMTDSDM |
| | R104K, H109Y | RDNAPRTIFIISMYSDFQPRGMAVSISVKSEKISTLSSEN |
| | | KIISFKEMNPPDNIKDTKSDIIFFQKSVPGYDNKMQFESS |
| | | SYEGYFLASEKERDLFKLILKKEDELGDRSIMFTVQNED |
| | | (SEQ ID NO: 210) |
| 44 | K53S, S55F, R104K, | YFGKLESKLSVIRNLNDQVLFIDQGNRPLFEDMTDSDM |
| | H109Y | RDNAPRTIFIISMYSDFQPRGMAVTISVKSEKISTLSSEN |
| | | KIISFKEMNPPDNIKDTKSDIIFFQKSVPGYDNKMQFESS |
| | | SYEGYFLASEKERDLFKLILKKEDELGDRSIMFTVQNED |
| | | (SEQ ID NO: 211) |
| 45 | K53S, S55G | YFGKLESKLSVIRNLNDQVLFIDQGNRPLFEDMTDSDM |
| | | RDNAPRTIFIISMYSDGQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 212) |
| 46 | K53S, S55K | YFGKLESKLSVIRNLNDQVLFIDQGNRPLFEDMTDSDM |
| | | RDNAPRTIFIISMYSDKQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 213) |
| 47 | K53S, S55Y, P107M | YFGKLESKLSVIRNLNDQVLFIDQGNRPLFEDMTDSDM |
| | | RDNAPRTIFIISMYSDYQPRGMAVTISVKSEKISTLSSEN |
| | | KIISFKEMNPPDNIKDTKSDIIFFQRSVMGHDNKMQFES |
| | | SSYEGYFLASEKERDLFKLILKKEDELGDRSIMFTVQNE |
| | | D (SEQ ID NO: 214) |
| 48 | K53S, S55Y | YFGKLESKLSVIRNLNDQVLFIDQGNRPLFEDMTDSDM |
| | | RDNAPRTIFIISMYSDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 215) |
| 49 | K53T, S55A | YFGKLESKLSVIRNLNDQVLFIDQGNRPLFEDMTDSDM |
| | | RDNAPRTIFIISMYTDAQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 216) |
| 50 | K53T, S55F | YFGKLESKLSVIRNLNDQVLFIDQGNRPLFEDMTDSDM |
| | | RDNAPRTIFIISMYTDFQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 217) |
| 51 | K53T, S55G, P107A, | YFGKLESKLSVIRNLNDQVLFIDQGNRPLFEDMTDSDM |
| | H109Q | RDNAPRTIFIISMYTDGQPRGMAVTISVKSEKISTLSSEN |
| | | KIISFKEMNPPDNIKDTKSDIIFFQRSVAGQDNKMQFESS |
| | | SYEGYFLASEKERDLFKLILKKEDELGDRSIMFTVQNED |
| | | (SEQ ID NO: 218) |
| 52 | K53T, S55M, R58K | YFGKLESKLSVIRNLNDQVLFIDQGNRPLFEDMTDSDM |
| | | RDNAPRTIFIISMYTDMQPKGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 219) |
| 53 | K53T, S55P | YFGKLESKLSVIRNLNDQVLFIDQGNRPLFEDMTDSDM |
| | | RDNAPRTIFIISMYTDPQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 220) |
| 54 | K53W, S55A | YFGKLESKLSVIRNLNDQVLFIDQGNRPLFEDMTDSDM |
| | | RDNAPRTIFIISMYWDAQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 221) |
| 55 | K53Y, S55G, Q56H | YFGKLESKLSVIRNLNDQVLFIDQGNRPLFEDMTDSDM |
| | | RDNAPRTIFIISMYYDGHPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 222) |
| 56 | K53Y, S55G | YFGKLESKLSVIRNLNDQVLFIDQGNRPLFEDMTDSDM |
| | | RDNAPRTIFIISMYYDGQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 223) |
| 57 | K53Y, S55V | YFGKLESKLSVIRNLNDQVLFIDQGNRPLFEDMTDSDM |
| | | RDNAPRTIFIISMYYDVQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 224) |
| 58 | K53Y, S55Y, Q56H | YFGKLESKLSVIRNLNDQVLFIDQGNRPLFEDMTDSDM |
| | | RDNAPRTIFIISMYYDYHPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 225) |
| 59 | K53Y, S55Y | YFGKLESKLSVIRNLNDQVLFIDQGNRPLFEDMTDSDM |
| | | RDNAPRTIFIISMYYDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 226) |
| 60 | S36W, D37E, D40F, N41I, | YFGKLESKLSVIRNLNDQVLFIDQGNRPLFEDMTDWEM |
| | K53D, S55N | RFIAPRTIFIISMYDDNQPRGMAVTISVKSEKISTLSSENK |
| | | IISFKEMNPPDNIKDTKSDIIFFQRSVPGHDNKMQFESSS |
| | | YEGYFLASEKERDLFKLILKKEDELGDRSIMFTVQNED |
| | | (SEQ ID NO: 227) |
| 61 | S36Y, D37E, D40X, N41M, | YFGKLESKLSVIRNLNDQVLFIDQGNRPLFEDMTDYEM |
| | K53S, S55L | RXMAPRTIFIISMYSDLQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 228) |
| 62 | D23G, K53Y, S55Y | YFGKLESKLSVIRNLNDQVLFIGQGNRPLFEDMTDSDM |
| | | RDNAPRTIFIISMYYDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 229) |
| 63 | R13S, D23H, R27T, F30L, | YFGKLESKLSVISNLNDQVLFIHQGNTPLLEDMDDYEX |
| | T34D, S36Y, D37E, M38X, | RRMAPRTIFIISMYSDLQPRGMAVTISVKSEKISTLSSEN |
| | D40R, N41M, K53S, S55L | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 230) |
| 64 | L9V, K53H, S55M | YFGKLESKVSVIRNLNDQVLFIDQGNRPLFEDMTDSDM |
| | | RDNAPRTIFIISMYHDMQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 231) |
| 65 | S7Y, K53H, S55M | YFGKLEYKLSVIRNLNDQVLFIDQGNRPLFEDMTDSDM |
| | | RDNAPRTIFIISMYHDMQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 232) |
| 66 | L5W, 122M, K53N, S55Y, | YFGKWESKLSVIRNLNDQVLFMDQGNRPLFEDMTDSD |
| | Q56H | MRDNAPRTIFIISMYNDYHPRGMAVTISVKSEKISTLSSE |
| | | NKIISFKEMNPPDNIKDTKSDIIFFQRSVPGHDNKMQFES |
| | | SSYEGYFLASEKERDLFKLILKKEDELGDRSIMFTVQNE |
| | | D (SEQ ID NO: 233) |
| 67 | L5W, S7Y, K53H, S55M | YFGKWEYKLSVIRNLNDQVLFIDQGNRPLFEDMTDSD |
| | | MRDNAPRTIFIISMYHDMQPRGMAVTISVKSEKISTLSS |
| | | ENKIISFKEMNPPDNIKDTKSDIIFFQRSVPGHDNKMQFE |
| | | SSSYEGYFLASEKERDLFKLILKKEDELGDRSIMFTVQN |
| | | ED (SEQ ID NO: 234) |
| 68 | K53N, S55A, R104N, | YFGKLESKLSVIRNLNDQVLFIDQGNRPLFEDMTDSDM |
| | P107A, H109Y | RDNAPRTIFIISMYNDAQPRGMAVTISVKSEKISTLSSEN |
| | | KIISFKEMNPPDNIKDTKSDIIFFQNSVAGYDNKMQFESS |
| | | SYEGYFLASEKERDLFKLILKKEDELGDRSIMFTVQNED |
| | | (SEQ ID NO: 235) |
| 69 | T34I, S36T, D40A, N41M, | YFGKLESKLSVIRNLNDQVLFIDQGNRPLFEDMIDTDM |
| | K53T, S55Y | RAMAPRTIFIISMYTDYQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 236) |
| 70 | T34L, S36V, D40A, N41S, | YFGKLESKLSVIRNLNDQVLFIDQGNRPLFEDMLDVDM |
| | K53G, S55W | RASAPRTIFIISMYGDWQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 237) |
| 71 | T34M, S36T, D37E, D40T, | YFGKLESKLSVIRNLNDQVLFIDQGNRPLFEDMMDTEM |
| | N41M, K53T, S55W | RTMAPRTIFIISMYTDWQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 238) |
| 72 | S36E, D37E, D40K, N41T, | YFGKLESKLSVIRNLNDQVLFIDQGNRPLFEDMTDEEM |
| | K53T, S55Q, R104H | RKTAPRTIFIISMYTDQQPRGMAVTISVKSEKISTLSSEN |
| | | KIISFKEMNPPDNIKDTKSDIIFFQHSVPGHDNKMQFESS |
| | | SYEGYFLASEKERDLFKLILKKEDELGDRSIMFTVQNED |
| | | (SEQ ID NO: 239) |
| 73 | S36E, D37E, D40R, N41G, | YFGKLESKLSVIRNLNDQVLFIDQGNRPLFEDMTDEEM |
| | K53Y, S55F | RRGAPRTIFIISMYYDFQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 240) |
| 74 | K53D, S55Q, P107M | YFGKLESKLSVIRNLNDQVLFIDQGNRPLFEDMTDSDM |
| | | RDNAPRTIFIISMYDDQQPRGMAVTISVKSEKISTLSSEN |
| | | KIISFKEMNPPDNIKDTKSDIIFFQRSVMGHDNKMQFES |
| | | SSYEGYFLASEKERDLFKLILKKEDELGDRSIMFTVQNE |
| | | D (SEQ ID NO: 241) |
| 75 | K53G, S55V, R104Y, | YFGKLESKLSVIRNLNDQVLFIDQGNRPLFEDMTDSDM |
| | H109N | RDNAPRTIFIISMYGDVQPRGMAVTISVKSEKISTLSSEN |
| | | KIISFKEMNPPDNIKDTKSDIIFFQYSVPGNDNKMQFESS |
| | | SYEGYFLASEKERDLFKLILKKEDELGDRSIMFTVQNED |
| | | (SEQ ID NO: 242) |
| 76 | K53N, S55M, R104S, | YFGKLESKLSVIRNLNDQVLFIDQGNRPLFEDMTDSDM |
| | P107A | RDNAPRTIFIISMYNDMQPRGMAVTISVKSEKISTLSSEN |
| | | KIISFKEMNPPDNIKDTKSDIIFFQSSVAGHDNKMQFESS |
| | | SYEGYFLASEKERDLFKLILKKEDELGDRSIMFTVQNED |
| | | (SEQ ID NO: 243) |
| 77 | K8R, L9G, S10C, K53G, | YFGKLESRGCVIRNLNDQVLFIDQGNRPLFEDMTDSDM |
| | S55A, R104G | RDNAPRTIFIISMYGDAQPRGMAVTISVKSEKISTLSSEN |
| | | KIISFKEMNPPDNIKDTKSDIIFFQGSVPGHDNKMQFESS |
| | | SYEGYFLASEKERDLFKLILKKEDELGDRSIMFTVQNED |
| | | (SEQ ID NO: 244) |
| 78 | K53V, S55Q | YFGKLESKLSVIRNLNDQVLFIDQGNRPLFEDMTDSDM |
| | | RDNAPRTIFIISMYVDQQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 245) |
| 79 | K53D, S55Q, H109Y | YFGKLESKLSVIRNLNDQVLFIDQGNRPLFEDMTDSDM |
| | | RDNAPRTIFIISMYDDQQPRGMAVTISVKSEKISTLSSEN |
| | | KIISFKEMNPPDNIKDTKSDIIFFQRSVPGYDNKMQFESS |
| | | SYEGYFLASEKERDLFKLILKKEDELGDRSIMFTVQNED |
| | | (SEQ ID NO: 246) |
| 80 | G3D, E6G, K53G, D54A, | YFDKLGSKLSVIRNLNDQVLFIDQGNRPLFEDMTDSDM |
| | T63S, N91Y, R104Q | RDNAPRTIFIISMYGASQPRGMAVSISVKSEKISTLSSEN |
| | | KIISFKEMNPPDYIKDTKSDIIFFQQSVPGHDNKMQFESS |
| | | SYEGYFLASEKERDLFKLILKKEDELGDRSIMFTVQNED |
| | | (SEQ ID NO: 247) |
| 81 | G3D, E6Q, R27S, K53R, | YFDKLQSKLSVIRNLNDQVLFIDQGNSPLFEDMTDSDM |
| | D54A, S55R, T63S, N91Q | RDNAPRTIFIISMYRARQPRGMAVSISVKSEKISTLSSEN |
| | | KIISFKEMNPPDQIKDTKSDIIFFQRSVPGHDNKMQFESS |
| | | SYEGYFLASEKERDLFKLILKKEDELGDRSIMFTVQNED |
| | | (SEQ ID NO: 248) |
| 82 | G3E, E6A, K53Q, D54W, | YFEKLASKLSVIRNLNDQVLFIDQGNRPLFEDMTDSDM |
| | S55V, T63M, N91M, | RDNAPRTIFIISMYQWVQPRGMAVMISVKSEKISTLSSE |
| | R104N | NKIISFKEMNPPDMIKDTKSDIIFFQNSVPGHDNKMQFE |
| | | SSSYEGYFLASEKERDLFKLILKKEDELGDRSIMFTVQN |
| | | ED (SEQ ID NO: 249) |
| 83 | G3E, E6G, N91M, R104A | YFEKLGSKLSVIRNLNDQVLFIDQGNRPLFEDMTDSDM |
| | | RDNAPRTIFIISMYKDSQPRGMAVTISVKSEKISTLSSEN |
| | | KIISFKEMNPPDMIKDTKSDIIFFQASVPGHDNKMQFESS |
| | | SYEGYFLASEKERDLFKLILKKEDELGDRSIMFTVQNED |
| | | (SEQ ID NO: 250) |
| 84 | G3E, E6H, R27S, D54A, | YFEKLHSKLSVIRNLNDQVLFIDQGNSPLFEDMTDSDM |
| | S55T, T63S, N91F, R104S | RDNAPRTIFIISMYKATQPRGMAVSISVKSEKISTLSSEN |
| | | KIISFKEMNPPDFIKDTKSDIIFFQSSVPGHDNKMQFESSS |
| | | YEGYFLASEKERDLFKLILKKEDELGDRSIMFTVQNED |
| | | (SEQ ID NO: 251) |
| 85 | E6D, D54T, S55W, T63S, | YFGKLDSKLSVIRNLNDQVLFIDQGNRPLFEDMTDSDM |
| | N91S, R104T | RDNAPRTIFIISMYKTWQPRGMAVSISVKSEKISTLSSEN |
| | | KIISFKEMNPPDSIKDTKSDIIFFQTSVPGHDNKMQFESS |
| | | SYEGYFLASEKERDLFKLILKKEDELGDRSIMFTVQNED |
| | | (SEQ ID NO: 252) |
| 86 | N91S, R104H | YFGKLESKLSVIRNLNDQVLFIDQGNRPLFEDMTDSDM |
| | | RDNAPRTIFIISMYKDSQPRGMAVTISVKSEKISTLSSEN |
| | | KIISFKEMNPPDSIKDTKSDIIFFQHSVPGHDNKMQFESS |
| | | SYEGYFLASEKERDLFKLILKKEDELGDRSIMFTVQNED |
| | | (SEQ ID NO: 253) |
| 87 | K53T, D54H, S55V, T63A, | YFGKLESKLSVIRNLNDQVLFIDQGNRPLFEDMTDSDM |
| | N91K | RDNAPRTIFIISMYTHVQPRGMAVAISVKSEKISTLSSEN |
| | | KIISFKEMNPPDKIKDTKSDIIFFQRSVPGHDNKMQFESS |
| | | SYEGYFLASEKERDLFKLILKKEDELGDRSIMFTVQNED |
| | | (SEQ ID NO: 254) |
| 88 | E6G, D54A, S55K, T63S, | YFGKLGSKLSVIRNLNDQVLFIDQGNRPLFEDMTDSDM |
| | N91K, Q114R | RDNAPRTIFIISMYKAKQPRGMAVSISVKSEKISTLSSEN |
| | | KIISFKEMNPPDKIKDTKSDIIFFQRSVPGHDNKMRFESS |
| | | SYEGYFLASEKERDLFKLILKKEDELGDRSIMFTVQNED |
| | | (SEQ ID NO: 255) |
| 89 | E6N, D54N, S55N, N91H, | YFGKLNSKLSVIRNLNDQVLFIDQGNRPLFEDMTDSDM |
| | R104P | RDNAPRTIFIISMYKNNQPRGMAVTISVKSEKISTLSSEN |
| | | KIISFKEMNPPDHIKDTKSDIIFFQPSVPGHDNKMQFESS |
| | | SYEGYFLASEKERDLFKLILKKEDELGDRSIMFTVQNED |
| | | (SEQ ID NO: 256) |
| 90 | E6T, D54P, S55K, R104S | YFGKLTSKLSVIRNLNDQVLFIDQGNRPLFEDMTDSDM |
| | | RDNAPRTIFIISMYKPKQPRGMAVTISVKSEKISTLSSEN |
| | | KIISFKEMNPPDNIKDTKSDIIFFQSSVPGHDNKMQFESS |
| | | SYEGYFLASEKERDLFKLILKKEDELGDRSIMFTVQNED |
| | | (SEQ ID NO: 257) |
| 91 | G3H, E6Q, K53N, D54S, | YFHKLQSKLSVIRNLNDQVLFIDQGNRPLFEDMTDSDM |
| | S55P, T63A, N91I, R104S | RDNAPRTIFIISMYNSPQPRGMAVAISVKSEKISTLSSEN |
| | | KIISFKEMNPPDIIKDTKSDIIFFQSSVPGHDNKMQFESSS |
| | | YEGYFLASEKERDLFKLILKKEDELGDRSIMFTVQNED |
| | | (SEQ ID NO: 258) |
| 92 | G3I, E6D, D54A, S55F, | YFIKLDSKLSVIRNLNDQVLFIDQGNRPLFEDMTDSDM |
| | N91Y, R104H | RDNAPRTIFIISMYKAFQPRGMAVTISVKSEKISTLSSEN |
| | | KIISFKEMNPPDYIKDTKSDIIFFQHSVPGHDNKMQFESS |
| | | SYEGYFLASEKERDLFKLILKKEDELGDRSIMFTVQNED |
| | | (SEQ ID NO: 259) |
| 93 | G3I, E6Q, N91M, R104Y | YFIKLQSKLSVIRNLNDQVLFIDQGNRPLFEDMTDSDM |
| | | RDNAPRTIFIISMYKDSQPRGMAVTISVKSEKISTLSSEN |
| | | KIISFKEMNPPDMIKDTKSDIIFFQYSVPGHDNKMQFESS |
| | | SYEGYFLASEKERDLFKLILKKEDELGDRSIMFTVQNED |
| | | (SEQ ID NO: 260) |
| 94 | G3L, E6N, 122M, T63V, | YFLKLNSKLSVIRNLNDQVLFMDQGNRPLFEDMTDSD |
| | N91T, R104P | MRDNAPRTIFIISMYKDSQPRGMAVVISVKSEKISTLSSE |
| | | NKIISFKEMNPPDTIKDTKSDIIFFQPSVPGHDNKMQFES |
| | | SSYEGYFLASEKERDLFKLILKKEDELGDRSIMFTVQNE |
| | | D (SEQ ID NO: 261) |
| 95 | G3L, E6Q, T63H, N91M, | YFLKLQSKLSVIRNLNDQVLFIDQGNRPLFEDMTDSDM |
| | R104H | RDNAPRTIFIISMYKDSQPRGMAVHISVKSEKISTLSSEN |
| | | KIISFKEMNPPDMIKDTKSDIIFFQHSVPGHDNKMQFESS |
| | | SYEGYFLASEKERDLFKLILKKEDELGDRSIMFTVQNED |
| | | (SEQ ID NO: 262) |
| 96 | G3L, E6Q, T63S, N91L, | YFLKLQSKLSVIRNLNDQVLFIDQGNRPLFEDMTDSDM |
| | R104P | RDNAPRTIFIISMYKDSQPRGMAVSISVKSEKISTLSSEN |
| | | KIISFKEMNPPDLIKDTKSDIIFFQPSVPGHDNKMQFESS |
| | | SYEGYFLASEKERDLFKLILKKEDELGDRSIMFTVQNED |
| | | (SEQ ID NO: 263) |
| 97 | G3L, E6Q, T63S, N91L, | YFLKLQSKLSVIRNLNDQVLFIDQGNRPLFEDMTDSDM |
| | R104T | RDNAPRTIFIISMYKDSQPRGMAVSISVKSEKISTLSSEN |
| | | KIISFKEMNPPDLIKDTKSDIIFFQTSVPGHDNKMQFESS |
| | | SYEGYFLASEKERDLFKLILKKEDELGDRSIMFTVQNED |
| | | (SEQ ID NO: 264) |
| 98 | G3L, E6Q, D54Y, S55P, | YFLKLQSKLSVIRNLNDQVLFIDQGNRPLFEDMTDSDM |
| | T63S, N91S, R104Q | RDNAPRTIFIISMYKYPQPRGMAVSISVKSEKISTLSSEN |
| | | KIISFKEMNPPDSIKDTKSDIIFFQQSVPGHDNKMQFESS |
| | | SYEGYFLASEKERDLFKLILKKEDELGDRSIMFTVQNED |
| | | (SEQ ID NO: 265) |
| 99 | G3L, E6S, D54T, T63S, | YFLKLSSKLSVIRNLNDQVLFIDQGNRPLFEDMTDSDM |
| | N91Y, R104Q | RDNAPRTIFIISMYKTSQPRGMAVSISVKSEKISTLSSEN |
| | | KIISFKEMNPPDYIKDTKSDIIFFQQSVPGHDNKMQFESS |
| | | SYEGYFLASEKERDLFKLILKKEDELGDRSIMFTVQNED |
| | | (SEQ ID NO: 266) |
| 100 | G3L, E6Y, D54T, T63S, | YFLKLYSKLSVIRNLNDQVLFIDQGNRPLFEDMTDSDM |
| | N91Y, R104Q | RDNAPRTIFIISMYKTSQPRGMAVSISVKSEKISTLSSEN |
| | | KIISFKEMNPPDYIKDTKSDIIFFQQSVPGHDNKMQFESS |
| | | SYEGYFLASEKERDLFKLILKKEDELGDRSIMFTVQNED |
| | | (SEQ ID NO: 267) |
| 101 | G3N, E6S, K53Q, D54W, | YFNKLSSKLSVIRNLNDQVLFIDQGNRPLFEDMTDSDM |
| | S55R, N91T, R104T | RDNAPRTIFIISMYQWRQPRGMAVTISVKSEKISTLSSEN |
| | | KIISFKEMNPPDTIKDTKSDIIFFQTSVPGHDNKMQFESS |
| | | SYEGYFLASEKERDLFKLILKKEDELGDRSIMFTVQNED |
| | | (SEQ ID NO: 268) |
| 102 | G3N, E6Y, K53Q, D54W, | YFNKLYSKLSVIRNLNDQVLFIDQGNRPLFEDMTDSDM |
| | S55R, N91T, R104P | RDNAPRTIFIISMYQWRQPRGMAVTISVKSEKISTLSSEN |
| | | KIISFKEMNPPDTIKDTKSDIIFFQPSVPGHDNKMQFESS |
| | | SYEGYFLASEKERDLFKLILKKEDELGDRSIMFTVQNED |
| | | (SEQ ID NO: 269) |
| 103 | G3P, E6D, K53T, D54N, | YFPKLDSKLSVIRNLNDQVLFIDQGNRPLFEDMTDSDM |
| | T63A, N91T, R104P | RDNAPRTIFIISMYTNSQPRGMAVAISVKSEKISTLSSEN |
| | | KIISFKEMNPPDTIKDTKSDIIFFQPSVPGHDNKMQFESS |
| | | SYEGYFLASEKERDLFKLILKKEDELGDRSIMFTVQNED |
| | | (SEQ ID NO: 270) |
| 104 | G3P, E6G, D54P, S55W, | YFPKLGSKLSVIRNLNDQVLFIDQGNRPLFEDMTDSDM |
| | N91E, R104K | RDNAPRTIFIISMYKPWQPRGMAVTISVKSEKISTLSSEN |
| | | KIISFKEMNPPDEIKDTKSDIIFFQKSVPGHDNKMQFESS |
| | | SYEGYFLASEKERDLFKLILKKEDELGDRSIMFTVQNED |
| | | (SEQ ID NO: 271) |
| 105 | G3P, E6G, D54T, S55W, | YFPKLGSKLSVIRNLNDQVLFIDQGNRPLFEDMTDSDM |
| | N91E, R104K | RDNAPRTIFIISMYKTWQPRGMAVTISVKSEKISTLSSEN |
| | | KIISFKEMNPPDEIKDTKSDIIFFQKSVPGHDNKMQFESS |
| | | SYEGYFLASEKERDLFKLILKKEDELGDRSIMFTVQNED |
| | | (SEQ ID NO: 272) |
| 106 | G3P, E6H, D54A, S55P, | YFPKLHSKLSVIRNLNDQVLFIDQGNRPLFEDMTDSDM |
| | Q56K, T63S, N91M | RDNAPRTIFIISMYKAPKPRGMAVSISVKSEKISTLSSEN |
| | | KIISFKEMNPPDMIKDTKSDIIFFQRSVPGHDNKMQFESS |
| | | SYEGYFLASEKERDLFKLILKKEDELGDRSIMFTVQNED |
| | | (SEQ ID NO: 273) |
| 107 | G3P, E6H, N91L | YFPKLHSKLSVIRNLNDQVLFIDQGNRPLFEDMTDSDM |
| | | RDNAPRTIFIISMYKDSQPRGMAVTISVKSEKISTLSSEN |
| | | KIISFKEMNPPDLIKDTKSDIIFFQRSVPGHDNKMQFESS |
| | | SYEGYFLASEKERDLFKLILKKEDELGDRSIMFTVQNED |
| | | (SEQ ID NO: 274) |
| 108 | G3P, E6I, K53N, D54W, | YFPKLISKLSVIRNLNDQVLFIDQGNRPLFEDMTDSDMR |
| | S55P, T63S, N91L, R104Q | DNAPRTIFIISMYNWPQPRGMAVSISVKSEKISTLSSENK |
| | | IISFKEMNPPDLIKDTKSDIIFFQQSVPGHDNKMQFESSS |
| | | YEGYFLASEKERDLFKLILKKEDELGDRSIMFTVQNED |
| | | (SEQ ID NO: 275) |
| 109 | G3P, E6K, D54R, S55P, | YFPKLKSKLSVIRNLNDQVLFIDQGNRPLFEDMTDSDM |
| | T63A, N91V | RDNAPRTIFIISMYKRPQPRGMAVAISVKSEKISTLSSEN |
| | | KIISFKEMNPPDVIKDTKSDIIFFQRSVPGHDNKMQFESS |
| | | SYEGYFLASEKERDLFKLILKKEDELGDRSIMFTVQNED |
| | | (SEQ ID NO: 276) |
| 110 | G3P, E6K, D54Y, S55P, | YFPKLKSKLSVIRNLNDQVLFIDQGNRPLFEDMTDSDM |
| | Q56H, T63S, N91S, R104Q | RDNAPRTIFIISMYKYPHPRGMAVSISVKSEKISTLSSEN |
| | | KIISFKEMNPPDSIKDTKSDIIFFQQSVPGHDNKMQFESS |
| | | SYEGYFLASEKERDLFKLILKKEDELGDRSIMFTVQNED |
| | | (SEQ ID NO: 277) |
| 111 | G3P, E6K, D54Y, S55P, | YFPKLKSKLSVIRNLNDQVLFIDQGNRPLFEDMTDSDM |
| | T63S, N91S, R104Q | RDNAPRTIFIISMYKYPQPRGMAVSISVKSEKISTLSSEN |
| | | KIISFKEMNPPDSIKDTKSDIIFFQQSVPGHDNKMQFESS |
| | | SYEGYFLASEKERDLFKLILKKEDELGDRSIMFTVQNED |
| | | (SEQ ID NO: 278) |
| 112 | G3P, E6K, D54Y, S55P, | YFPKLKSKLSVIRNLNDQVLFIDQGNRPLFEDMTDSDM |
| | T63S, N91S, R104Q, | RDNAPRTIFIISMYKYPQPRGMAVSISVKSEKISTLSSEN |
| | Q154R | KIISFKEMNPPDSIKDTKSDIIFFQQSVPGHDNKMQFESS |
| | | SYEGYFLASEKERDLFKLILKKEDELGDRSIMFTVRNED |
| | | (SEQ ID NO: 279) |
| 113 | G3P, E6K, K53M, D54W, | YFPKLKSKLSVIRNLNDQVLFIDQGNRPLFEDMTDSDM |
| | S55N, T63S, N91I | RDNAPRTIFIISMYMWNQPRGMAVSISVKSEKISTLSSE |
| | | NKIISFKEMNPPDIIKDTKSDIIFFQRSVPGHDNKMQFES |
| | | SSYEGYFLASEKERDLFKLILKKEDELGDRSIMFTVQNE |
| | | D (SEQ ID NO: 280) |
| 114 | G3P, E6K, K53N, D54S, | YFPKLKSKLSVIRNLNDQVLFIDQGNRPLFEDMTDSDM |
| | S55P, T63S, N91S, R104Q | RDNAPRTIFIISMYNSPQPRGMAVSISVKSEKISTLSSEN |
| | | KIISFKEMNPPDSIKDTKSDIIFFQQSVPGHDNKMQFESS |
| | | SYEGYFLASEKERDLFKLILKKEDELGDRSIMFTVQNED |
| | | (SEQ ID NO: 281) |
| 115 | G3P, E6K, K53T, D54Y, | YFPKLKSKLSVIRNLNDQVLFIDQGNRPLFEDMTDSDM |
| | S55V, T63I, N91I | RDNAPRTIFIISMYTYVQPRGMAVIISVKSEKISTLSSEN |
| | | KIISFKEMNPPDIIKDTKSDIIFFQRSVPGHDNKMQFESSS |
| | | YEGYFLASEKERDLFKLILKKEDELGDRSIMFTVQNED |
| | | (SEQ ID NO: 282) |
| 116 | G3P, E6N, D54Y, T63S, | YFPKLNSKLSVIRNLNDQVLFIDQGNRPLFEDMTDSDM |
| | N91S, R104Q | RDNAPRTIFIISMYKYSQPRGMAVSISVKSEKISTLSSEN |
| | | KIISFKEMNPPDSIKDTKSDIIFFQQSVPGHDNKMQFESS |
| | | SYEGYFLASEKERDLFKLILKKEDELGDRSIMFTVQNED |
| | | (SEQ ID NO: 283) |
| 117 | G3P, E6R, D54W, S55Q, | YFPKLRSKLSVIRNLNDQVLFIDQGNRPLFEDMTDSDM |
| | T63S, N91T, R104H | RDNAPRTIFIISMYKWQQPRGMAVSISVKSEKISTLSSEN |
| | | KIISFKEMNPPDTIKDTKSDIIFFQHSVPGHDNKMQFESS |
| | | SYEGYFLASEKERDLFKLILKKEDELGDRSIMFTVQNED |
| | | (SEQ ID NO: 284) |
| 118 | G3P, E6R, K53T, D54Y, | YFPKLRSKLSVIRNLNDQVLFIDQGNRPLFEDMTDSDM |
| | S55I, T63V, N91G, R104K | RDNAPRTIFIISMYTYIQPRGMAVVISVKSEKISTLSSEN |
| | | KIISFKEMNPPDGIKDTKSDIIFFQKSVPGHDNKMQFESS |
| | | SYEGYFLASEKERDLFKLILKKEDELGDRSIMFTVQNED |
| | | (SEQ ID NO: 285) |
| 119 | G3P, E6S, K53N, D54Y, | YFPKLSSKLSVIRNLNDQVLFIDQGNRPLFEDMTDSDM |
| | S55P, T63I, N91K, R104H | RDNAPRTIFIISMYNYPQPRGMAVIISVKSEKISTLSSEN |
| | | KIISFKEMNPPDKIKDTKSDIIFFQHSVPGHDNKMQFESS |
| | | SYEGYFLASEKERDLFKLILKKEDELGDRSIMFTVQNED |
| | | (SEQ ID NO: 286) |
| 120 | G3P, E6S, K53R, D54Y, | YFPKLSSKLSVIRNLNDQVLFIDQGNRPLFEDMTDSDM |
| | S55P, T63I, N91K | RDNAPRTIFIISMYRYPQPRGMAVIISVKSEKISTLSSENK |
| | | IISFKEMNPPDKIKDTKSDIIFFQRSVPGHDNKMQFESSS |
| | | YEGYFLASEKERDLFKLILKKEDELGDRSIMFTVQNED |
| | | (SEQ ID NO: 287) |
| 121 | G3P, E6V, K53A, D54W, | YFPKLVSKLSVIRNLNDQVLFIDQGNRPLFEDMTDSDM |
| | S55P, T63S, N91L, R104K | RDNAPRTIFIISMYAWPQPRGMAVSISVKSEKISTLSSEN |
| | | KIISFKEMNPPDLIKDTKSDIIFFQKSVPGHDNKMQFESS |
| | | SYEGYFLASEKERDLFKLILKKEDELGDRSIMFTVQNED |
| | | (SEQ ID NO: 288) |
| 122 | G3P, E6V, K53A, D54W, | YFPKLVSKLSVIRNLNDQVLFIDQGNRPLFEDMTDSDM |
| | S55P, T63S, N91L, R104K, | RDNAPRTIFIISMYAWPQPRGMAVSISVKSEKISTLSSEN |
| | Q154R | KIISFKEMNPPDLIKDTKSDIIFFQKSVPGHDNKMQFESS |
| | | SYEGYFLASEKERDLFKLILKKEDELGDRSIMFTVRNED |
| | | (SEQ ID NO: 289) |
| 123 | G3Q, E6D, K53S, D54I, | YFQKLDSKLSVIRNLNDQVLFIDQGNRPLFEDMTDSDM |
| | S55E, T63S, N91S, R104Q | RDNAPRTIFIISMYSIEQPRGMAVSISVKSEKISTLSSENK |
| | | IISFKEMNPPDSIKDTKSDIIFFQQSVPGHDNKMQFESSS |
| | | YEGYFLASEKERDLFKLILKKEDELGDRSIMFTVQNED |
| | | (SEQ ID NO: 290) |
| 124 | G3Q, E6G, D54N, S55H, | YFQKLGSKLSVIRNLNDQVLFIDQGNRPLFEDMTDSDM |
| | T63V, N91M, R104T | RDNAPRTIFIISMYKNHQPRGMAVVISVKSEKISTLSSEN |
| | | KIISFKEMNPPDMIKDTKSDIIFFQTSVPGHDNKMQFESS |
| | | SYEGYFLASEKERDLFKLILKKEDELGDRSIMFTVQNED |
| | | (SEQ ID NO: 291) |
| 125 | G3Q, E6H, D54N, S55I, | YFQKLHSKLSVIRNLNDQVLFIDQGNRPLFEDMTDSDM |
| | T63S, N91V | RDNAPRTIFIISMYKNIQPRGMAVSISVKSEKISTLSSEN |
| | | KIISFKEMNPPDVIKDTKSDIIFFQRSVPGHDNKMQFESS |
| | | SYEGYFLASEKERDLFKLILKKEDELGDRSIMFTVQNED |
| | | (SEQ ID NO: 292) |
| 126 | G3Q, E6H, K53T, D54S, | YFQKLHSKLSVIRNLNDQVLFIDQGNRPLFEDMTDSDM |
| | S55Y, N91Y, R104K | RDNAPRTIFIISMYTSYQPRGMAVTISVKSEKISTLSSEN |
| | | KIISFKEMNPPDYIKDTKSDIIFFQKSVPGHDNKMQFESS |
| | | SYEGYFLASEKERDLFKLILKKEDELGDRSIMFTVQNED |
| | | (SEQ ID NO: 293) |
| 127 | G3Q, E6H, S7Y, D23A, | YFQKLHYKLSVIRNLNDQVLFIAQGNSPLFEDMTDSDM |
| | R27S, D54S, S55R, T63F, | RDNAPRTIFIISMYKSRQPRGMAVFISVKSEKISTLSSEN |
| | N91F, R104Q | KIISFKEMNPPDFIKDTKSDIIFFQQSVPGHDNKMQFESS |
| | | SYEGYFLASEKERDLFKLILKKEDELGDRSIMFTVQNED |
| | | (SEQ ID NO: 294) |
| 128 | G3Q, E6K, D54S, S55P, | YFQKLKSKLSVIRNLNDQVLFIDQGNRPLFEDMTDSDM |
| | T63S, N91S, R104Q | RDNAPRTIFIISMYKSPQPRGMAVSISVKSEKISTLSSEN |
| | | KIISFKEMNPPDSIKDTKSDIIFFQQSVPGHDNKMQFESS |
| | | SYEGYFLASEKERDLFKLILKKEDELGDRSIMFTVQNED |
| | | (SEQ ID NO: 295) |
| 129 | G3Q, E6N | YFQKLNSKLSVIRNLNDQVLFIDQGNRPLFEDMTDSDM |
| | | RDNAPRTIFIISMYKDSQPRGMAVTISVKSEKISTLSSEN |
| | | KIISFKEMNPPDNIKDTKSDIIFFQRSVPGHDNKMQFESS |
| | | SYEGYFLASEKERDLFKLILKKEDELGDRSIMFTVQNED |
| | | (SEQ ID NO: 296) |
| 130 | G3Q, E6N, D54Y, S55P, | YFQKLNSKLSVIRNLNDQVLFIDQGNRPLFEDMTDSDM |
| | T63S, N91S, R104H | RDNAPRTIFIISMYKYPQPRGMAVSISVKSEKISTLSSEN |
| | | KIISFKEMNPPDSIKDTKSDIIFFQHSVPGHDNKMQFESS |
| | | SYEGYFLASEKERDLFKLILKKEDELGDRSIMFTVQNED |
| | | (SEQ ID NO: 297) |
| 131 | G3Q, E6N, K53T, D54A, | YFQKLNSKLSVIRNLNDQVLFIDQGNRPLFEDMTDSDM |
| | N91K, R104P | RDNAPRTIFIISMYTASQPRGMAVTISVKSEKISTLSSEN |
| | | KIISFKEMNPPDKIKDTKSDIIFFQPSVPGHDNKMQFESS |
| | | SYEGYFLASEKERDLFKLILKKEDELGDRSIMFTVQNED |
| | | (SEQ ID NO: 298) |
| 132 | G3Q, E6Q, K53T, D54W, | YFQKLQSKLSVIRNLNDQVLFIDQGNRPLFEDMTDSDM |
| | S55F, N91Y | RDNAPRTIFIISMYTWFQPRGMAVTISVKSEKISTLSSEN |
| | | KIISFKEMNPPDYIKDTKSDIIFFQRSVPGHDNKMQFESS |
| | | SYEGYFLASEKERDLFKLILKKEDELGDRSIMFTVQNED |
| | | (SEQ ID NO: 299) |
| 133 | G3Q, E6S, S55V, T63A, | YFQKLSSKLSVIRNLNDQVLFIDQGNRPLFEDMTDSDM |
| | N91T | RDNAPRTIFIISMYKDVQPRGMAVAISVKSEKISTLSSEN |
| | | KIISFKEMNPPDTIKDTKSDIIFFQRSVPGHDNKMQFESS |
| | | SYEGYFLASEKERDLFKLILKKEDELGDRSIMFTVQNED |
| | | (SEQ ID NO: 300) |
| 134 | G3R, E6D, D54A, S55Q, | YFRKLDSKLSVIRNLNDQVLFIDQGNRPLFEDMTDSDM |
| | T63S, N91K, R104P | RDNAPRTIFIISMYKAQQPRGMAVSISVKSEKISTLSSEN |
| | | KIISFKEMNPPDKIKDTKSDIIFFQPSVPGHDNKMQFESS |
| | | SYEGYFLASEKERDLFKLILKKEDELGDRSIMFTVQNED |
| | | (SEQ ID NO: 301) |
| 135 | G3R, L5W, E6H, D54Y, | YFRKWHSKLSVIRNLNDQVLFIDQGNRPLFEDMTDSD |
| | Q56H, N91R | MRDNAPRTIFIISMYKYSHPRGMAVTISVKSEKISTLSSE |
| | | NKIISFKEMNPPDRIKDTKSDIIFFQRSVPGHDNKMQFES |
| | | SSYEGYFLASEKERDLFKLILKKEDELGDRSIMFTVQNE |
| | | D (SEQ ID NO: 302) |
| 136 | G3S, E6G, D54V, S55K, | YFSKLGSKLSVIRNLNDQVLFIDQGNRPLFEDMTDSDM |
| | T63S, N91Y, R104T | RDNAPRTIFIISMYKVKQPRGMAVSISVKSEKISTLSSEN |
| | | KIISFKEMNPPDYIKDTKSDIIFFQTSVPGHDNKMQFESS |
| | | SYEGYFLASEKERDLFKLILKKEDELGDRSIMFTVQNED |
| | | (SEQ ID NO: 303) |
| 137 | G3S, E6H, K53Y, D54P, | YFSKLHSKLSVIRNLNDQVLFIDQGNRPLFEDMTDSDM |
| | S55Y, T63S, N91A, R104S | RDNAPRTIFIISMYYPYQPRGMAVSISVKSEKISTLSSEN |
| | | KIISFKEMNPPDAIKDTKSDIIFFQSSVPGHDNKMQFESS |
| | | SYEGYFLASEKERDLFKLILKKEDELGDRSIMFTVQNED |
| | | (SEQ ID NO: 304) |
| 138 | G3S, E6Q, K53N, D54T, | YFSKLQSKLSVIRNLNDQVLFIDQGNRPLFEDMTDSDM |
| | S55N, T63A, 180T, N91S | RDNAPRTIFIISMYNTNQPRGMAVAISVKSEKISTLSSEN |
| | | KTISFKEMNPPDSIKDTKSDIIFFQRSVPGHDNKMQFESS |
| | | SYEGYFLASEKERDLFKLILKKEDELGDRSIMFTVQNED |
| | | (SEQ ID NO: 305) |
| 139 | G3S, E6Q, K53S, D54Y, | YFSKLQSKLSVIRNLNDQVLFIDQGNRPLFEDMTDSDM |
| | S55H, N91L, R104Q | RDNAPRTIFIISMYSYHQPRGMAVTISVKSEKISTLSSEN |
| | | KIISFKEMNPPDLIKDTKSDIIFFQQSVPGHDNKMQFESS |
| | | SYEGYFLASEKERDLFKLILKKEDELGDRSIMFTVQNED |
| | | (SEQ ID NO: 306) |
| 140 | G3T, E6Q, K53G, D54G, | YFTKLQSKLSVIRNLNDQVLFIDQGNRPLFEDMTDSDM |
| | S55H, T63A, S65T, N91R, | RDNAPRTIFIISMYGGHQPRGMAVAITVKSEKISTLSSEN |
| | R104Q | KIISFKEMNPPDRIKDTKSDIIFFQQSVPGHDNKMQFESS |
| | | SYEGYFLASEKERDLFKLILKKEDELGDRSIMFTVQNED |
| | | (SEQ ID NO: 307) |
| 141 | G3V, E6Q, T34P, D54M, | YFVKLQSKLSVIRNLNDQVLFIDQGNRPLFEDMPDSDM |
| | S55F, N91R, R104H | RDNAPRTIFIISMYKMFQPRGMAVTISVKSEKISTLSSEN |
| | | KIISFKEMNPPDRIKDTKSDIIFFQHSVPGHDNKMQFESS |
| | | SYEGYFLASEKERDLFKLILKKEDELGDRSIMFTVQNED |
| | | (SEQ ID NO: 308) |
| 142 | G3Y, E6N, N91Y, R104S | YFYKLNSKLSVIRNLNDQVLFIDQGNRPLFEDMTDSDM |
| | | RDNAPRTIFIISMYKDSQPRGMAVTISVKSEKISTLSSEN |
| | | KIISFKEMNPPDYIKDTKSDIIFFQSSVPGHDNKMQFESS |
| | | SYEGYFLASEKERDLFKLILKKEDELGDRSIMFTVQNED |
| | | (SEQ ID NO: 309) |
| 143 | G3Y, E6S, K53S, D54W, | YFYKLSSKLSVIRNLNDQVLFIDQGNRPLFEDMTDSDM |
| | S55L, T63S, N91I | RDNAPRTIFIISMYSWLQPRGMAVSISVKSEKISTLSSEN |
| | | KIISFKEMNPPDIIKDTKSDIIFFQRSVPGHDNKMQFESSS |
| | | YEGYFLASEKERDLFKLILKKEDELGDRSIMFTVQNED |
| | | (SEQ ID NO: 310) |
| 144 | G3Y, E6S, K53S, D54W, | YFYKLSSKLSVIRNLNDQVLFIDQGNRPLFEDMTDSDM |
| | S55L, N91I | RDNAPRTIFIISMYSWLQPRGMAVTISVKSEKISTLSSEN |
| | | KIISFKEMNPPDIIKDTKSDIIFFQRSVPGHDNKMQFESSS |
| | | YEGYFLASEKERDLFKLILKKEDELGDRSIMFTVQNED |
| | | (SEQ ID NO: 311) |

TABLE 3
Mouse IL-18 Variants.

| | Amino Acid | |
|---|---|---|
| No | Substitutions | Amino Acid Sequence |
| 1 | G3T, H6K, | NFTRLKSTTAVIRNINDQVLFVDKRQPVFEDMTDIDQSASEPQTRLII |
| | K52A | YMYADSEVRGLAVTLSVKDSKMSTLSSKNKIISFEEMDPPENIDDIQ |
| | | SDLIFFQKRVPGHNKMEFESSLYEGHFLACQKEDDAFKLILKKKDE |
| | | NGDKSVMFTLTNLHQS |
| 2 | G3V, H6K, | NFVRLKSTTAVIRNINDQVLFVDKRQPVFEDMTDIDQSASEPQTRLII |
| | K52A | YMYADSEVRGLAVTLSVKDSKMSTLSSKNKIISFEEMDPPENIDDIQ |
| | | SDLIFFQKRVPGHNKMEFESSLYEGHFLACQKEDDAFKLILKKKDE |
| | | NGDKSVMFTLTNLHQS |
| 3 | G3T, K52A | NFTRLHSTTAVIRNINDQVLFVDKRQPVFEDMTDIDQSASEPQTRLII |
| | | YMYADSEVRGLAVTLSVKDSKMSTLSSKNKIISFEEMDPPENIDDIQ |
| | | SDLIFFQKRVPGHNKMEFESSLYEGHFLACQKEDDAFKLILKKKDE |
| | | NGDKSVMFTLTNLHQS |
| 4 | G3V, K52A | NFVRLHSTTAVIRNINDQVLFVDKRQPVFEDMTDIDQSASEPQTRLII |
| | | YMYADSEVRGLAVTLSVKDSKMSTLSSKNKIISFEEMDPPENIDDIQ |
| | | SDLIFFQKRVPGHNKMEFESSLYEGHFLACQKEDDAFKLILKKKDE |
| | | NGDKSVMFTLTNLHQS |
| 5 | C7S/C75T/C12 | NFGRLHSTTAVIRNINDQVLFVDKRQPVFEDMTDIDQSASEPQTRLII |
| | 5T | YMYKDSEVRGLAVTLSVKDSKMSTLSTKNKIISFEEMDPPENIDDIQ |
| | | SDLIFFQKRVPGHNKMEFESSLYEGHFLATQKEDDAFKLILKKKDE |
| | | NGDKSVMFTLTNLHQS |
| 6 | C7W/C75G/C1 | NFGRLHWTTAVIRNINDQVLFVDKRQPVFEDMTDIDQSASEPQTRLI |
| | 25D | IYMYKDSEVRGLAVTLSVKDSKMSTLSGKNKIISFEEMDPPENIDDI |
| | | QSDLIFFQKRVPGHNKMEFESSLYEGHFLADQKEDDAFKLILKKKD |
| | | ENGDKSVMFTLTNLHQS |
| 7 | C7W/C75T/C1 | NFGRLHWTTAVIRNINDQVLFVDKRQPVFEDMTDIDQSASEPQTRLI |
| | 25D | IYMYKDSEVRGLAVTLSVKDSKMSTLSTKNKIISFEEMDPPENIDDI |
| | | QSDLIFFQKRVPGHNKMEFESSLYEGHFLADQKEDDAFKLILKKKD |
| | | ENGDKSVMFTLTNLHQS |
| 8 | C7W/C75A/C1 | NFGRLHWTTAVIRNINDQVLFVDKRQPVFEDMTDIDQSASEPQTRLI |
| | 25D | IYMYKDSEVRGLAVTLSVKDSKMSTLSAKNKIISFEEMDPPENIDDI |
| | | QSDLIFFQKRVPGHNKMEFESSLYEGHFLADQKEDDAFKLILKKKD |
| | | ENGDKSVMFTLTNLHQS |
| 9 | C7S/C75A/C12 | NFGRLHSTTAVIRNINDQVLFVDKRQPVFEDMTDIDQSASEPQTRLII |
| | 5V | YMYKDSEVRGLAVTLSVKDSKMSTLSAKNKIISFEEMDPPENIDDIQ |
| | | SDLIFFQKRVPGHNKMEFESSLYEGHFLAVQKEDDAFKLILKKKDE |
| | | NGDKSVMFTLTNLHQS |
| 10 | C7W/C75A/C1 | NFGRLHWTTAVIRNINDQVLFVDKRQPVFEDMTDIDQSASEPQTRLI |
| | 25T | IYMYKDSEVRGLAVTLSVKDSKMSTLSAKNKIISFEEMDPPENIDDI |
| | | QSDLIFFQKRVPGHNKMEFESSLYEGHFLATQKEDDAFKLILKKKD |
| | | ENGDKSVMFTLTNLHQS |
| 11 | C7S/C75S/C12 | NFGRLHSTTAVIRNINDQVLFVDKRQPVFEDMTDIDQSASEPQTRLII |
| | 5S | YMYKDSEVRGLAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |
| 12 | G3T, L59K | NFTRLHSTTAVIRNINDQVLFVDKRQPVFEDMTDIDQSASEPQTRLII |
| | | YMYKDSEVRGKAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |
| 13 | G3V, L59K | NFVRLHSTTAVIRNINDQVLFVDKRQPVFEDMTDIDQSASEPQTRLII |
| | | YMYKDSEVRGKAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |
| 14 | G3T, H6K, | NFTRLKSTTAVIRNINDQVLFVDKRQPVFEDMTDIDQSASEPQTRLII |
| | L59K | YMYKDSEVRGKAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |
| 15 | G3V, H6K, | NFVRLKSTTAVIRNINDQVLFVDKRQPVFEDMTDIDQSASEPQTRLII |
| | L59K | YMYKDSEVRGKAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |
| 16 | G3V, L59M | NFVRLHSTTAVIRNINDQVLFVDKRQPVFEDMTDIDQSASEPQTRLII |
| | | YMYKDSEVRGMAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |
| 17 | G3V, L59R | NFVRLHSTTAVIRNINDQVLFVDKRQPVFEDMTDIDQSASEPQTRLII |
| | | YMYKDSEVRGRAVTLSVKDSKMSTLSSKNKIISFEEMDPPENIDDIQ |
| | | SDLIFFQKRVPGHNKMEFESSLYEGHFLASQKEDDAFKLILKKKDEN |
| | | GDKSVMFTLTNLHQS |

TABLE 4
Comparator IL-18 Variants. Comparators 1-3 are human IL-18 variants and
Comparators 4 and 5 are mouse IL-18 variants

| | Amino Acid | |
|---|---|---|
| ID | Substitutions | Amino Acid Sequence |
| Comparator 1 | M51K, K53S, Q56L, | YFGKLESKLSVIRNLNDQVLFIDQGNRPLFEDMTDSDC |
| | P57A, M60L, S105D, | RDNAPRTIFIISKYSDSLARGLAVTISVKCEKISTLSCEN |
| | D110S, N111R | KIISFKEMNPPDNIKDTKSDIIFFQRDVPGHSRKMQFES |
| | | SSYEGYFLACEKERDLFKLILKKEDELGDRSIMFTVQN |
| | | ED |
| Comparator 2 | M51K, K53S, Q56R, | YFGKLESKLSVIRNLNDQVLFIDQGNRPLFEDMTDSDC |
| | P57A, M60L, | RDNAPRTIFIISKYSDSRARGLAVTISVKCEKISTLSCEN |
| | Q103A, D110G, | KIISFKEMNPPDNIKDTKSDIIFFARSVPGHGRKTQFESS |
| | N111R, M113T | SYEGYFLACEKERDLFKLILKKEDELGDRSIMFTVQNE |
| | | D |
| Comparator 3 | M51K, K53G, Q56G, | YFGKLESKLSVIRNLNDQVLFIDQGNRPLFEDMTDSDC |
| | P57A, M60L, | RDNAPRTIFIISKYGDSGARGLAVTISVKCEKISTLSCEN |
| | Q103E, S105D, | KIISFKEMNPPDNIKDTKSDIIFFERDVPGHSGKVQFESS |
| | D110S, N111G, | SYEGYFLACEKERDLFKLILKKEDELGDRSIMFTVQNE |
| | M113V | D |
| Comparator 4 | L59K | ASEPQTRLIIYGYADSRVRGKAVTLSVKDSKMSTLSCK |
| | M50G, K52A, E55R, | NKIISFEEMDPPENIDDIQSDLIFFQKRVPGHNKMEFESS |
| | | LYEGHFLACQKEDDAFKLILKKKDENGDKSVMFTLTNLHQS |
| | | NFGRLHCTTAVIRNINDQVLFVDKRQPVFEDMTDIDQS |
| Comparator 5 | NIH, M50A, K52G, | HFGRLHCTTAVIRNINDQVLFVDKRQPVFEDMTDIDQS |
| | E55R, V56A, L59K | ASEPQTRLIIYAYGDSRARGKAVTLSVKDSKMSTLSCK |
| | | NKIISFEEMDPPENIDDIQSDLIFFQKRVPGHNKMEFESS |
| | | LYEGHFLACQKEDDAFKLILKKKDENGDKSVMFTLTN |
| | | LHQS |

It is noted that all of the IL-18 variant sequences of Table 1 and Table 2 comprise all four cysteine substitutions described above (i.e., C38M, C68S, C76S, and C127S). However, IL-18 variant sequences of Table 1 and Table 2 without the cysteine substitutions are also envisioned herein.

Moreover, the IL-18 variant sequences of Tables 1-4 do not comprise an N-terminal methionine. In certain embodiments, the −18 variant sequences of Tables 1-4 comprise an N-terminal methionine amino acid.

In certain embodiments, the IL-18 polypeptide of the disclosure comprises an amino acid sequence of any one of the TL-18 polypeptide amino acid sequences of Table 1 or Table 2.

In certain embodiments, the IL-18 polypeptide of the disclosure comprises an amino acid sequence with at least 80% identity to any one of the IL-18 polypeptide amino acid sequences of Table 1 or Table 2.

### Linkers

The design of the peptide linkers connecting the IL-18 variants to other polypeptide (e.g., antibody Fc domains or serum albumin) are flexible linkers generally composed of small, non-polar or polar residues such as, e.g., Gly, Ser and Thr. A particularly exemplary linker connecting the variable domains of the scFv moieties is the (Gly₄Ser)₄ linker, where 4 is the exemplary number of repeats of the motif. In certain embodiments, the linker comprises the amino acid sequence GGSGGGGSGGGSGGGGSGGGGSGGGSGG, GGGGSGGGGSGGGGS, or GGGSGGGGSG. In certain embodiments, the linker comprises the amino acid sequence GGSGGGGSGG, GGSGG, or GGS.

Other exemplary linkers include, but are not limited to the following amino acid sequences: GGG; DGGGS; TGEKP (Liu et al, Proc. Natl. Acad. Sci. 94: 5525-5530 (1997)); GGRR; (GGGGS)ₙ wherein n=1, 2, 3, 4 or 5 (Kim et al, Proc. Natl. Acad. Sci. 93: 1156-1160 (1996)); EGKSSGSGSESKVD (Chaudhary et al., Proc. Natl. Acad. Sci. 87: 1066-1070 (1990)); KESGSVSSEQLAQFRSLD (Bird et al., Science 242:423-426 (1988)), GGRRGGGS; LRQRDGERP; LRQKDGGGSERP; and GSTSGSGKPGSGEGSTKG (Cooper et al, Blood, 101(4): 1637-1644 (2003)). Alternatively, flexible linkers can be rationally designed using a computer program capable of modeling the

In certain embodiments, any one or more linkers present in the proteins of the disclosure are selected from artificial flexible polypeptides comprising amino acids selected from Gly (G), and/or Ser (S). In certain embodiments, the linker is comprised of polypeptide of the general formula (GGGS)n or (GGGGS)n or (SGGSGGG)ₙ or (GGSGGSG)ₙ wherein n is an integer from 1 to 10. In certain embodiments, each linker is a polypeptide comprising from about 1 to about 100 amino acids, such as about 1-50 amino acids, about 1-25 amino acids, about 1-15 amino acids, about 1-10 amino acids, about 4-24 amino acids, about 5-20 amino acids, about 5-15 amino acids, and about 5-10 amino acids. In certain embodiments, the linker is (GGGGS) n wherein n is 2 or 4. Any linker may further comprise amino acids such as, for example, Lys (K), Thr (T), Glu (E), and Asp (D).

In certain embodiments, the amino acid linker comprises (GGGGS)n, wherein n is an integer between 1 and 5. In certain embodiments, the amino acid linker comprises the amino acid sequence GGGGSGGGGSGGGGS or GGSGGGGSGGGSGGGGSGGGGSGGGSGG. In certain embodiments, the amino acid linker comprises the amino acid sequence GGS.

In certain embodiments, the linker may comprise one or more mucin proteins or mucin domains of proteins (e.g., any protein encoded for by a MUC gene (e.g., MUC1, MUC2, MUC3A, MUC3B, MUC4, MUCSAC, MUCSB, MUC6, MUC7, MUC8, MUC9, MUC11, MUC12, MUC13, MUC15, MUC16, MUC17, MUC19, MUC20, MUC21). Mucin domain proteins and polypeptides contain a high degree of glycosylation which structurally allows mucin proteins and other polypeptides comprising mucin domains to behave as stiffened random coils. The rod-like nature of the mucin domains can rigidly separate the bioactive protein (e.g., the IL-18 variant) from the fusion partner (e.g., Fc domain or serum albumin), and thereby be less susceptible to loss in activity either fusion partner.

Such mucin domain polypeptides useful in accordance with the disclosure are described in WO 2013/184939 and WO 2013/184938, incorporated herein by reference. These linkers are useful to provide optimal spacing between the polypeptides of the fusion proteins of the disclosure (e.g., between the ATF polypeptide and cytokine polypeptide) or for example, to provide an increase in half-life of the fusion protein as a whole regardless of location of the mucin domain in the fusion protein. For example, a mucin-domain may be present at the N-terminus or C-terminus of the fusion protein. Mucin domain polypeptide linkers may further be linked to the Fc region of an immunoglobulin polypeptide that may also function to increase half-life of the fusion protein of the invention as is described in WO 2013/184938.

### Purification Tags

Any of the proteins described herein (e.g., the IL-18 variants) can include one or more (e.g., two or more, three or more, four or more, five or more, six or more, or seven or more) purification tags, which facilitate the purification of the proteins described herein. In certain embodiments, the purification tag is an avi tag (GLNDIFEAQKIEWHE). In certain embodiments, the purification tag is a 6×His tag (HHHHHH). One or both of the avi tag and 6×His tag may be present on a single protein and in any order. In certain embodiments, the purification tag is linked to a protein described herein with a gly-ser linker. In certain embodiments, the gly-ser linker comprises GGS and/or GGSGGG. In certain embodiments, the purification is any one of GGSHHHHHHGGSGLNDIFEAQKIEWHE, GGSGGHHHHHHGGSGLNDIFEAQKIEWHE, and GGSGLNDIFEAQKIEWHEGGSHHHHHH.

### Expression of IL-18 Variants

In one aspect, polynucleotides or nucleic acids encoding the IL-18 variants disclosed herein are provided. Methods of making an IL-18 variant comprising expressing these polynucleotides are also provided.

Polynucleotides encoding the IL-18 variants disclosed herein are typically inserted in an expression vector for introduction into host cells that may be used to produce the desired quantity of the antigen binding proteins or fusion proteins. Accordingly, in certain aspects, the disclosure provides expression vectors comprising polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals.

In other embodiments, the IL-18 variants may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an IL-18 variant has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the IL-18 variants, and assayed for protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for IL-18 variant expression is of mammalian origin. Those skilled in the art can determine particular host cell lines which are best suited for the desired gene product to be expressed therein. Exemplary host cell lines include, but are not limited to, DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the IL-18 variants featured in the invention can also be expressed non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

### Methods of Administering IL-18 Variants

Methods of preparing and administering IL-18 variants of the disclosure as well as the nucleic acids described herein, the vectors described herein, the host cell cells described herein or the compositions described herein to a subject are well known to or are readily determined by those skilled in the art. The route of administration of the antigen binding proteins of the current disclosure may e.g., be oral, parenteral, by inhalation, or topical. The term parenteral as used herein includes intravenous, intraarterial, intraperitoneal, intramuscular, subcutaneous, rectal or vaginal administration. In certain embodiments, the antigen binding proteins or fusion proteins are administered intravenously. The term intraocular as used herein includes, but is not limited to, subconjunctival, intravitreal, retrobulbar, or intracameral. The term topical as used herein includes, but is not limited to, administration with liquid or solution eye drops, emulsions (e.g., oil-in-water emulsions), suspensions, and ointments.

While all these forms of administration are clearly contemplated as being within the scope of the current disclosure, a form for administration would be a solution for injection. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

Effective doses of the compositions of the present disclosure, for the treatment of the related conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

As previously discussed, the antigen binding proteins or fusion proteins of the present disclosure, conjugates or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the antigen binding proteins shall be held to mean an amount sufficient to achieve effective binding to an antigen and to achieve a benefit, e.g., to ameliorate symptoms of a disease or disorder or to detect a substance or a cell. In the case of tumor cells, the antigen binding proteins will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antigen binding proteins of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of antigen binding proteins described in the current disclosure may prove to be particularly effective. Similarly, the nucleic acids described herein, the vectors described herein, the host cell cells described herein (in particular the immune cells bearing a CAR) or the compositions described herein may be administered to a human or other animal in accordance with the methods of treatment described above in an amount sufficient to produce a therapeutic or prophylactic effect.

“Efficacy” or “in vivo efficacy” as used herein refers to the response to a therapy by the pharmaceutical composition of the disclosure, using e.g., standardized response criteria, such as standard ophthalmological response criteria. The success or in vivo efficacy of the therapy using a pharmaceutical composition of the disclosure refers to the effectiveness of the composition for its intended purpose, i.e., the ability of the composition to cause its desired effect. The in vivo efficacy may be monitored by established standard methods for the specific diseases. In addition, various disease specific clinical chemistry parameters and other established standard methods may be used.

In some embodiments, the compounds and cells described herein are administered in combination with one or more different pharmaceutical compounds. Generally, therapeutic use of the compounds and cells described herein may be in combination with one or more therapies selected from the group of antibody therapy, chemotherapy, cytokine therapy, dendritic cell therapy, gene therapy, hormone therapy, laser light therapy, radiation therapy or vaccine therapy.

### Methods of Treatment

Methods of treating diseases or disorders that would benefit from an IL-18 variant therapy are described herein. A patient afflicted with said disease or disorder can be administered the IL-18 variants of the disclosure. In certain embodiments, the disease or disorder is cancer. In certain embodiments, the disease or disorder is a viral infection.

In an aspect, the disclosure provides a method of treating cancer in a patient in need thereof comprising administering to the patient a therapeutically effective amount of the pharmaceutical compositions or IL-18 variants described herein.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Generation of Interleukin 18 Variants

Two methods were employed to generate IL-18 variants that possess reduced binding to IL-18 binding protein (IL-18BP) with maintained or improved binding to IL-18Rα: 1) a rational design approach, and 2) a yeast display approach.

### Rational Design

IL-18 Rational Design variants were generated through computational modeling and simulation using either CCG-MOE, Chimera, Schroedinger, or PyMol software to identify interactions between IL-18 and receptors or BP. Variants were designed to either enhance or reduce contact formation as desired. Starting structures for modeling include PDB: 3WO4, 3F62, 7AL7. In some instances, rational design was conducted by Cyrus using Rosetta based design.

### Yeast Display

IL-18 yeast display library generation, screening, and sequencing were done by Curia (formerly LakePharma). Libraries were designed based on residues showing propensity to influence I8 receptor or BP affinity as determined from rational design and computational modeling approach. Libraries were screened and sorted for receptor or IL18BP binding as desired, and top clones were sequenced.

Based on these two approaches, a series of IL-18 variants were generated and tested for desired activity.

### General Materials and Methods:

Gene Synthesis and Subcloning into a Mammalian Expression Vector

Synthesis and subcloning of the IL-18 variant genes for expression of the designed constructs was carried out using standard molecular biology methods.

### Protein Expression

All proteins were expressed using the EXPI293™ Expression System Kit (ThermoFisher Scientific) following the manufacturer's protocol.

### Protein Purification

After harvesting the expression culture supernatant, expressed protein was captured depending on the nature of the fusion protein. Proteins containing IgG Fc (mouse or human) were captured on a protein A column, and the column was washed with up to 5 column volume of PBS. The protein was eluted from the column by lowering the pH of the running buffer and directly neutralized with Tris buffer pH 8. The purified protein was then dialyzed overnight against PBS or further purified using size exclusion chromatography on either superdex 200 or superdex 75 column and AKTA Pure FPLC system. For proteins containing a hexahistidine (6×His) fusion, protein in the supernatant was captured on Ni-NTA sepharose resin and eluted with increasing concentrations of imidazole. The purified protein was then dialyzed overnight against PBS or further purified using size exclusion chromatography on either superdex 200 or superdex 75 column and AKTA Pure FPLC system.

### Octet Biolayer Interferometry for Affinity Determination

Octet 96red instrument was used to determine affinities of IL18 proteins to BP or receptors. 5-10 ug/ml analyte proteins were used for loading on Sartorius tips (AHC or SA). Ligands were diluted 2 to 3-fold each step for serial dilutions from 10 uM, 100 nM, or 50 nM depending on the affinity. 120-180 s loading, 200 s association and 800 s dissociation were used for kinetics. 3-7 data points were used to calculate affinities.

### HekBlue Cell Line

HEK-Blue IL-18 reporter cells (InvivoGen) express IL-18 receptors with a functional NF-kb-AP1 signaling pathway fused to the secreted embryonic alkaline phosphatase (SEAP) gene. Treatment of bioactive IL-18 on cells activates the signaling pathway and induces secretion of SEAP into cell supernatant which can be detected by a colorimetric substrate (Quanti-Blue).

HEK-Blue IL-18 reporter cells were maintained in complete media (DMEM, 4.5 g/l glucose, 2 mM L-Glutamine, 10% (v/v) heat-inactivated fetal bovine serum, 100 U/ml penicillin, 100 μg/ml streptomycin, 100 μg/ml Normocin, 15 ug/ml Blasticidin, 50 ug/ml Zeocin, 100 ug/ml Hygromycin B.

### HEK-Blue IL-18 Activity & IL-18BP Blockade Assays

For activity and IL-18BP blockade measurements and screening, IL-18 variants were prepared in a dose-dependent titration and co-incubated with or without a fixed concentration of IL-18BP for 30 minutes at room temperature. Cells were detached and resuspended for 12,500 cells/well for a final volume with IL-18 mixture of 50 μl/well in a 384-well tissue-culture treated plate, incubated for 16-24 hours overnight at 37 C/5% CO2. 5 μl of cell supernatants from each well were then added to 45 μl Quanti-Blue detection solution, incubated for 30-60 minutes at 37 C, read on a spectrophotometer at 620-655 nm wavelength.

### Human PBMC Stimulated IFN Gamma Assay

Constructs for testing were diluted in RPMI+10% FBS+1 ng/ml IL-12p70 (Peprotech) and added to 96 well round bottom plates+/−IL-18BP. Healthy donor PBMCs were thawed from frozen and plated at 100,000 cells/well. Plates were incubated at 37 C/5% CO2 for 24 hours. Supernatants were collected and Interferon gamma measured using Human IFNg Quantikine ELISA (R&D Systems).

### Mouse Splenocyte Stimulated IFN Gamma Assay

Freshly harvested C57/B16 mouse spleens were dissociated using Miltenyi Gentle MACs C-tubes. Red blood cells were lysed (ACK ThermoFisher) and cells washed with sterile PBS. Cells were plated in T-75 flasks for one hour in RPMI+10% FBS. Non-adherent cells were removed and re-plated in T-75 flasks coated with anti-CD3e (R&D Systems) in the presence of anti-CD28 (R&D systems). Flasks were incubated 72 hours at 37 C/5% CO2. Cells are removed from flasks, centrifuged to pellet and resuspended in RPMI+10% FBS+2 ng/ml IL-12p70 (R&D Systems). Cells were incubated 24 hours, and cells were then harvested by resuspending in media containing 0.1 ng/ml IL-12p70. Cells were plated at 40,000 cells/well in round bottom 96 well plates in the presence of IL-18 constructs+/−IL-18BP. Cells were incubated 24 hours. Supernatants were collected and Interferon gamma measured using Mouse IFNg Quantikine ELISA (R&D Systems).

### Human In Vitro CD8 T Cell Exhaustion Protocol

Human CD8+ T cells were enriched from frozen PBMCs using the CD8+ T cell Miltenyi isolation kit according to the manufacturer's instructions. Purified CD8 T cells were plated at a 1×10⁶ cells per milliliter in 10% FBS in RPMI medium. Cells were stimulated with T-activator CD3/CD28 Dynabeads (Life Technologies) following manufacturer's recommendations in the presence of 25 U/mL rhIL-2. Every 48 hours, cells were counted, washed, and re-stimulated with a fresh batch of Dynabeads and low dose rhIL-2. After four stimulations, the exhausted CD8 T cells were washed and re-stimulated with Dynabeads and rhIL-2 with or without Alkermes immune-therapies for four to five days. Supernatants were collected and IFNγ was measured using the R and D Systems ELISA kit.

### Human PBMC/A549 Killing Assay Protocol

A549 cells transfected with Nuclight Green (Sartorius) were plated in clear bottom plates and allowed to adhere overnight. Normal human PBMCs were thawed into RPMI-10% FBS-20 U/ml IL-2 and allowed to recover overnight.

PBMCs were plated over A549 cells at a 3:1 ratio in the presence of 1 ng/ml IL-12p40. IL-18 molecules were added at the time of plating. Cells were incubated for 5 days and imaged by IncuCyte every 4 hours. Number of A549 cells were calculated for each well and used to determine efficacy of killing.

### Mouse Reporter Assay—Cell Line

PathHunter U2OS mIL18-NF-kb reporter cell (Eurofin DiscoverX custom cells) express mouse IL-18 receptors (mouse IL18R1 NP_032391.1 & mouse IL18RAP NP_034683.1) with a NFkb signaling reporter gene that expresses ePL-tagged protein (ePL is a fragment of the b-galactosidase enzyme). Mouse IL18-mediated pathway activation leads to an increase in expression of the tagged reporter protein, which can be quantified by addition of complementing β-gal enzyme acceptor to the detection reagent in a homogeneous assay format.

Cells were maintained in manufacturer's cell culture reagents (DiscoverX Cat. No. 92-3103G) supplemented with 0.25 ug/ml puromycin, 250 ug/ml hygromycin B, 500 ug/ml G418, 100 U/ml penicillin, 100 ug/ml streptomycin. Cells are passaged every 2-3 days at 1:3 or 1:6, dissociated using cell detachment reagent (DiscoverX Cat. No. 92-0009) and maintained at 37 C, 5% CO2.

### Mouse Reporter Assay—IL-18 Activity & IL-18BP Blockade Assays

Cells were detached and resuspended in cell plating reagent (DiscoverX AssayComplete Cell Plating 3 Reagent Cat. No. 93-0563R3A) for 2,000 cells/well in 20 ul/well into 384-well white clear bottom tissue culture plate for 24 hours at 37 C/5% CO2.

For activity and IL-18BP blockade measurements and screening, IL-18 variants were prepared in a dose-dependent titration and co-incubated with or without a fixed concentration of IL-18BP for 30 minutes at room temperature. 5 ul of 5×IL-18 mixture was added to cell plate and incubated for 6 hours at 37 C/5% CO2. Detection reagents (DiscoverX PathHunter ProLabel/ProLink Detection Kit Cat. No. 93-0812) were prepared according to manufacturer's protocol and 25 ul detection mixture added to cell plate and incubated in the dark for 1 hour at room temperature. Plate was read on a luminescence plate reader.

### Gene Synthesis, Protein Expression, and Purification for IL-18 Non-Fusion Constructs (Naked)

All 6his-tagged non-fusion IL-18 proteins (naked) were gene synthesized, subcloned, expressed, and purified by WuxiBiologics with an N-terminal SUMO-tag that was cleaved during purification.

### Example 2—Characterization of Interleukin 18 Variants

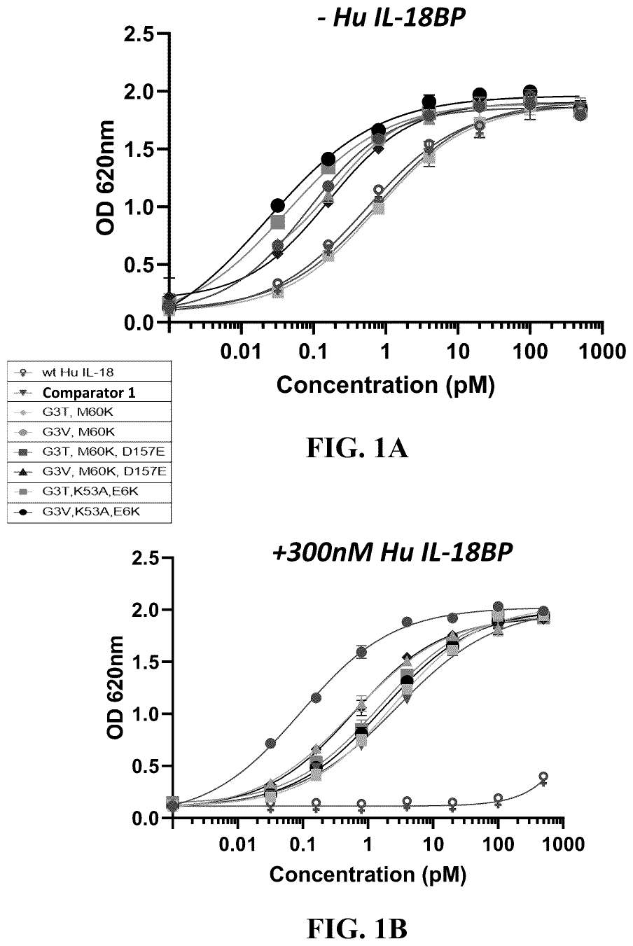

Several IL-18 variants were tested in a HEK Blue IL-18 activity reporter assay at a range of concentrations, both in the absence of human IL-18BP and in the presence of 300 nM human IL-18BP. As shown in FIG. 1A and FIG. 1B, all IL-18 variants retained activity in the presence of IL-18BP, while WT IL-18 activity was suppressed.

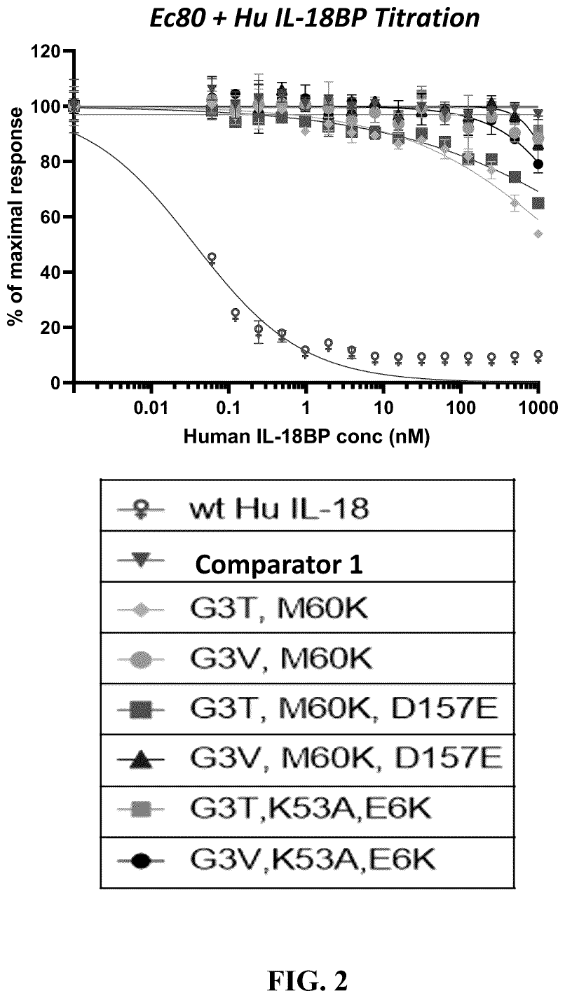

The relative activity of select IL-18 variants in the presence of titrated human IL-18BP was also tested. As shown in FIG. 2, all IL-18 variants retained activity in the presence of increasing levels of IL-18BP, while WT IL-18 activity was quickly suppressed.

TABLE 5
EC50 values for select IL-18 variants and binding affinity (KD in
nM) to IL-18BP, an IL-18R alpha/beta pair (IL-18Rab), IL-18R alpha.

| | HEK-Blue Activity Assay | | Octet Binding | | |
|---|---|---|---|---|---|
| | Ec50 (pM) | | KD Affinity (nM) | | |
| | −hu | +300 nM hu | hu IL- | hu IL- | hu IL- |
| Variant | IL-18BP | IL-18BP | 18BP | 18Rab | 18Ra |
| WT human IL-18 | 0.53 | — | 0.3 | 0.12 | 64 |
| Comparator 1 | 0.08 | 0.09 | NBD | 0.43 | 16 |
| G3T, M60K | 0.76 | 2.40 | 779 | 0.02 | 555 |
| G3V, M60K | 0.18 | 0.59 | 91 | 0.07 | 41 |
| G3T, M60K, D157E | 0.75 | 2.77 | 686 | 0.10 | 693 |
| G3V, M60K, D157E | 0.16 | 0.60 | 87 | 0.13 | 43 |
| G3T, E6K, K53A | 0.04 | 1.49 | 6 | 0.32 | 10 |
| G3V, E6K, K53A | 0.02 | 1.71 | 19 | 0.27 | 52 |

“NBD” corresponds to no binding detected.

All of the variants tested above had low EC50/high activity in the HEK-Blue assay in the presence of high 300 nM BP levels and tight binding to IL-18Rα.

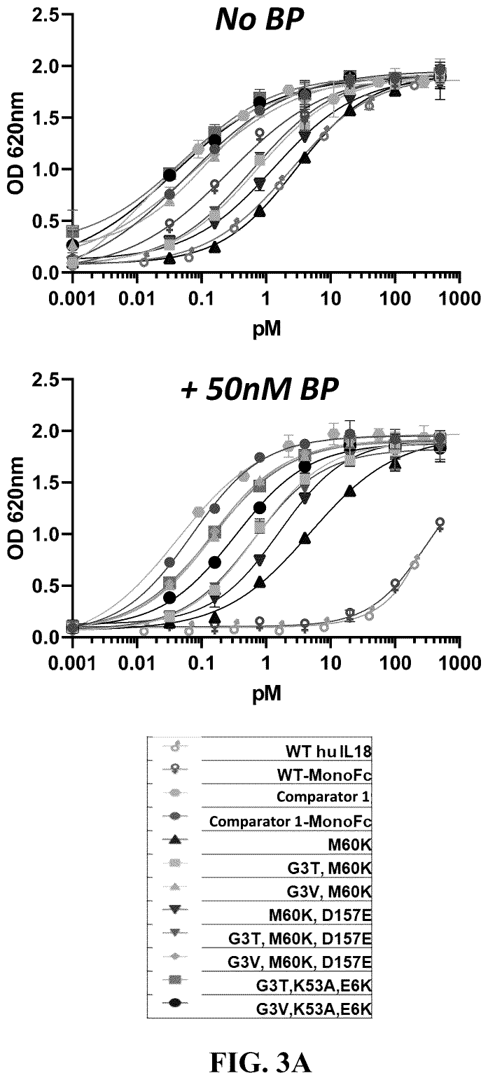

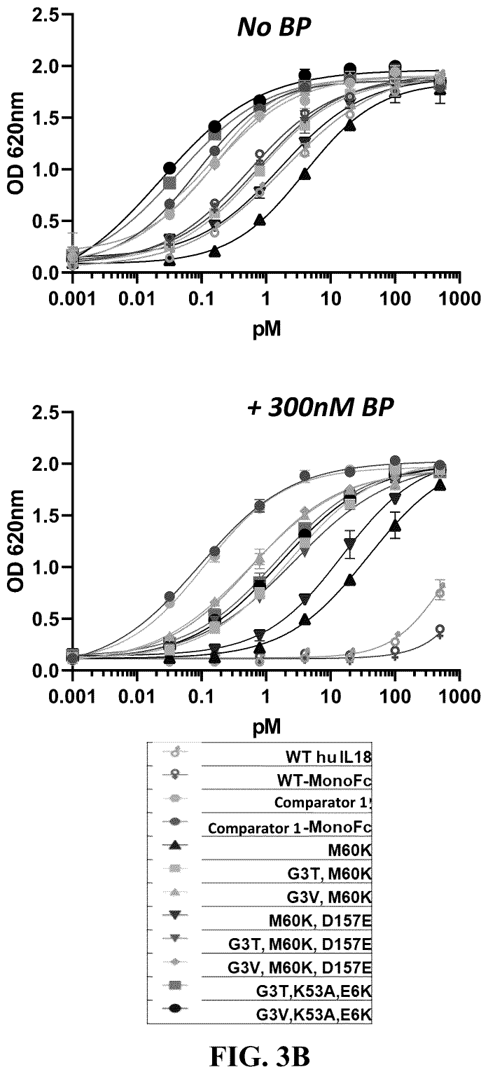

Several IL-18 variants were tested in the HEK Blue IL-18 activity reporter assay in the presence of 50 nM or 300 nM human IL-18BP. As shown in FIG. 3A and FIG. 3B, all IL-18 variants retained activity in the presence of IL-18BP, while WT IL-18 activity was suppressed. Table 6 below provides EC50 values for FIG. 3 with a fold change between IL-18BP vs. no IL-18BP.

TABLE 6
EC50 values for select IL-18 variants.

| | NO | +50 nM | Fold | NO | +300 nM | Fold |
|---|---|---|---|---|---|---|
| | BP | BP | Change | BP | BP | Change |
| | EC50 | EC50 | (BP/NO | EC50 | EC50 | (BP/NO |
| Variant | pM | pM | BP) | pM | pM | BP) |
| WT hu IL18 | 2.41 | — | — | 1.92 | — | — |
| WT-MonoFc | 0.26 | — | — | 0.53 | — | — |
| Comparator 1 | 0.02 | 0.04 | 2.50 | 0.13 | 0.11 | 0.83 |
| Comparator | 0.06 | 0.07 | 1.16 | 0.08 | 0.09 | 1.11 |
| 1-MonoFc | | | | | | |
| M60K | 2.90 | 4.77 | 1.64 | 3.96 | 38.29 | 9.67 |
| G3T, M60K | 0.71 | 0.70 | 0.99 | 0.76 | 2.40 | 3.14 |
| G3V, M60K | 0.12 | 0.16 | 1.25 | 0.18 | 0.59 | 3.25 |
| M60K, D157E | 1.30 | 1.62 | 1.25 | 1.67 | 15.12 | 9.04 |
| G3T, M60K, | 0.62 | 0.65 | 1.05 | 0.75 | 2.77 | 3.69 |
| D157E | | | | | | |
| G3V, M60K, | 0.08 | 0.14 | 1.74 | 0.16 | 0.60 | 3.74 |
| D157E | | | | | | |
| G3T, K53A, | 0.05 | 0.14 | 2.79 | 0.04 | 1.49 | 35.20 |
| E6K | | | | | | |
| G3V, K53A, | 0.04 | 0.33 | 8.67 | 0.02 | 1.71 | 74.76 |
| E6K | | | | | | |

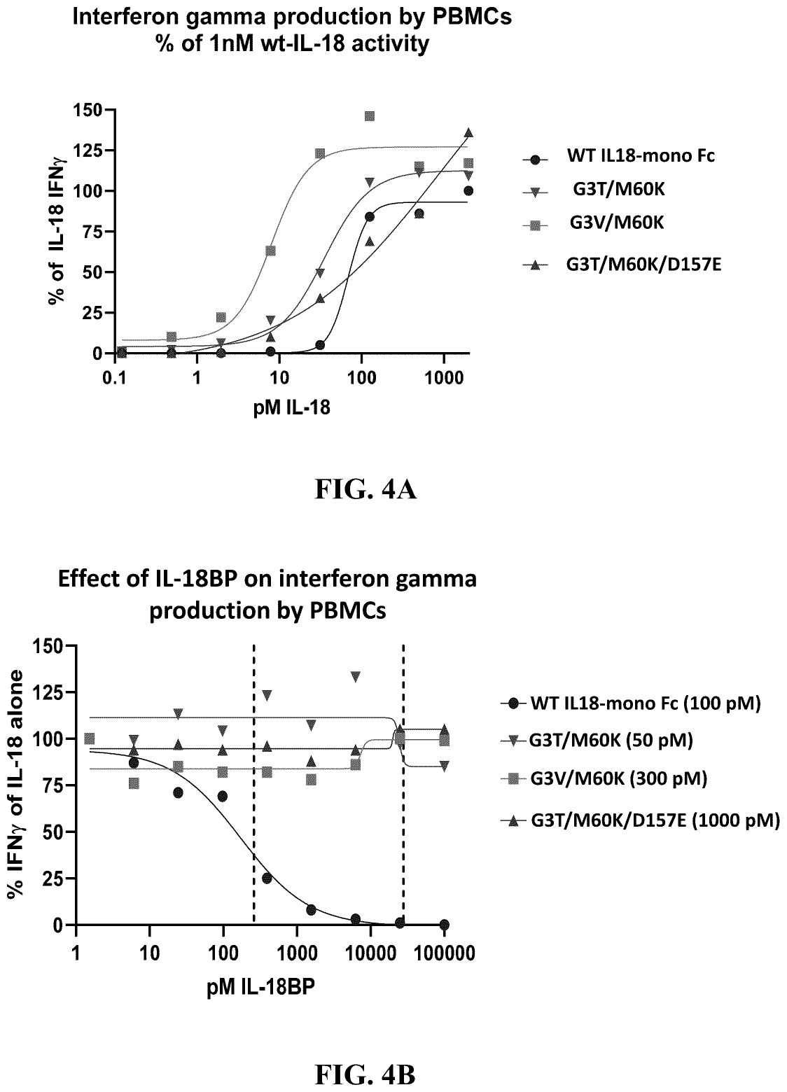

IL-18 variants with at least the M60K substitution were next tested for potency in a human PBMC stimulated IFN gamma assay. As shown in FIG. 4A and FIG. 4B, each of the variants stimulated IFN gamma expression at a level similar to or better than WT IL-18. This stimulation occurred even in the presence of IL-18BP. The EC50/IC50 values are reported below in Table 7.

TABLE 7
EC50/IC50 values for IFN gamma expression
in PBMCs incubated with IL-18 variants.

| | Potency - no IL-18BP | | Potency - with IL-18BP | |
|---|---|---|---|---|
| IL-18 Type | (EC50) | | (IC50) | |
| IL-18 wt-mono-Fc | 69 | pM | 96 | pM |
| G3T/M60K | 35 | pM | >100,000 | pM |
| G3V/M60K | 8 | pM | >100,000 | pM |
| G3T/M60K/D157E | 872 | pM | >100,000 | pM |

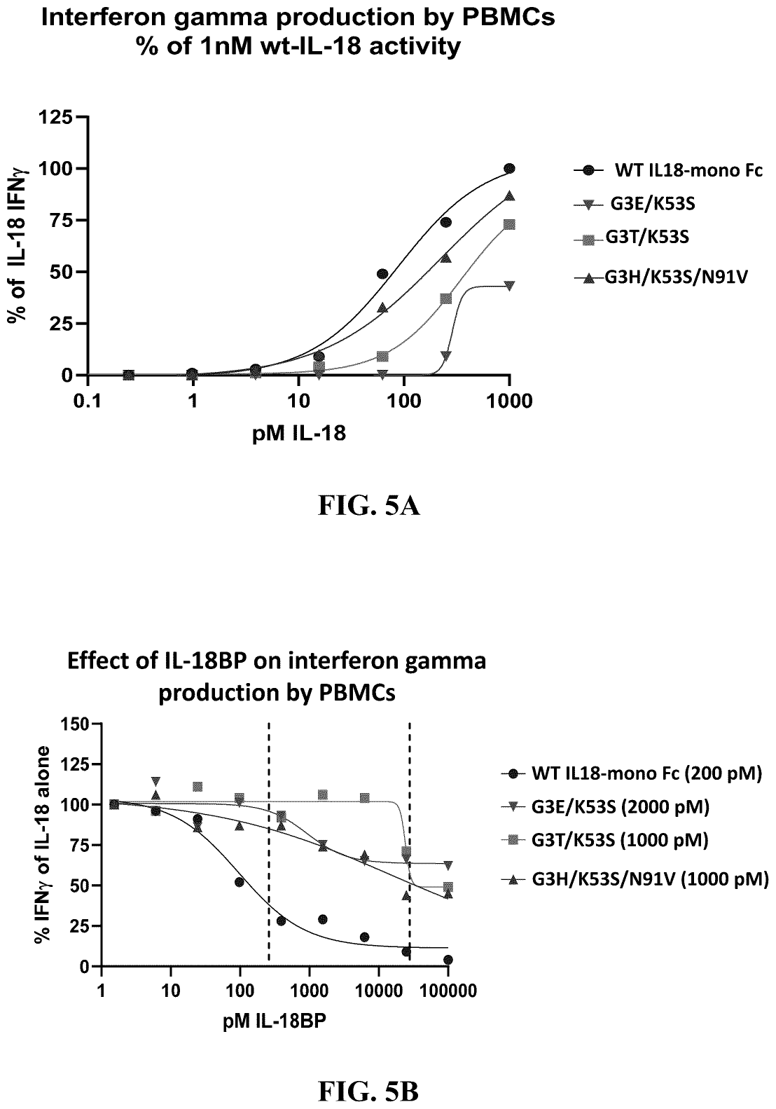

IL-18 variants with at least the K53S substitution were next tested for potency in a human PBMC stimulated IFN gamma assay. As shown in FIG. 5A and FIG. 5B, each of the variants stimulated IFN gamma expression at a level similar to or better than WT IL-18. This stimulation occurred even in the presence of IL-18BP. The EC50/IC50 values are reported below in Table 8.

TABLE 8
EC50/IC50 values for IFN gamma expression
in PBMCs incubated with IL-18 variants.

| | Potency - no IL-18BP | | Potency - with IL-18BP | |
|---|---|---|---|---|
| IL-18 type | (EC50) | | (IC50) | |
| IL-18 wt | 87 | pM | 96 | pM |
| G3E-K53S | *289 | pM | 925 | pM |
| G3T-K53S | *349 | pM | 15,304 | pM |
| G3H-N91V-K53S | *221 | pM | 24,267 | pM |

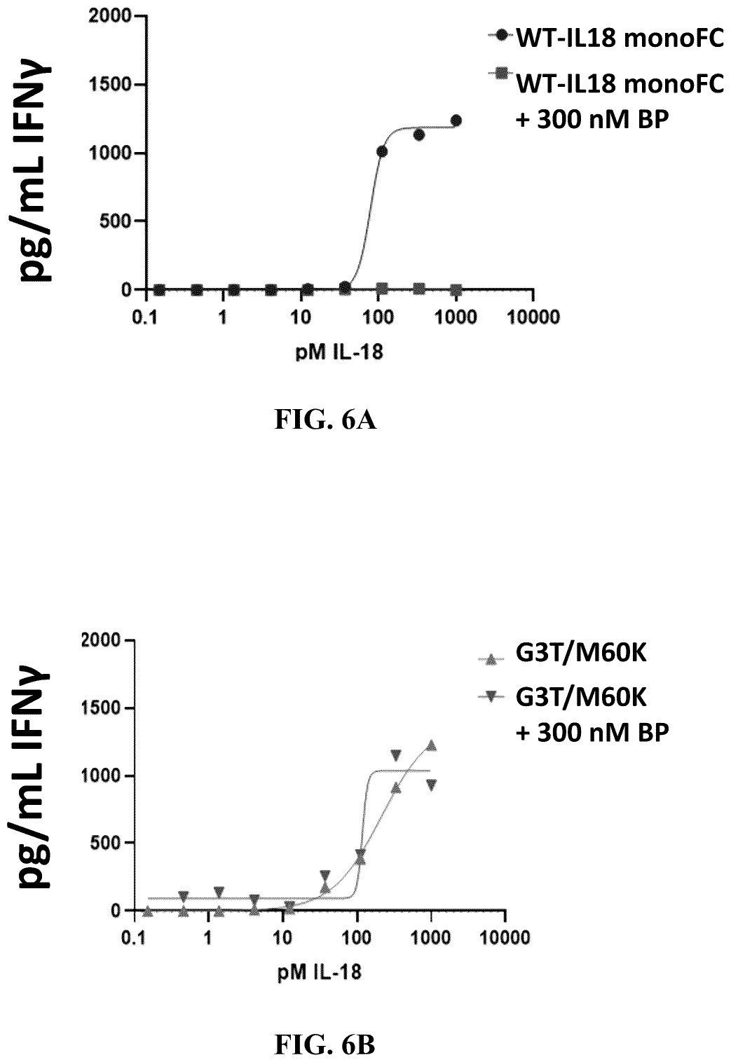

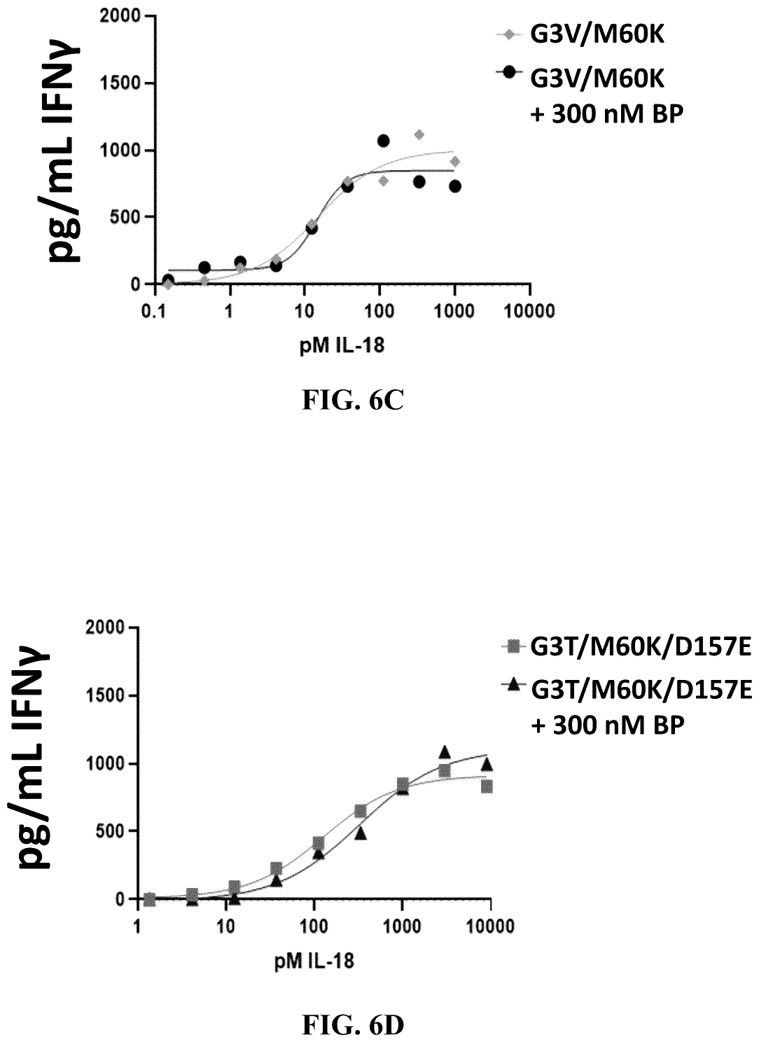

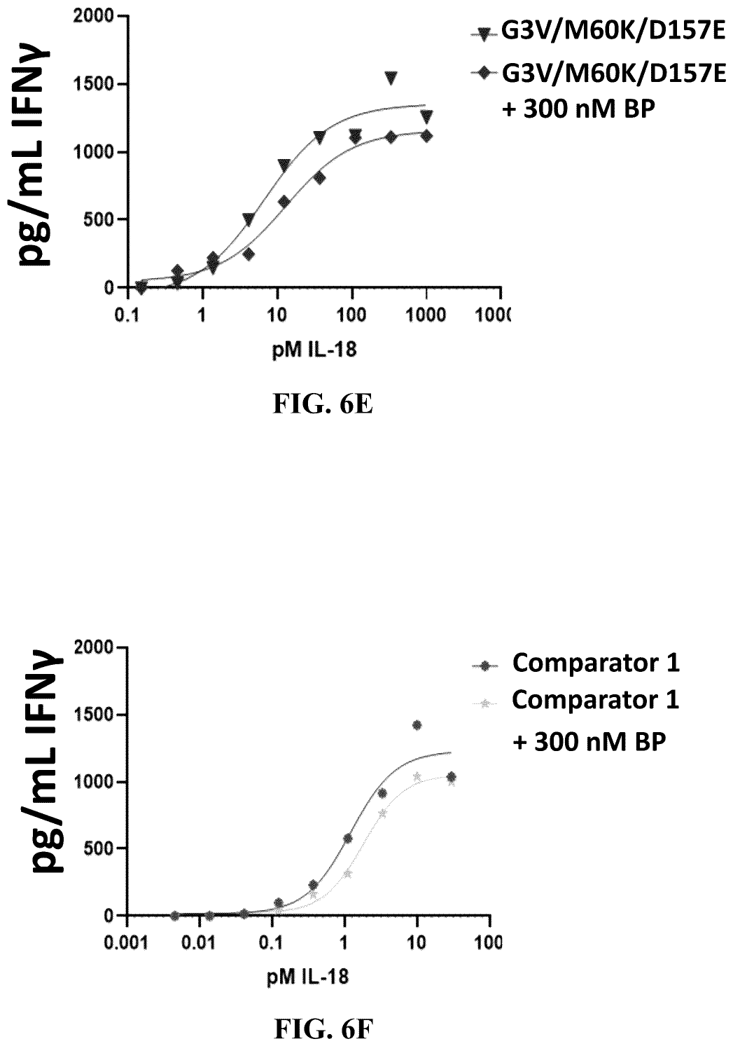

IL-18 variants with at least the M60K substitution were tested again for potency in a human PBMC stimulated IFN gamma assay with or without 300 nM IL-18BP. As shown in FIG. 6A-FIG. 6F, each of the variants stimulated IFN gamma expression at a level similar to or better than WT IL-18. This stimulation occurred even in the presence of IL-18BP. The EC50 values are reported below in Table 9.

TABLE 9
EC50 values for IFN gamma expression in
PBMCs incubated with IL-18 variants.

| IL-18 type | EC50 pM |
|---|---|
| WT-IL18 monoFC | 78.92 |
| WT-IL18 monoFC + 300 nM BP | >1000 |
| G3T/M60K | 214.3 |
| G3T/M60K + 300 nM BP | 117.4 |
| G3V/M60K | 14.91 |
| G3V/M60K + 300 nM BP | 14.37 |
| G3T/M60K/D157E | 126.6 |
| G3T/M60K/D157E + 300 nM BP | 323.1 |
| G3V/M60K/D157E | 6.609 |
| G3V/M60K/D157E + 300 nM BP | 13.01 |
| Comparator 1 | 1.215 |
| Comparator 1 + 300 nM BP | 1.824 |

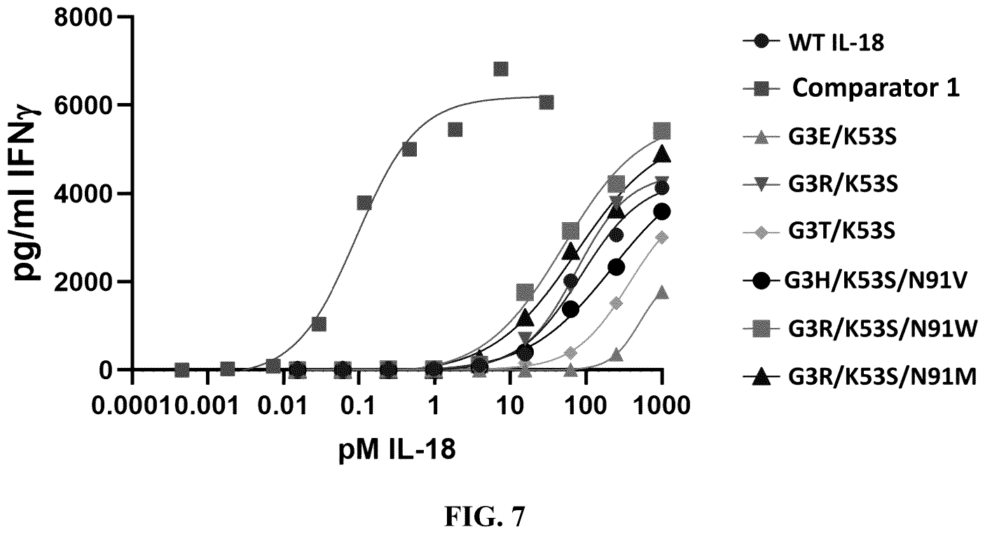

Additional IL-18 variants with the K53S or K53A substitution were tested again for potency in a human PBMC stimulated IFN gamma assay. As shown in FIG. 7, each of the variants stimulated IFN gamma expression at a level similar to or better than WT IL-18. The EC50 values are reported below in Table 10.

TABLE 10
EC50 values for IFN gamma expression in
PBMCs incubated with IL-18 variants.

| IL-18 type | EC50 pM |
|---|---|
| WT-IL18 | 86.22 |
| Comparator 1 | 0.093 |
| G3E/K53S | 516.7 |
| G3R/K53S | 73.83 |
| G3T/K53S | 367.8 |
| G3H/K53S/N91V | 221.5 |
| G3R/K53A/N91W | 49.84 |
| G3R/K53S/N91M | 73.92 |

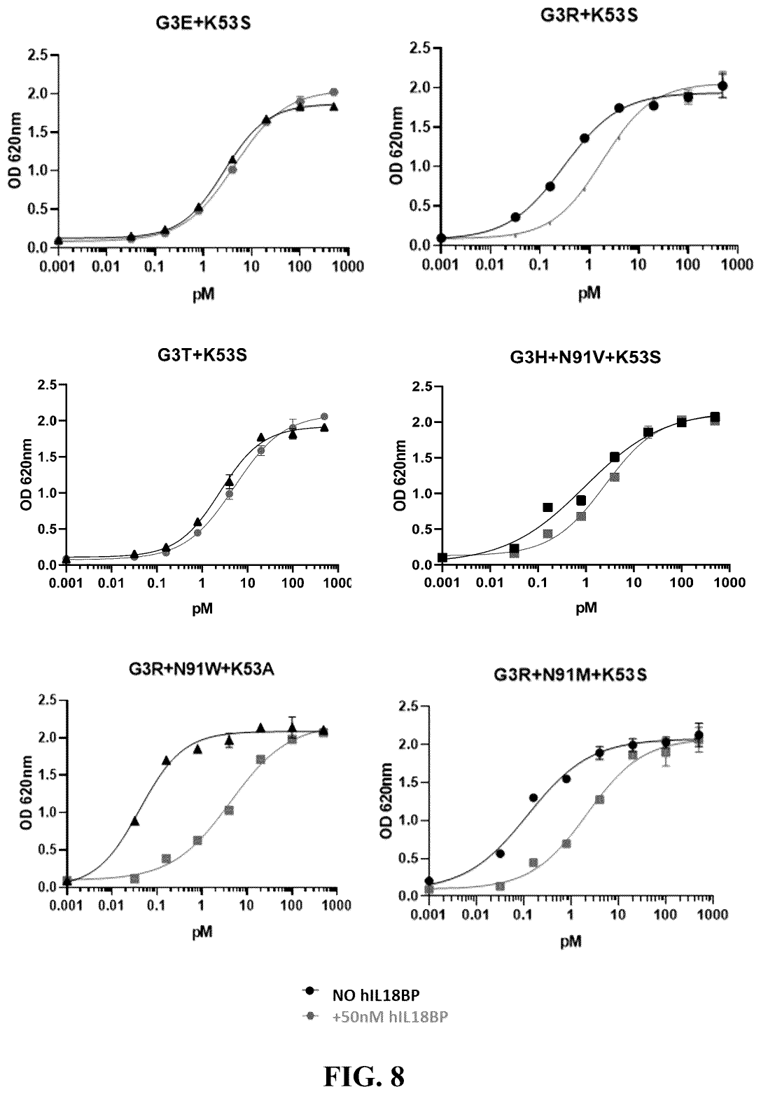

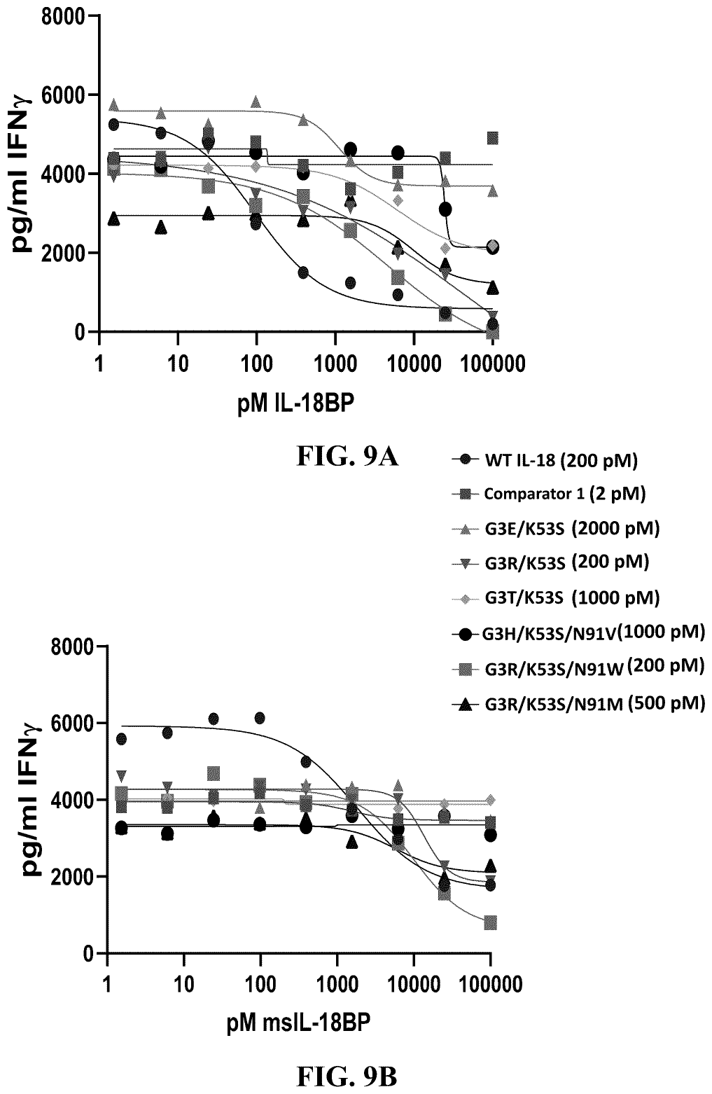

The TL-18 variants of FIG. 7 were tested in the HEK Blue IL-18 activity reporter assay in the presence of 50 nM human IL-18BP. As shown in FIG. 8, all IL-18 variants retained activity in the presence of IL-18BP. Moreover, all IL-18 variants retained activity in the presence of increasing levels of IL-18BP in the human PBMC stimulated IFN gamma assay. This was the case with titrations of human IL-18BP (FIG. 9A) and mouse IL-18BP (FIG. 9B).

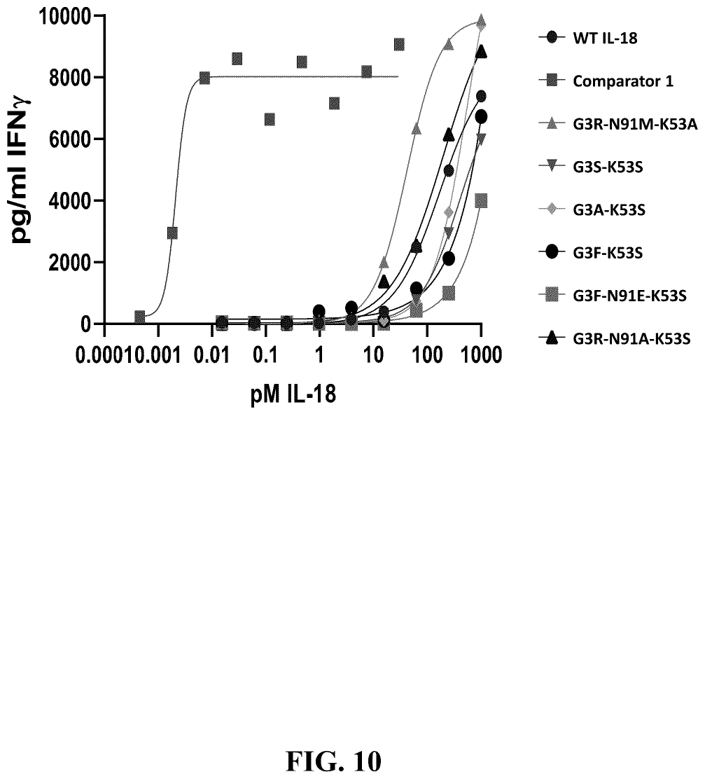

Yet more IL-18 variants with the K53S or K53A substitution were tested for potency in the human PBMC stimulated IFN gamma assay, combining K53S or K53A with different G3 and N91 mutations. As shown in FIG. 10, each of the variants stimulated IFN gamma expression at a level similar to WT IL-18. The EC50 values are reported below in Table 11.

TABLE 11
EC50 values for IFN gamma expression in
PBMCs incubated with IL-18 variants.

| IL-18 type | EC50 pM |
|---|---|
| WT-IL18 | 169.9 |
| Comparator 1 | 0.002 |
| G3R/K53A/N91M | 41.85 |
| G3S/K53S | 397.6 |
| G3A/K53S | 535.7 |
| G3F/K53S | >1000 |
| G3F/K53S/N91E | >1000 |
| G3R/K53S/N91A | 198.5 |

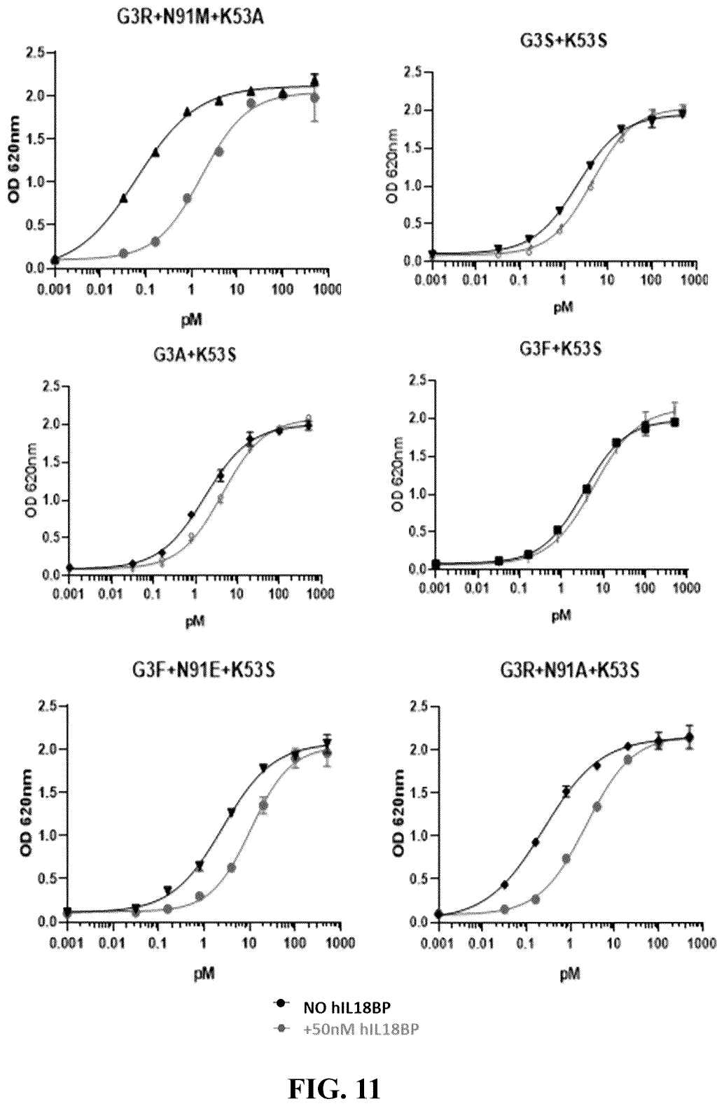

The IL-18 variants of FIG. 10 were tested in the HEK Blue IL-18 activity reporter assay in the presence of 50 nM human IL-18BP. As shown in FIG. 11, all IL-18 variants retained activity in the presence of IL-18BP.

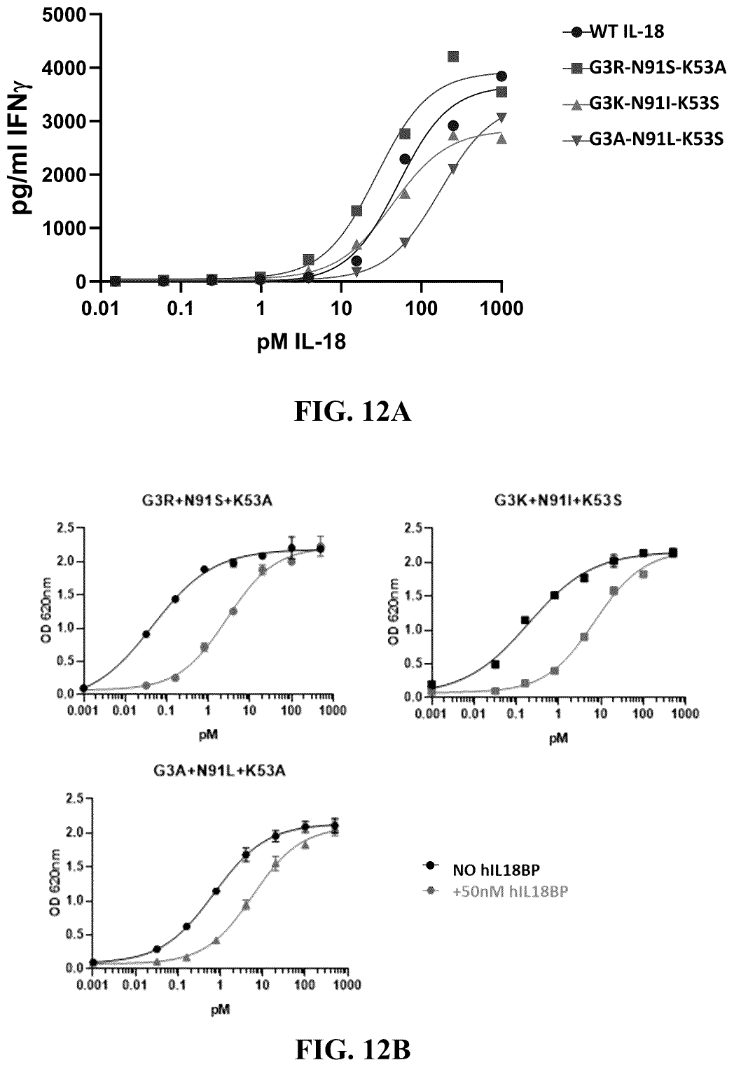

Three additional IL-18 variants with the K53S or K53A substitution were tested for potency in the human PBMC stimulated IFN gamma assay. As shown in FIG. 12A, each of the variants stimulated IFN gamma expression at a level similar to WT IL-18. The EC50 values are reported below in Table 12.

TABLE 12
EC50 values for IFN gamma expression in
PBMCs incubated with IL-18 variants.

| IL-18 type | EC50 pM |
|---|---|
| WT-IL18 | 51.85 |
| G3R-N91S-K53A | 27.66 |
| G3K-N91I-K53S | 43.47 |
| G3A-N91L-K53S | 171.2 |

The IL-18 variants of FIG. 12A were tested in the HEK Blue IL-18 activity reporter assay in the presence of 50 nM human IL-18BP. As shown in FIG. 12B, all IL-18 variants retained activity in the presence of IL-18BP.

IL-18 Variants with Exhausted T Cells

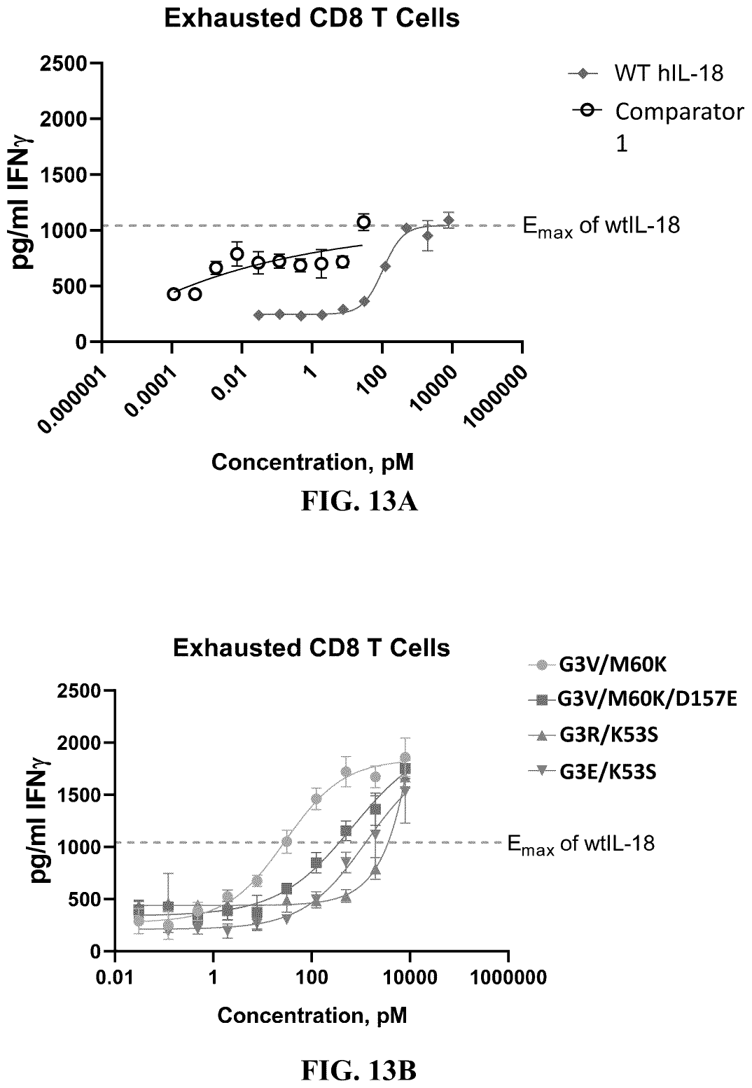

Select IL-18 variants were tested for their ability to stimulate IFN gamma expression in exhausted CD8+ T cells. The exhausted T cells were generated following the protocol described in Example 1 using multiple stimulations with CD3/CD28. Exhausted T cells display reduced effector function and worse IFN gamma expression. As shown in FIG. 13A, WT IL-18 and the IL-18 variant Comparator 1 poorly stimulated exhausted T cells, as shown by the low IFN gamma expression.

However, all tested IL-18 variants were capable of potently stimulating IFN gamma expression in exhausted T cells, suggesting an ability to restore T cell function (FIG. 13B). The G3V/M60K containing variants were particularly efficacious. The EC50 values are reported below in Table 13 and Table 14.

TABLE 13
EC50 values for IFN gamma expression in exhausted
T cells incubated with IL-18 variants.

| IL-18 type | EC50 pM |
|---|---|
| WT-IL18 | 105.9 |
| Comparator 1 | 2.6e−26 |
| G3V/M60K | 29.2 |
| G3T/M60K/D157E | 685.3 |
| G3R/K53S | 1310 |
| G3E/K53S | >10,000 |

TABLE 14
EC50 values for HEK-blue assay activity and
KD binding affinity with IL-18 variants.

| | HEK-Blue Activity Assay | | Octet Binding | | |
|---|---|---|---|---|---|
| | Ec50 (pM) | | KD Affinity (nM) | | |
| | −hu | +50 nM hu | hu IL- | hu IL- | hu IL- |
| Variant | IL-18BP | IL-18BP | 18BP | 18Rab | 18Ra |
| G3V/M60K | 0.12 | 0.16 | 91 | 0.07 | 41 |
| G3T/M60K/D157E | 0.62 | 0.65 | 686 | 0.10 | 693 |
| G3R/K53S | 0.31 | 1.84 | 4.03 | 0.09 | 197 |
| G3E/K53S | 2.69 | 4.41 | 396 | 0.31 | 8920 |

### G3/K53 or G3/K53/N91 IL-18 Variants—Activity and IL-18BP Binding Affinity

Select G3/K53-substitution containing IL-18 variants and select G3/K53/N91-substitution containing TL-18 variants were tested for activity in the HEK-blue activity assay and for binding affinity to IL-18BP. As shown in the data of Table 15 and Table 16 below, each variant had similar or better activity than WT IL-18, and retained substantial activity in the presence of IL-18BP. Moreover, each variant had a faster Koff rate (k_{dis}(1/s)) than WT IL-18BP.

TABLE 15
EC50 values for HEK-blue assay activity and KD binding affinity
with G3/K53-substitution containing IL-18 variants.

| | HEK-Blue | | | | |
|---|---|---|---|---|---|
| | Ec50 (pM) | | Octet binding to IL18BP | | |
| | | +IL-18BP | KD | | |
| Variant | −IL-18BP | (50 nM) | (nM) | kₒₙ(1/Ms) | K_{dis}(1/s) |
| Comparator 1 | 0.15 | 0.22 | — | — | — |
| WT IL-18 | 1.09 | >200 | 0.5 | 1.79E+05 | 9.28E−05 |
| G3E-K53S | 2.69 | 4.41 | 396.0 | 7.85E+04 | 3.11E−02 |
| G3S-K53S | 2.06 | 4.43 | 12.0 | 2.83E+05 | 3.38E−03 |
| G3A-K53S | 1.69 | 4.45 | 16.2 | 2.45E+05 | 3.98E−03 |
| G3R-K53S | 0.31 | 1.84 | 4.0 | 3.56E+05 | 1.43E−03 |
| G3F-K53S | 3.39 | 5.88 | 11.8 | 4.08E+05 | 4.80E−03 |
| G3T-K53S | 2.50 | 5.18 | 18.0 | 2.63E+05 | 4.74E−03 |

TABLE 16
EC50 values for HEK-blue assay activity and KD binding affinity
with G3/K53/N91-substitution containing IL-18 variants.

| | HEK-Blue | | | | |
|---|---|---|---|---|---|
| | Ec50 (pM) | | Octet binding to IL18BP | | |
| | | +IL-18BP | KD | | |
| Variant | −IL-18BP | (50 nM) | (nM) | kₒₙ(1/Ms) | k_{dis}(1/s) |
| Comparator 1-MonoFc | 0.16 | 0.16 | — | — | — |
| WT IL-18-MonoFc | 1.60 | >200 | 0.5 | 1.79E+05 | 9.28E−05 |
| G3R-N91M-K53A | 0.06 | 1.67 | 1.5 | 2.08E+05 | 3.10E−04 |
| G3R-N91M-K53S | 0.12 | 2.16 | 3.3 | 2.23E+05 | 7.35E−04 |
| G3R-N91A-K53S | 0.25 | 2.27 | 3.4 | 2.13E+05 | 7.28E−04 |
| G3R-N91S-K53A | 0.05 | 2.80 | 0.9 | 1.91E+05 | 1.62E−04 |
| G3R-N91W-K53A | 0.04 | 4.22 | 1.0 | 1.85E+05 | 1.85E−04 |
| G3F-N91E-K53S | 2.55 | 10.66 | 9.1 | 2.13E+05 | 1.95E−03 |
| G3A-N91L-K53S | 0.71 | 5.82 | 4.8 | 2.16E+05 | 1.03E−03 |
| G3H-N91V-K53S | 0.94 | 2.51 | 8.6 | 3.59E+05 | 3.10E−03 |
| G3K-N91I-K53S | 0.20 | 7.05 | 1.9 | 2.18E+05 | 4.06E−04 |

### Mouse IL-18 Variants

Mouse IL-18 and variants thereof were tested in IFN gamma assays to identify useful variants that may act as surrogates of the human IL-18 variants described herein. The mouse versions of the human IL-18 variants can then be used in vivo. Mouse splenocytes were used to measure IFN gamma upon incubation with WT mouse IL-18, comparator mouse IL-18 variants (Comparator 4 and Comparator 5), and IL-18 variants that act as surrogates of the human IL-18 variants. Table 17 below recites the EC50 values for the WT IL-18 and comparator mL-18 variants in the IFN gamma assay. Table 18 below recites the EC5 values for the mouse IL-18 variant surrogates in the IFN gamma assay.

TABLE 17
EC50 values of mouse WT IL-18 and mouse IL-18
comparator variants for IFN gamma expression.

| Mouse IL-18 variants | EC50 (pM) |
|---|---|
| Comparator 5-6his (6xHis tag) | 1.682 |
| Comparator 4-6his | 3.699 |
| Comparator 5 | 38.86 |
| Comparator 4 | 25.4 |
| (Comparator 5)-Fc | 3.593 |
| (Comparator 4)-Fc | 271.2 |
| wild type | 13.98 |

TABLE 18
EC50 values of mouse IL-18 variant surrogates
for IFN gamma expression.

| Mouse IL-18-monoFc variants | EC50 (pM) |
|---|---|
| G3T, H6K, K52A | ~52.30 |
| G3V, H6K, K52A | ~161.3 |
| G3T, K52A | ~6.004e−005 |
| G3V, K52A | ~0.03756 |
| wild type | 13.98 |

Incubating mouse ML-18 variants in a mouse IL-18 reporter assays with or without mouse IL-18BP demonstrated that the assay was able to discern IL-18BP sensitivity. Table 19 below recites the EC50 values for the WT IL-18, comparator IL-18 variants, and IL-18 variant surrogates in the reporter assay.

TABLE 19
EC50 values of mouse IL-18 variant surrogates for activity.

| | Mouse IL-18BP | +Mouse IL-18BP |
|---|---|---|
| IL-18 type | EC50 (pM) | 50 Nm ec50 (pM) |
| WT human IL-18 | 1.47 | >500 pM |
| WT mouse IL-18 | 0.61 | >500 pM |
| Comparator 5 | 1.49 | 0.91 |
| G3T, H6K, K52A | 1.76 | 1.69 |
| G3V, H6K, K52A | 0.90 | 4.86 |
| G3T, K52A | 1.24 | 20.77 |
| G3V, K52A | 0.70 | 36.35 |

Several mouse variants were next tested for binding affinity to mouse IL-18R alpha and mouse IL-18BP. As shown in Table 20 below, all variants bound IL18R alpha with similar KD values as WT IL-18 and all variants had little to no binding to IL-18BP.

TABLE 20
Octet-measured affinity values of select mouse IL-18 variant.

| Mutation (on | Mouse IL18Ra | Mouse IL18BP |
|---|---|---|
| monoFc Scaffold) | KD (nM) | KD (nM) |
| Comparator 5 | 1.27 | NBD |
| WT | 13.3 | 2.24 |
| G3T/L59K | 12.3 | NBD |
| G3V/L59K | 14.5 | NBD |
| G3T/H6K/L59K | 12.8 | NBD |
| G3V/H6K/L59K | 24.2 | NBD |
| G3V/L59M | 17.4 | 2.47 |
| G3V/L59R | 51.5 | NBD |
| G3T, H6K, K52A | 42.7 | 78 nM |
| G3V, H6K, K52A | 4.85 | 14 nM |
| G3T, K52A | 97 | 11 nM |
| G3V, K52A | 8.85 | 2 nM |

### IL-18 Variant Conjugates

IL-18 is difficult to express in mammalian cells due to aggregation and often must be expressed in E. coli, which is undesirable for a future therapeutic to be administered to a patient. To improve IL-18 variant expression, the IL-18 variants described herein were linked to one of several different polypeptides to enhance expression. The polypeptides tested included a mono-Fc (a single CH2-CH3 Fc domain), a knob-in-hole (KiH)-Fc (two CH2-CH3 Fc domains that have heterodimerized though KiH mutations, with only one Fc domain being linked to the IL-18 variant), an Fc domain (two CH2-CH3 Fc domain that have dimerized, with both domains being linked to an IL-18 variant), and either human serum albumin (HSA) or mouse serum albumin (MSA).

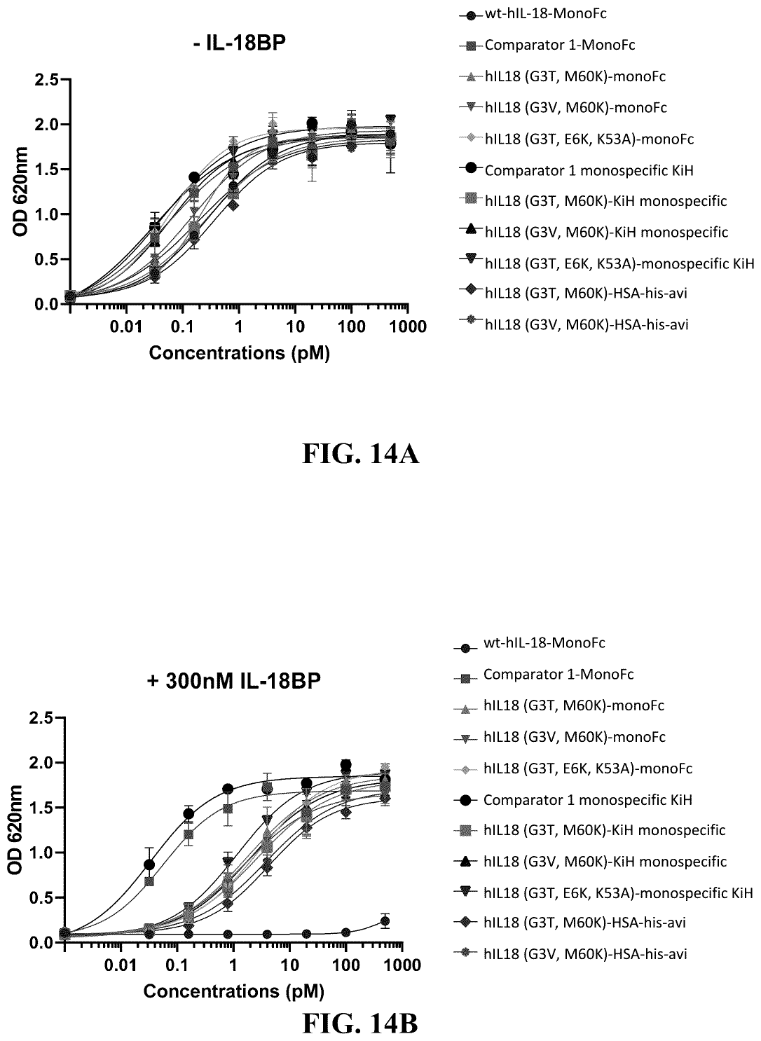

The above recited polypeptides were linked to WT IL-18, the IL-18 Comparator 1, and several IL-18 variants described herein. As shown in FIG. 14A-FIG. 14B and Table 21, all variants retained activity in the HEK-blue assay and were resistant to IL-18BP repression, regardless of which polypeptide was linked to the variant.

TABLE 21
EC50 values for IL-18 conjugate activity from a HEK-blue assay.

| | EC50 (pM) − | EC50 (pM) + |
|---|---|---|
| Description | IL-18BP | 300 nM IL-18BP |
| WT-hIL-18-monoFc | 0.29 | >500 pM |
| Comparator 1-monoFc | 0.05 | 0.06 |
| G3T/M60K-monoFc | 0.21 | 1.78 |
| G3V/M60K-monoFc | 0.15 | 1.57 |
| G3T/E6K/K53A-monoFc | 0.06 | 2.81 |
| Comparator 1-monospecific KiH | 0.03 | 0.03 |
| G3T/M60K-monospecific KiH | 0.27 | 2.64 |
| G3V/M60K-monospecific KiH | 0.06 | 2.01 |
| G3T/E6K/K53A-monospecific KiH | 0.04 | 1.16 |
| G3T/M60K-HSA-his-avi | 0.36 | 4.25 |
| G3V/M60K-HSA-his-avi | 0.19 | 3.83 |
| Comparator 1-HSA-his-avi | 0.28 | 0.27 |

In the table above, “his” corresponds to a 6xHis purification tag and “avi” corresponds to the avi purification tag.

### Additional IL-18 Variants:

Additional IL-18 variants were generated and tested based on the results above with G3 variants, E6 variants, K53 variants, and M60 variants. The new variants were first tested for binding to IL-18Rα and IL-18BP by octet, as described above. Table 22 summarizes the results.

TABLE 22
Binding affinity to IL-18Rα and IL-18BP.

| | IL-18 Rα | hIL18BP (tested up to 10 μM) |
|---|---|---|
| Variant | Kd (nM) | Kd (nM) |
| Comparator 1 | 16 | >10,000 |
| WT | 56 | 0.14 |
| G3V/K53S/M60K | 5310 | >1,000* |
| G3V/K53A/M60K | 488 | >10,000 |
| G3T/K53S/M60K | 5050 | >1,000* |
| G3T/K53A/M60K | 2260 | >1,000* |
| G3V/E6K/K53S/M60K | 324 | >10,000 |
| G3V/E6K/K53A/M60K | 82 | >10,000 |
| G3T/E6K/K53S/M60K | 536 | >10,000 |
| G3T/E6K/K53A/M60K | 222 | >10,000 |

*max concentration tested is 1 uM, did not test up to 10 uM

The binding data indicates that each IL-18 variant retains at least some binding affinity for IL-18Rα while greatly reducing IL-18BP binding affinity.

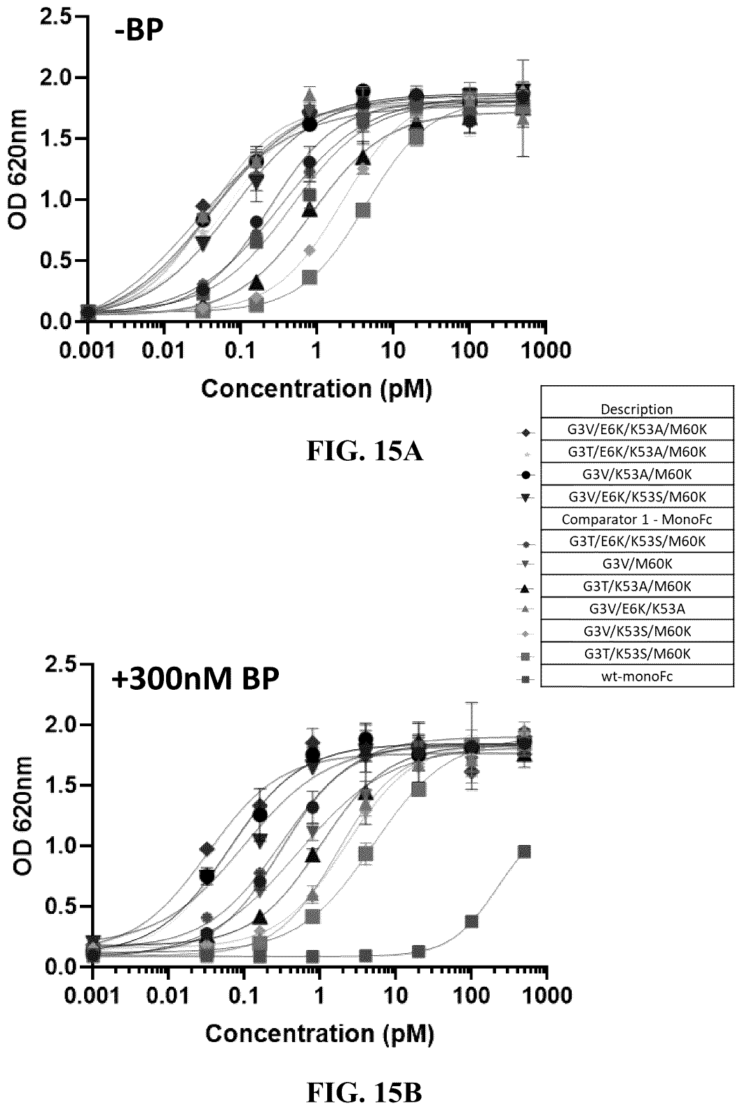

The variants were next tested in the HEK-blue assay. As shown in FIG. 15A, FIG. 15B, Table 23, and Table 24, each of the variants retained robust activity in the presence of IL-18BP, at either 300 nM IL-18BP or 1000 nM IL-18BP.

TABLE 23
EC50 values for IL-18 variant activity from a HEK-blue assay.

| Description | EC50 (pM) − BP | EC50 (pM) + 300 nM BP |
|---|---|---|
| G3V/E6K/K53A/M60K | 0.026 | 0.033 |
| G3T/E6K/K53A/M60K | 0.061 | 0.062 |
| G3V/K53A/M60K | 0.041 | 0.063 |
| G3V/E6K/K53S/M60K | 0.079 | 0.098 |
| Comparator 1 - MonoFc | 0.237 | 0.307 |
| G3T/E6K/K53S/M60K | 0.330 | 0.308 |
| G3V/M60K | 0.042 | 0.507 |
| G3T/K53A/M60K | 0.843 | 1.006 |
| G3V/E6K/K53A | 0.038 | 1.683 |
| G3V/K53S/M60K | 2.107 | 2.184 |
| G3T/K53S/M60K | 4.265 | 4.990 |
| wt-monoFc | 0.458 | 220.800 |

TABLE 24
EC50 values for IL-18 variant activity from a HEK-blue assay.

| Description | EC50 (pM) − BP | EC50 (pM) + 1000 nM BP |
|---|---|---|
| G3V/E6K/K53A/M60K | 0.031 | 0.028 |
| G3T/E6K/K53A/M60K | 0.054 | 0.065 |
| G3V/K53A/M60K | 0.086 | 0.075 |
| G3V/E6K/K53S/M60K | 0.089 | 0.095 |
| Comparator 1 - MonoFc | 0.198 | 0.200 |
| G3T/E6K/K53S/M60K | 0.252 | 0.231 |
| G3V/M60K | 1.430 | 1.051 |
| G3V/E6K/K53A | 0.050 | 1.261 |
| G3T/K53A/M60K | 1.879 | 1.312 |
| G3V/K53S/M60K | 0.021 | 2.606 |
| G3T/K53S/M60K | 5.532 | 4.104 |
| wt-monoFc | 0.495 | 99.750 |

### IL-18 Variant Cytotoxicity:

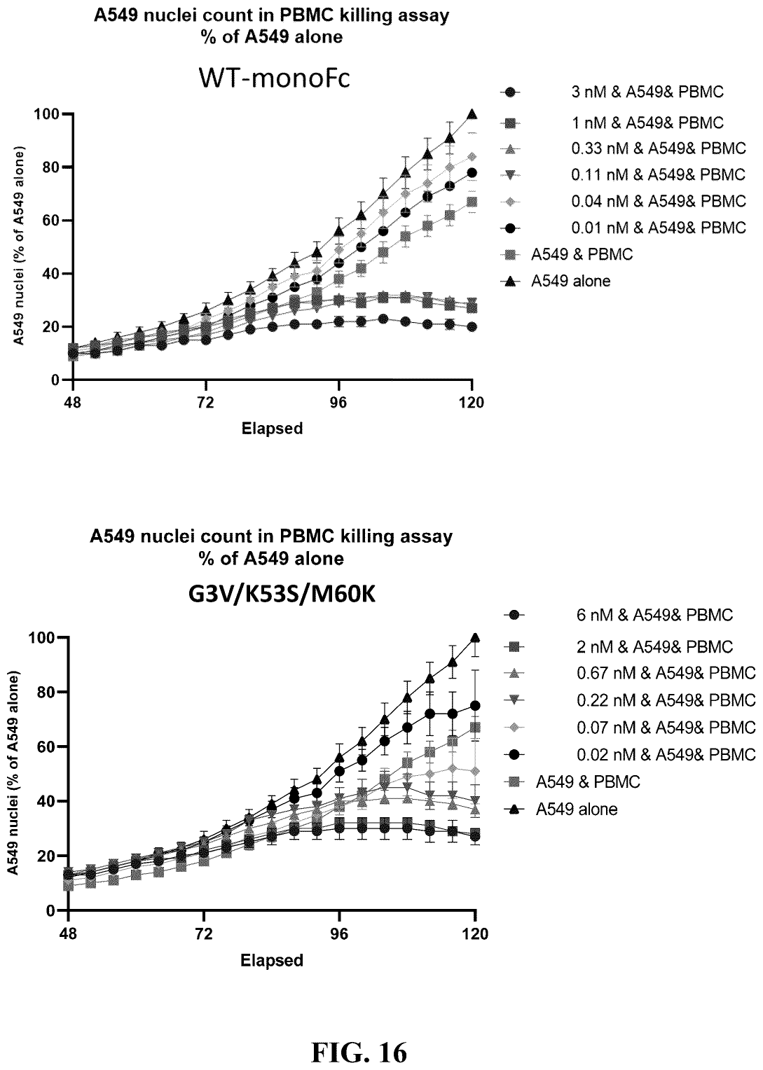

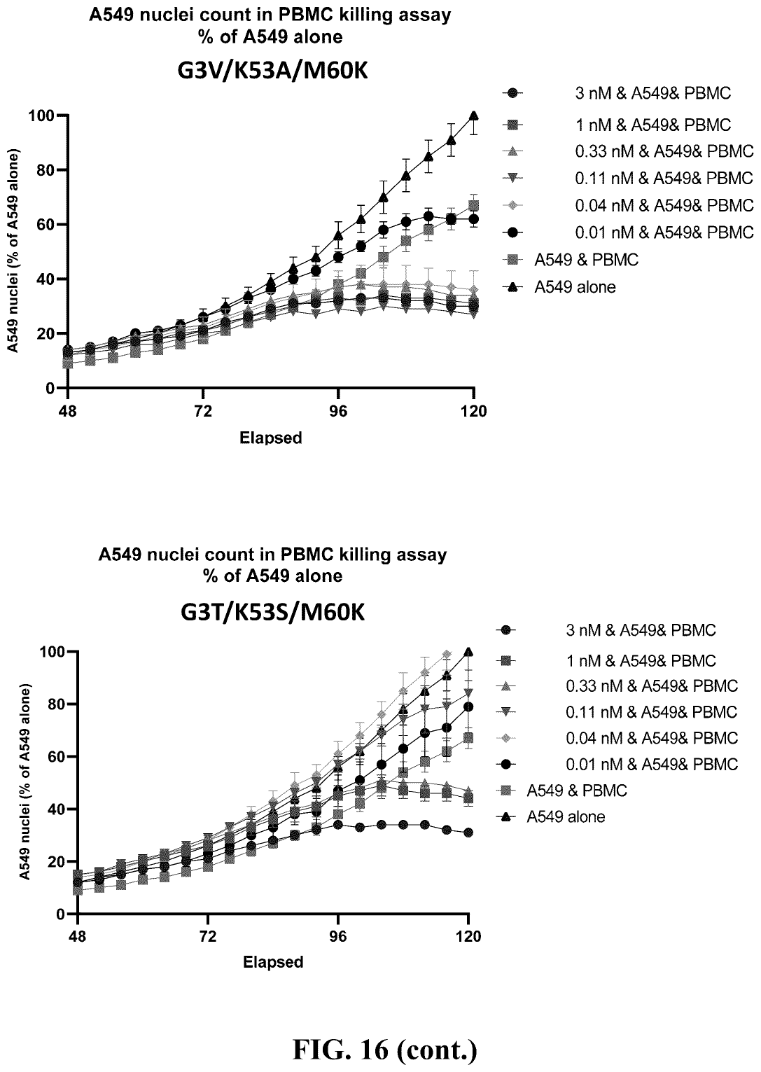

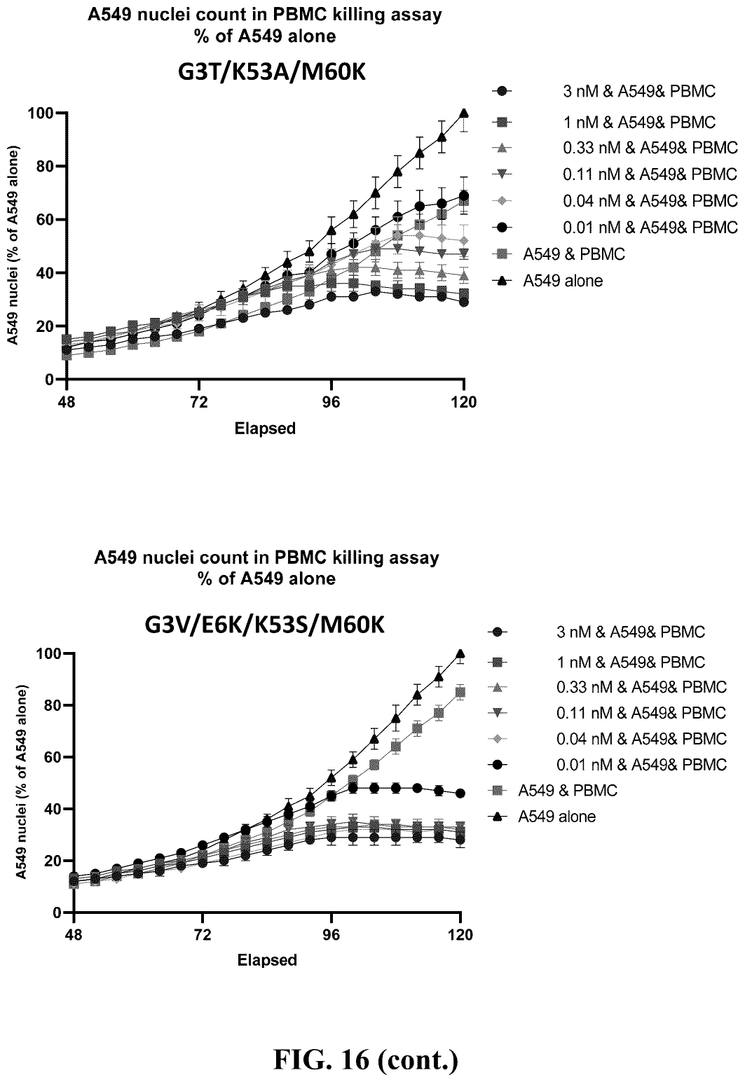

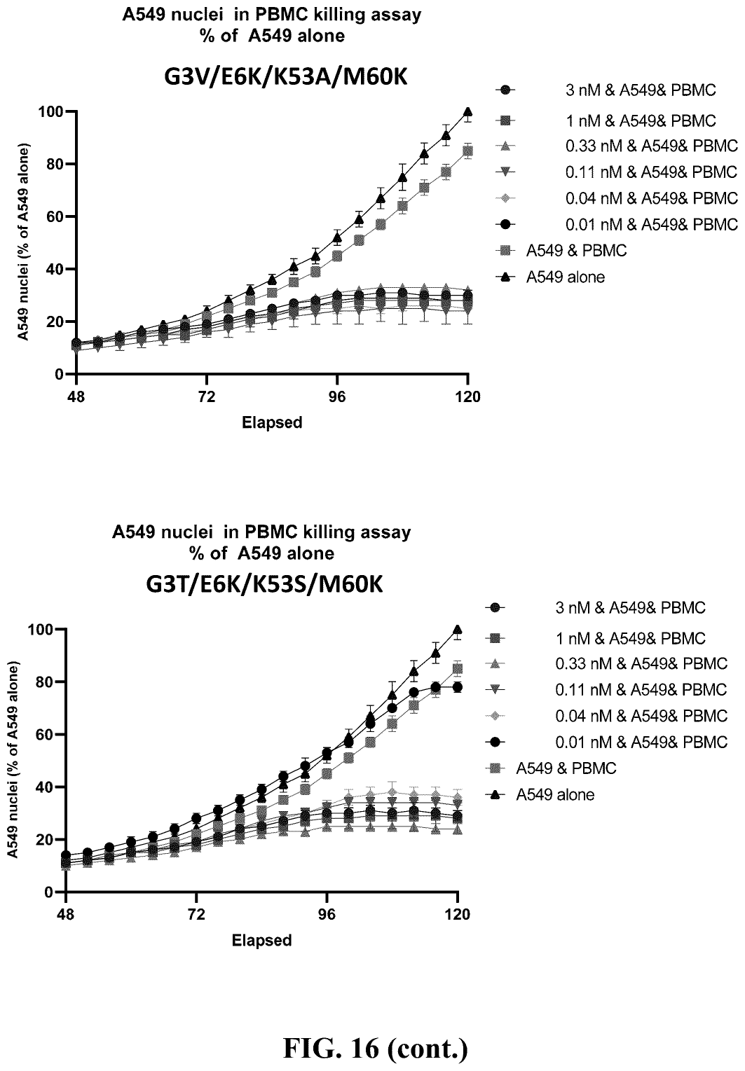

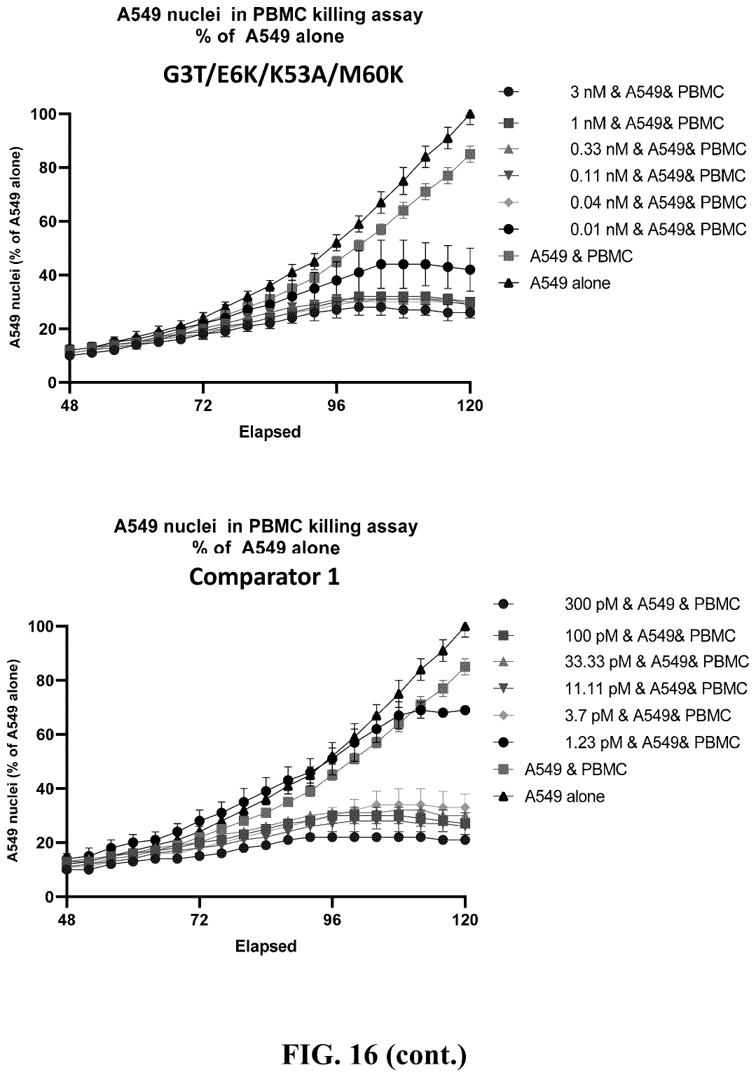

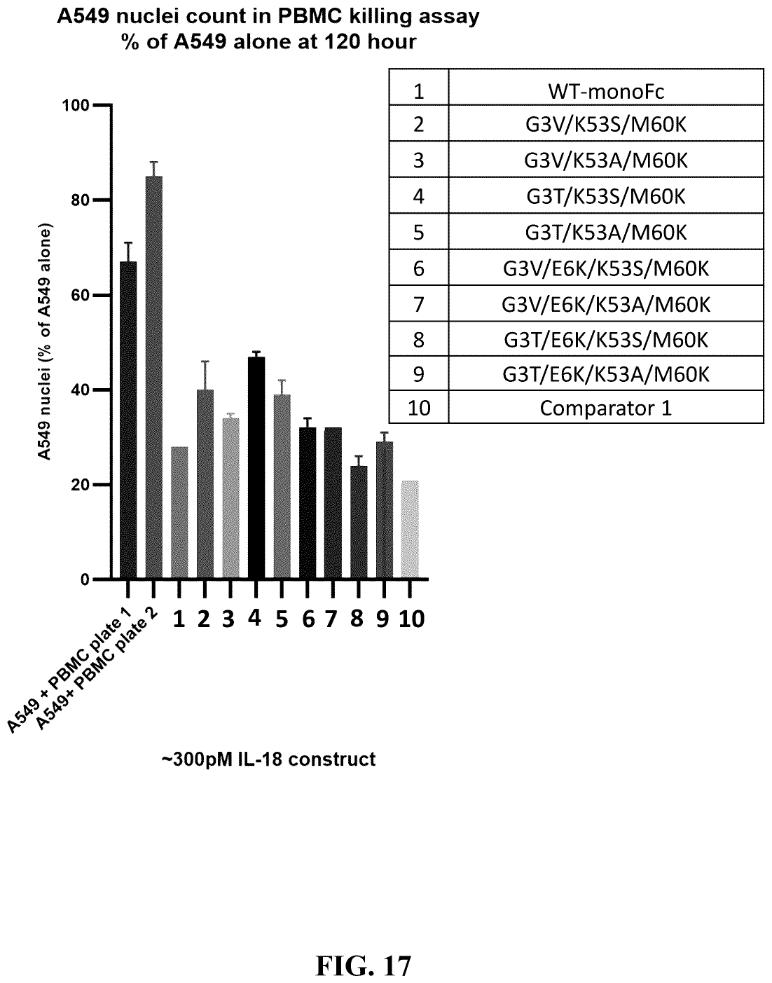

Select IL-18 variants were tested in a A549 cell/PBMC killing assay as described in Example 1. The assay provides a measure of IL-18 activity to stimulate immune cells in the PBMC population to kill the co-cultured A549 cells. As shown in FIG. 16 and FIG. 17, the IL-18 variants displayed the same cytotoxic activity as wild-type IL-18.

### Yeast Display IL-18 Variants

A yeast display strategy for identifying IL-18 variants was employed in parallel with the rational design approach. The yeast display allows for a high diversity of variants across three sites of IL-18 (Site I, II, and III) simultaneously or independently. Eight to twelve amino acids across all 3 sites were selected to balance between diversity and residue selection. Three libraries were then generated to capture the highest diversity. The amino acid positions used in each library (Library A, B, and C) are shown below in Table 25.

TABLE 25
Yeast display library design.

| Library | Site | Residues |
|---|---|---|
| A | II | K53, S55, R104 |
| | I, III | T34, S36, D37, D40, N41, P107, H109 |
| B | II | G3, E6, K53, D54, S55, T63, R104, N91 |
| C | I, III | T34, D35, S36, D37, D40, N41, P107, G108, H109 |

For library A screening, 79 potential IL-18 variants were generated and screened in the HEK blue activity assay, +/−100 nM IL-18BP. Of those 71 variants, 5 variants were full activators of IL-18 signaling in the presence of IL-18BP (ratio −BP/+BP is <1.5 for all 3 data points, DF-1000, DF-100,000, DF-1,000,000), 12 variants were weak full activators of IL-18 signaling in the presence of IL-18BP (also ratio −BP/+BP is <1.5 for all 3 data points, DF-1000, DF-100,000, DF-1,000,000), and 1 variant was a partial activator of IL-18 signaling in the presence of IL-18BP (ratio −BP/+BP is <1.5 for at least 1 data point, DF-1000, DF-100,000, DF-1,000,000). Results of the Library A screen are provided below in Table 26.

TABLE 26
Activity of Library A IL-18 variants.

| | No BP | | | +100 nM BP | | | Ratio (−BP/+BP) | | |
|---|---|---|---|---|---|---|---|---|---|
| | DF- | DF- | DF- | DF- | DF- | DF- | DF- | DF- | DF- |
| Variant | 10000 | 100,000 | 1,000,000 | 10000 | 100,000 | 1,000,000 | 10000 | 100,000 | 1,000,000 |
| hDR-18-monoFc | 2.19 | 2.26 | 2.16 | 2.21 | 2.19 | 2.10 | 0.99 | 1.03 | 1.03 |
| K53S, S55K | 2.00 | 1.35 | 0.50 | 2.03 | 1.41 | 0.53 | 0.98 | 0.96 | 0.95 |
| K53T, S55A | 1.80 | 0.96 | 0.29 | 1.85 | 0.95 | 0.32 | 0.97 | 1.00 | 0.89 |
| K53S, S55G | 1.71 | 0.91 | 0.30 | 1.83 | 0.97 | 0.27 | 0.93 | 0.95 | 1.10 |
| K53T, S55P | 1.42 | 0.59 | 0.16 | 1.53 | 0.65 | 0.21 | 0.93 | 0.91 | 0.79 |
| K53S, S55Y | 1.41 | 0.54 | 0.19 | 1.52 | 0.55 | 0.21 | 0.93 | 0.97 | 0.90 |
| K53T, S55M, R58K | 0.78 | 0.20 | 0.12 | 0.72 | 0.21 | 0.17 | 1.08 | 0.92 | 0.72 |
| S36E, D37E, D40K, | 0.81 | 0.22 | 0.17 | 0.81 | 0.21 | 0.14 | 1.00 | 1.01 | 1.22 |
| N41T, K53T, S55Q, | | | | | | | | | |
| R104H | | | | | | | | | |
| L5W, I22M, K53N, | 0.75 | 0.21 | 0.13 | 0.72 | 0.23 | 0.21 | 1.05 | 0.91 | 0.61 |
| S55Y, Q56H | | | | | | | | | |
| S36Y, D37E, D40X, | 0.71 | 0.18 | 0.11 | 0.87 | 0.21 | 0.16 | 0.81 | 0.87 | 0.66 |
| N41M, K53S, S55L | | | | | | | | | |
| K53T, S55F | 0.67 | 0.20 | 0.16 | 0.60 | 0.18 | 0.25 | 1.12 | 1.09 | 0.64 |
| K53G, S55I | 0.59 | 0.14 | 0.09 | 0.76 | 0.19 | 0.13 | 0.77 | 0.73 | 0.70 |
| K53G, S55V | 0.56 | 0.15 | 0.10 | 0.57 | 0.15 | 0.10 | 0.98 | 1.01 | 1.03 |
| E6D, S36F, K53T, | 0.48 | 0.14 | 0.09 | 0.50 | 0.14 | 0.09 | 0.96 | 1.02 | 0.96 |
| S55M, R58K | | | | | | | | | |
| K53D, S55N | 0.45 | 0.13 | 0.09 | 0.47 | 0.12 | 0.09 | 0.95 | 1.05 | 1.00 |
| T34I, S36T, D40A, | 0.38 | 0.18 | 0.14 | 0.37 | 0.12 | 0.12 | 1.03 | 1.45 | 1.18 |
| N41M, K53T, S55Y | | | | | | | | | |
| K53N, S55I | 0.32 | 0.11 | 0.09 | 0.29 | 0.11 | 0.09 | 1.10 | 1.00 | 0.99 |
| K53W, S55A | 0.28 | 0.13 | 0.11 | 0.31 | 0.16 | 0.13 | 0.89 | 0.84 | 0.84 |
| K53H, S55V | 1.60 | 0.94 | 0.33 | 1.05 | 0.34 | 0.14 | 1.53 | 2.79 | 2.31 |
| K53N, S55M, R104S, | 0.80 | 0.24 | 0.12 | 0.15 | 0.12 | 0.11 | 5.37 | 1.93 | 1.08 |
| P107A | | | | | | | | | |
| L5W, S7Y, K53H, | 0.50 | 0.16 | 0.16 | 0.12 | 0.12 | 0.13 | 4.04 | 1.41 | 1.24 |
| S55M | | | | | | | | | |
| K53Q, D54N, S55M, | 0.46 | 0.13 | 0.09 | 0.21 | 0.11 | 0.09 | 2.21 | 1.16 | 1.06 |
| Q56H | | | | | | | | | |
| K53S, S55F, R104K, | 0.43 | 0.11 | 0.09 | 0.14 | 0.09 | 0.09 | 3.08 | 1.18 | 0.95 |
| H109Y | | | | | | | | | |
| K53Y, S55Y | 0.38 | 0.17 | 0.12 | 0.24 | 0.14 | 0.21 | 1.57 | 1.21 | 0.57 |
| K53Y, S55G | 0.36 | 0.14 | 0.12 | 0.21 | 0.16 | 0.13 | 1.71 | 0.88 | 0.94 |
| T34I, S36V, D40I, | 0.30 | 0.11 | 0.14 | 0.21 | 0.10 | 0.09 | 1.46 | 1.11 | 1.52 |
| N41M, K53G, S55Y | | | | | | | | | |
| WT-monoFc | 2.24 | 2.08 | 1.75 | 0.92 | 0.29 | 0.24 | 2.45 | 7.16 | 7.32 |

TABLE 26
Activity of Library A IL-18 variants.

| | No BP | | | +100 nM BP | | | Ratio (−BP/+BP) | | |
|---|---|---|---|---|---|---|---|---|---|
| | DF- | DF- | DF- | DF- | DF- | DF- | DF- | DF- | DF- |
| Variant | 10000 | 100,000 | 1,000,000 | 10000 | 100,000 | 1,000,000 | 10000 | 100,000 | 1,000,000 |
| T34M, S36T, D37E, D40T, N41M, | 0.29 | 0.21 | 0.19 | 0.14 | 0.12 | 0.12 | 2.13 | 1.79 | 1.63 |
| K53T, S55W | | | | | | | | | |
| D23G, K53Y, S55Y | 0.25 | 0.10 | 0.11 | 0.11 | 0.10 | 0.10 | 2.29 | 1.09 | 1.13 |
| K53Y, S55G, Q56H | 0.26 | 0.15 | 0.11 | 0.30 | 0.21 | 0.18 | 0.86 | 0.70 | 0.63 |
| S36E, D40A, N41M, K53T, S55Y, | 0.24 | 0.11 | 0.09 | 0.25 | 0.11 | 0.09 | 0.97 | 1.02 | 1.01 |
| P107S | | | | | | | | | |
| K53D, S55Q, H109Y | 0.23 | 0.20 | 0.13 | 0.12 | 0.15 | 0.10 | 1.94 | 1.36 | 1.27 |
| T34L, S36V, D40A, N41S, K53G, | 0.22 | 0.19 | 0.14 | 0.12 | 0.12 | 0.14 | 1.81 | 1.60 | 0.97 |
| S55W | | | | | | | | | |
| S7Y, K53H, S55M | 0.21 | 0.14 | 0.18 | 0.16 | 0.15 | 0.18 | 1.33 | 0.94 | 0.98 |
| K53G, S55P, R104K, H109Y | 0.21 | 0.12 | 0.10 | 0.12 | 0.09 | 0.09 | 1.72 | 1.30 | 1.20 |
| K53N, S55A, R104N, P107A, H109Y | 0.21 | 0.13 | 0.13 | 0.13 | 0.12 | 0.14 | 1.64 | 1.10 | 0.93 |
| K53V, S55Q | 0.20 | 0.14 | 0.11 | 0.20 | 0.17 | 0.13 | 1.00 | 0.81 | 0.88 |
| S36E, D37E, D40R, N41G, K53Y, | 0.20 | 0.12 | 0.14 | 0.16 | 0.19 | 0.14 | 1.19 | 0.64 | 1.06 |
| S55F | | | | | | | | | |
| K53D, S55Q, P107M | 0.19 | 0.13 | 0.13 | 0.22 | 0.14 | 0.13 | 0.85 | 0.97 | 1.02 |
| K53Y, S55Y, Q56H | 0.16 | 0.15 | 0.14 | 0.22 | 0.20 | 0.19 | 0.73 | 0.76 | 0.73 |
| K53Y, S55V | 0.16 | 0.20 | 0.16 | 0.16 | 0.14 | 0.13 | 1.03 | 1.44 | 1.27 |
| K53N, S55Y | 0.16 | 0.09 | 0.08 | 0.14 | 0.09 | 0.09 | 1.11 | 1.02 | 0.96 |
| K53S, S55Y, P107M | 0.15 | 0.11 | 0.12 | 0.19 | 0.14 | 0.18 | 0.82 | 0.76 | 0.69 |
| L9V, K53H, S55M | 0.15 | 0.10 | 0.11 | 0.16 | 0.10 | 0.14 | 0.94 | 1.01 | 0.78 |
| K53H, S55H | 0.15 | 0.10 | 0.08 | 0.15 | 0.10 | 0.09 | 1.01 | 0.99 | 0.96 |
| K53H, S55I | 0.14 | 0.10 | 0.09 | 0.16 | 0.11 | 0.10 | 0.92 | 0.90 | 0.89 |
| K53H, S55M, Q56H | 0.14 | 0.09 | 0.08 | 0.15 | 0.10 | 0.09 | 0.97 | 0.96 | 0.92 |
| K53H, S55Q | 0.14 | 0.10 | 0.09 | 0.14 | 0.10 | 0.10 | 1.03 | 1.03 | 0.91 |
| T34E, N41D, K53N, S55R | 0.14 | 0.10 | 0.09 | 0.10 | 0.13 | 0.09 | 1.42 | 0.74 | 1.05 |
| K53L, S55E | 0.14 | 0.10 | 0.09 | 0.12 | 0.09 | 0.09 | 1.12 | 1.07 | 1.01 |
| K53S, S55F, T63S, R104K, H109Y | 0.14 | 0.09 | 0.09 | 0.13 | 0.09 | 0.09 | 1.03 | 1.00 | 0.99 |
| S36W, D37E, D40F, N41I, K53D, | 0.13 | 0.14 | 0.13 | 0.20 | 0.13 | 0.12 | 0.68 | 1.07 | 1.05 |
| S55N | | | | | | | | | |
| K53D, S55T, H109I | 0.13 | 0.09 | 0.08 | 0.12 | 0.09 | 0.09 | 1.10 | 0.97 | 0.98 |
| K53H, S55G | 0.13 | 0.10 | 0.08 | 0.12 | 0.09 | 0.08 | 1.08 | 1.04 | 1.02 |
| K53T, S55G, P107A, H109Q | 0.13 | 0.10 | 0.11 | 0.12 | 0.16 | 0.12 | 1.10 | 0.63 | 0.87 |
| R13S, D23H, R27T, F30L, T34D, | 0.13 | 0.09 | 0.11 | 0.25 | 0.20 | 0.13 | 0.52 | 0.48 | 0.86 |
| S36Y, D37E, M38X, D40R, N41M, | | | | | | | | | |
| K53S, S55L | | | | | | | | | |
| K53D, S55E | 0.13 | 0.09 | 0.09 | 0.11 | 0.09 | 0.08 | 1.22 | 1.07 | 1.08 |
| K53H, S55M | 0.13 | 0.10 | 0.09 | 0.12 | 0.09 | 0.09 | 1.12 | 1.03 | 1.09 |
| K53H, S55M, T63S | 0.13 | 0.10 | 0.09 | 0.18 | 0.12 | 0.11 | 0.72 | 0.83 | 0.83 |
| K53D, S55N, H109I | 0.13 | 0.12 | 0.09 | 0.10 | 0.09 | 0.09 | 1.28 | 1.42 | 0.99 |
| K53G, S55V, R104Y, H109N | 0.13 | 0.12 | 0.10 | 0.11 | 0.14 | 0.10 | 1.17 | 0.90 | 1.02 |
| K53N, S55H, H109F | 0.13 | 0.10 | 0.10 | 0.11 | 0.09 | 0.09 | 1.17 | 1.06 | 1.17 |
| K53H, S55Y, Q56H | 0.12 | 0.10 | 0.09 | 0.12 | 0.09 | 0.09 | 1.05 | 1.03 | 0.96 |
| K53X, S55A | 0.12 | 0.10 | 0.08 | 0.11 | 0.10 | 0.10 | 1.05 | 1.03 | 0.86 |
| K53L, S55G | 0.12 | 0.10 | 0.09 | 0.14 | 0.11 | 0.11 | 0.87 | 0.92 | 0.89 |
| K53H, S55R | 0.12 | 0.10 | 0.09 | 0.12 | 0.09 | 0.09 | 0.97 | 1.09 | 1.00 |
| T34N, S36Y, D40H, N41T, K53G, | 0.12 | 0.10 | 0.09 | 0.11 | 0.09 | 0.09 | 1.08 | 1.02 | 0.97 |
| S55Y | | | | | | | | | |
| T34N, S36Y, D37E, D40Q, N41Q, | 0.11 | 0.09 | 0.09 | 0.10 | 0.10 | 0.09 | 1.11 | 0.96 | 1.00 |
| K53D, S55D | | | | | | | | | |
| T34S, S36L, D37E, D40Y, N41M, | 0.11 | 0.10 | 0.09 | 0.10 | 0.10 | 0.09 | 1.11 | 0.98 | 0.98 |
| K53T, S55K, P107K, H109Q | | | | | | | | | |
| K53H, S55H, N87D, H109I | 0.11 | 0.10 | 0.09 | 0.10 | 0.10 | 0.10 | 1.10 | 0.96 | 0.88 |
| K53D, S55L | 0.11 | 0.09 | 0.09 | 0.10 | 0.09 | 0.09 | 1.02 | 1.03 | 1.01 |
| L15I, K53H, S55M | 0.11 | 0.10 | 0.09 | 0.12 | 0.10 | 0.08 | 0.90 | 0.99 | 1.02 |
| K53G, S55I, R104K, P107Y | 0.10 | 0.09 | 0.09 | 0.11 | 0.09 | 0.09 | 0.99 | 1.00 | 0.95 |
| K8R, L9G, S10C, K53G, S55A, | 0.10 | 0.13 | 0.10 | 0.12 | 0.14 | 0.10 | 0.88 | 0.92 | 1.03 |
| R104G | | | | | | | | | |
| S36Q, D40T, K53P, S55L | 0.09 | 0.13 | 0.08 | 0.09 | 0.09 | 0.09 | 1.09 | 1.44 | 0.90 |
| K53H, S55Y | 0.09 | 0.09 | 0.09 | 0.10 | 0.10 | 0.11 | 0.91 | 0.99 | 0.80 |
| T34E, S36P, D40A, N41G, K53T, | 0.09 | 0.10 | 0.09 | 0.10 | 0.09 | 0.09 | 0.98 | 1.01 | 0.96 |
| S55R | | | | | | | | | |
| S36E, D37E, D40V, N41S, K53Y, | 0.09 | 0.09 | 0.09 | 0.09 | 0.10 | 0.10 | 1.03 | 0.92 | 0.89 |
| S55V, H109Y | | | | | | | | | |
| T34S, S36Y, D37N, D40E, N41Q, | 0.09 | 0.09 | 0.09 | 0.09 | 0.09 | 0.12 | 0.98 | 0.96 | 0.75 |
| K53Y, S55I | | | | | | | | | |
| S36F, N41E, K53W | 0.09 | 0.10 | 0.09 | 0.09 | 0.10 | 0.09 | 1.00 | 1.01 | 0.91 |
| T34N, S36N, D40E, N41G, K53S, | 0.09 | 0.09 | 0.08 | 0.09 | 0.09 | 0.09 | 0.95 | 1.01 | 0.95 |
| S55Q, R104N, H109Y | | | | | | | | | |

The top library A hits were re-screened in a HEK-blue assay, +/−300 nM IL-18BP. Although the hits were not as active as WT IL-18 without IL-18BP, all library A hits were more active with IL-18BP. The results are reported in Table 27.

TABLE 27
Library A activity in HEK-blue re-screening.

| | EC50 | EC50 (pM) + | |
|---|---|---|---|
| | (pM) − | 300 nM | |
| Description | BP | BP | Ratio |
| Comparator 1-monoFc | 0.17 | 0.23 | 1.4 |
| K53S/S55K | 21.79 | 18.05 | 0.8 |
| K53S/S55G | 33.41 | 102.6 | 3.1 |
| K53T/S55P | 103.3 | 133.4 | 1.3 |
| K53S/S55Y | 69.97 | 136.4 | 1.9 |
| K53T/S55A | 67.43 | 139.8 | 2.1 |
| K53H/S55V | 45.5 | >200 | 10.1 |
| WT-monoFc | 0.52 | >500 | 946.7 |

For library B screening, 65 potential IL-18 variants were generated and screened in the HEK blue activity assay, +/−100 nM IL-18BP. Of those 65 variants, 12 variants were full activators of IL-18 signaling in the presence of IL-18BP, 10 variants were weak full activators of IL-18 signaling in the presence of IL-18BP, and 17 variants were partial activators of IL-18 signaling in the presence of IL-18BP. Results of the Library B screen are provided below in Table 28.

TABLE 28
Activity of Library B IL-18 variants.

| | No BP | | | +100 nM BP | | | Ratio (−BP/+BP) | | |
|---|---|---|---|---|---|---|---|---|---|
| | DF- | DF- | DF- | DF- | DF- | DF- | DF- | DF- | DF- |
| Variant | 100000 | 500000 | 2500000 | 1000002 | 5000003 | 25000004 | 1000005 | 5000006 | 25000007 |
| hDR-18-monoFc | 2 | 1.91 | 1.49 | 2.01 | 1.95 | 1.59 | 0.99 | 0.98 | 0.93 |
| G3L, E6Y, D54T, T63S, N91Y, | 2.14 | 2.02 | 1.69 | 2.04 | 1.92 | 1.45 | 1.05 | 1.05 | 1.17 |
| R104Q | | | | | | | | | |
| G3L, E6S, D54T, T63S, N91Y, | 2.11 | 1.98 | 1.73 | 2.06 | 1.95 | 1.44 | 1.02 | 1.02 | 1.2 |
| R104Q | | | | | | | | | |
| G3P, E6K, D54Y, S55P, Q56H, | 1.63 | 1.1 | 0.53 | 1.69 | 1.08 | 0.54 | 0.96 | 1.02 | 0.98 |
| T63S, N91S, R104Q | | | | | | | | | |
| G3D, E6G, K53G, D54A, T63S, | 1.36 | 0.72 | 0.21 | 1.33 | 0.73 | 0.19 | 1.02 | 0.98 | 1.14 |
| N91Y, R104Q | | | | | | | | | |
| G3Q, E6H, K53T, D54S, S55Y, | 1.52 | 0.95 | 0.31 | 1.48 | 0.88 | 0.29 | 1.03 | 1.08 | 1.08 |
| N91Y, R104K | | | | | | | | | |
| G3P, E6H, N91L | 1.45 | 0.9 | 0.29 | 1.52 | 0.93 | 0.33 | 0.95 | 0.97 | 0.87 |
| G3P, E6K, D54Y, S55P, T63S, | 1.75 | 1.32 | 0.72 | 1.8 | 1.35 | 0.77 | 0.97 | 0.97 | 0.93 |
| N91S, R104Q | | | | | | | | | |
| G3P, E6K, D54Y, S55P, T63S, | 1.49 | 0.94 | 0.36 | 1.51 | 0.81 | 0.39 | 0.99 | 1.16 | 0.93 |
| N91S, R104Q, Q154R | | | | | | | | | |
| G3Q, E6Q, K53T, D54W, S55F, | 1.23 | 0.54 | 0.17 | 1.14 | 0.45 | 0.14 | 1.08 | 1.21 | 1.2 |
| N91Y | | | | | | | | | |
| G3P, E6H, D54A, S55P, Q56K, | 1.2 | 0.63 | 0.17 | 1.24 | 0.61 | 0.19 | 0.97 | 1.03 | 0.9 |
| T63S, N91M | | | | | | | | | |
| G3P, E6K, D54R, S55P, T63A, | 1.31 | 0.67 | 0.22 | 1.34 | 0.73 | 0.28 | 0.98 | 0.92 | 0.79 |
| N91V | | | | | | | | | |
| G3P, E6V, K53A, D54W, S55P, | 1.18 | 0.52 | 0.19 | 1.21 | 0.57 | 0.18 | 0.98 | 0.9 | 1.04 |
| T63S, N91L, R104K | | | | | | | | | |
| R104K | | | | | | | | | |
| G3P, E6G, D54T, S55W, N91E, | 0.35 | 0.13 | 0.07 | 0.33 | 0.12 | 0.08 | 1.09 | 1.06 | 0.97 |
| R104K | | | | | | | | | |
| G3R, L5W, E6H, D54Y, Q56H, | 0.39 | 0.12 | 0.08 | 0.32 | 0.11 | 0.08 | 1.25 | 1.09 | 1.01 |
| N91R | | | | | | | | | |
| G3S, E6Q, K53S, D54Y, S55H, | 0.49 | 0.16 | 0.08 | 0.51 | 0.2 | 0.1 | 0.95 | 0.81 | 0.88 |
| N91L, R104Q | | | | | | | | | |
| G3T, E6Q, K53G, D54G, S55H, | 0.58 | 0.18 | 0.12 | 0.49 | 0.18 | 0.11 | 1.19 | 1.03 | 1.14 |
| T63A, S65T, N91R, R104Q | | | | | | | | | |
| G3P, E6K, K53M, D54W, S55N, | 0.64 | 0.21 | 0.08 | 0.65 | 0.2 | 0.09 | 0.98 | 1.07 | 0.91 |
| T63S, N91I | | | | | | | | | |
| G3P, E6R, D54W, S55Q, T63S, | 0.74 | 0.25 | 0.09 | 0.89 | 0.26 | 0.12 | 0.83 | 0.96 | 0.78 |
| N91T, R104H | | | | | | | | | |
| G3P, E6V, K53A, D54W, S55P, | 0.56 | 0.18 | 0.08 | 0.6 | 0.19 | 0.09 | 0.94 | 0.96 | 0.91 |
| T63S, N91L, R104K, Q154R | | | | | | | | | |
| G3Y, E6S, K53S, D54W, S55L, | 0.7 | 0.24 | 0.15 | 0.78 | 0.26 | 0.12 | 0.9 | 0.95 | 1.22 |
| N91I | | | | | | | | | |
| G3N, E6S, K53Q, D54W, S55R, | 0.46 | 0.14 | 0.14 | 0.46 | 0.15 | 0.14 | 0.99 | 0.93 | 1.02 |
| N91T, R104T | | | | | | | | | |
| G3Y, E6S, K53S, D54W, S55L, | 0.68 | 0.23 | 0.1 | 0.62 | 0.23 | 0.12 | 1.09 | 0.99 | 0.84 |
| T63S, N91I | | | | | | | | | |
| G3Q, E6N | 1.95 | 1.93 | 1.85 | 1.88 | 1.52 | 0.84 | 1.04 | 1.27 | 2.2 |
| G3Q, E6S, S55V, T63A, N91T | 1.94 | 1.84 | 1.61 | 1.15 | 0.46 | 0.15 | 1.68 | 4.03 | 10.7 |
| G3Q, E6H, D54N, S55I, T63S, | 1.89 | 1.9 | 1.64 | 1.8 | 1.43 | 0.71 | 1.05 | 1.33 | 2.31 |
| N91V | | | | | | | | | |
| G3Q, E6K, D54S, S55P, T63S, | 1.92 | 1.92 | 1.4 | 1.55 | 1.15 | 0.45 | 1.24 | 1.67 | 3.14 |
| N91S, R104Q | | | | | | | | | |
| G3I, E6D, D54A, S55F, N91Y, | 2.1 | 1.96 | 1.34 | 2.05 | 1.45 | 0.74 | 1.02 | 1.35 | 1.81 |
| R104H | | | | | | | | | |
| G3Q, E6N, D54Y, S55P, T63S, | 1.37 | 0.75 | 0.28 | 0.9 | 0.42 | 0.14 | 1.52 | 1.76 | 1.97 |
| N91S, R104H | | | | | | | | | |
| G3Y, E6N, N91Y, R104S | 1.87 | 1.79 | 1.54 | 1.78 | 1.2 | 0.6 | 1.05 | 1.5 | 2.59 |
| G3Q, E6G, D54N, S55H, T63V, | 1.99 | 1.82 | 1.01 | 1.83 | 1.38 | 0.7 | 1.09 | 1.32 | 1.44 |
| N91M, R104T | | | | | | | | | |
| G3E, E6G, N91M, R104A | 2.05 | 2 | 1.54 | 1.93 | 1.41 | 0.53 | 1.06 | 1.41 | 2.92 |
| G3S, E6G, D54V, S55K, T63S, | 1.95 | 1.77 | 1.26 | 1.64 | 0.85 | 0.32 | 1.19 | 2.08 | 3.88 |
| N91Y, R104T | | | | | | | | | |
| G3L, E6Q, T63S, N91L, R104T | 2.12 | 2.01 | 1.85 | 1.81 | 1.15 | 0.46 | 1.17 | 1.75 | 4 |
| G3E, E6H, R27S, D54A, S55T, | 2.08 | 1.83 | 1.14 | 1.8 | 1.11 | 0.41 | 1.16 | 1.65 | 2.79 |
| T63S, N91F, R104S | | | | | | | | | |
| E6G, D54A, S55K, T63S, N91K, | 2.2 | 2.05 | 1.61 | 1.89 | 1.33 | 0.49 | 1.17 | 1.54 | 3.3 |
| Q114R | | | | | | | | | |
| G3I, E6Q, N91M, R104Y | 2.12 | 2 | 1.53 | 1.84 | 0.96 | 0.29 | 1.15 | 2.07 | 5.26 |
| G3L, E6Q, D54Y, S55P, T63S, | 1.66 | 0.9 | 0.37 | 0.75 | 0.19 | 0.1 | 2.23 | 4.67 | 3.8 |
| N91S, R104Q | | | | | | | | | |
| G3D, E6Q, R27S, K53R, D54A, | 1.02 | 0.41 | 0.13 | 0.52 | 0.15 | 0.09 | 1.95 | 2.81 | 1.5 |
| S55R, T63S, N91Q | | | | | | | | | |
| E6D, D54T, S55W, T63S, N91S, | 1.27 | 0.55 | 0.18 | 0.37 | 0.15 | 0.1 | 3.41 | 3.62 | 1.93 |
| R104T | | | | | | | | | |
| wt-monoFc | 1.94 | 1.43 | 0.77 | 0.11 | 0.08 | 0.07 | 17.4 | 17.4 | 10.5 |
| N91S, R104H | 1.34 | 0.61 | 0.19 | 0.09 | 0.08 | 0.11 | 14.7 | 7.35 | 1.72 |
| G3V, E6Q, T34P, D54M, S55F, | 0.97 | 0.41 | 0.13 | 0.14 | 0.08 | 0.08 | 6.88 | 4.95 | 1.77 |
| N91R, R104H | | | | | | | | | |
| G3S, E6Q, K53N, D54T, S55N, | 0.24 | 0.11 | 0.08 | 0.23 | 0.11 | 0.11 | 1.05 | 1.03 | 0.71 |
| T63A, I80T, N91S | | | | | | | | | |
| G3H, E6Q, K53N, D54S, S55P, | 0.18 | 0.09 | 0.07 | 0.21 | 0.12 | 0.08 | 0.86 | 0.78 | 0.91 |
| T63A, N91I, R104S | | | | | | | | | |
| G3L, E6N, I22M, T63V, N91T, | 0.08 | 0.07 | 0.07 | 0.09 | 0.09 | 0.07 | 0.8 | 0.82 | 0.97 |
| R104P | | | | | | | | | |
| G3Q, E6D, K53S, D54I, S55E, | 0.09 | 0.07 | 0.07 | 0.12 | 0.09 | 0.11 | 0.73 | 0.8 | 0.68 |
| T63S, N91S, R104Q | | | | | | | | | |
| K53T, D54H, S55V, T63A, N91K | 0.28 | 0.12 | 0.12 | 0.23 | 0.1 | 0.07 | 1.18 | 1.24 | 1.58 |
| G3P, E6K, K53T, D54Y, S55V, | 0.26 | 0.11 | 0.07 | 0.32 | 0.11 | 0.08 | 0.81 | 1.01 | 0.93 |
| T63I, N91I | | | | | | | | | |
| G3P, E6G, D54P, S55W, N91E, | 0.09 | 0.08 | 0.07 | 0.09 | 0.1 | 0.07 | 0.98 | 0.84 | 0.92 |
| R104K | | | | | | | | | |
| E6N, D54N, S55N, N91H, R104P | 0.08 | 0.07 | 0.07 | 0.08 | 0.08 | 0.09 | 0.99 | 0.93 | 0.78 |
| G3P, E6K, K53N, D54S, S55P, | 0.07 | 0.08 | 0.07 | 0.07 | 0.07 | 0.07 | 0.92 | 1.03 | 0.95 |
| T63S, N91S, R104Q | | | | | | | | | |
| G3R, E6D, D54A, S55Q, T63S, | 0.07 | 0.08 | 0.07 | 0.07 | 0.08 | 0.07 | 0.98 | 0.98 | 0.96 |
| N91K, R104P | | | | | | | | | |
| G3P, E6N, D54Y, T63S, N91S, | 0.21 | 0.1 | 0.07 | 0.25 | 0.1 | 0.08 | 0.86 | 1.02 | 0.89 |
| R104Q | | | | | | | | | |
| G3P, E6S, K53R, D54Y, S55P, | 0.15 | 0.08 | 0.07 | 0.14 | 0.08 | 0.07 | 1.07 | 0.97 | 0.99 |
| T63I, N91K | | | | | | | | | |
| G3P, E6R, K53T, D54Y, S55I, | 0.14 | 0.08 | 0.07 | 0.15 | 0.08 | 0.07 | 0.93 | 0.96 | 0.99 |
| T63V, N91G, R104K | | | | | | | | | |
| G3Q, E6N, K53T, D54A, N91K, | 0.07 | 0.08 | 0.07 | 0.07 | 0.09 | 0.08 | 1.03 | 0.89 | 0.95 |
| R104P | | | | | | | | | |
| G3P, E6D, K53T, D54N, T63A, | 0.07 | 0.07 | 0.07 | 0.07 | 0.08 | 0.07 | 0.95 | 0.92 | 1.01 |
| N91T, R104P | | | | | | | | | |
| E6T, D54P, S55K, R104S | 0.17 | 0.09 | 0.1 | 0.18 | 0.09 | 0.08 | 0.98 | 0.99 | 1.3 |
| G3S, E6H, K53Y, D54P, S55Y, | 0.09 | 0.09 | 0.07 | 0.07 | 0.08 | 0.08 | 1.18 | 1.1 | 0.95 |
| T63S, N91A, R104S | | | | | | | | | |
| G3L, E6Q, T63S, N91L, R104P | 0.15 | 0.09 | 0.1 | 0.1 | 0.08 | 0.07 | 1.44 | 1.11 | 1.34 |
| G3P, E6S, K53N, D54Y, S55P, | 0.07 | 0.07 | 0.07 | 0.07 | 0.07 | 0.07 | 1.03 | 0.94 | 0.95 |
| T63I, N91K, R104H | | | | | | | | | |
| G3Q, E6H, S7Y, D23A, R27S, | 0.08 | 0.08 | 0.07 | 0.07 | 0.08 | 0.07 | 1.12 | 1.01 | 1.04 |
| D54S, S55R, T63F, N91F, R104Q | | | | | | | | | |
| G3P, E6I, K53N, D54W, S55P, | 0.07 | 0.08 | 0.07 | 0.08 | 0.09 | 0.07 | 0.91 | 0.91 | 0.97 |
| T63S, N91L, R104Q | | | | | | | | | |
| G3L, E6Q, T63H, N91M, R104H | 0.16 | 0.09 | 0.11 | 0.08 | 0.07 | 0.07 | 2.02 | 1.16 | 1.47 |
| G3N, E6Y, K53Q, D54W, S55R, | 0.07 | 0.07 | 0.07 | 0.07 | 0.08 | 0.07 | 1.02 | 0.93 | 1.03 |
| N91T, R104P | | | | | | | | | |
| G3E, E6A, K53Q, D54W, S55V, | 0.07 | 0.08 | 0.08 | 0.08 | 0.08 | 0.08 | 0.97 | 1.03 | 0.99 |
| T63M, N91M, R104N | | | | | | | | | |

The 12 full activator IL-18 variants from library B hits were re-screened in a HEK-blue assay, +/−300 nM IL-18BP. The results are reported in Table 29. All 12 full activator IL-18 variants displayed similar or better activity compared to WT IL-18 while also retaining activity in the presence of IL-18BP, consistent with the initial screen.

TABLE 29
Library B full activator activity in HEK-blue re-screening.

| | EC50 | EC50 (pM) + | |
|---|---|---|---|
| | (pM) − | 300 nM | |
| Description | BP | BP | Ratio |
| Comparator 1-monoFc | 0.07 | 0.07 | 1.0 |
| WT-monoFc | 3.49 | 143 | 41.0 |
| G3D, E6G, K53G, D54A, T63S, N91Y, | 2.62 | 3.11 | 1.2 |
| R104Q | | | |
| G3L, E6S, D54T, T63S, N91Y, R104Q | 0.02 | 0.51 | 25.5 |
| G3L, E6Y, D54T, T63S, N91Y, R104Q | 0.03 | 0.37 | 12.3 |
| G3P, E6H, D54A, S55P, Q56K, T63S, | 5.4 | 8.23 | 1.5 |
| N91M | | | |
| G3P, E6H, N91L | 3.21 | 5.34 | 1.7 |
| G3Q, E6Q, K53T, D54W, S55F, N91Y | 2.36 | 2.54 | 1.1 |
| Comparator 1-monoFc | 0.28 | 0.27 | 1.0 |
| WT-monoFc | 0.7 | >500 | 346.0 |
| G3P, E6K, D54R, S55P, T63A, N91V | 9.07 | 10.12 | 1.1 |
| G3P, E6K, D54Y, S55P, Q56H, T63S, | 1.58 | 2.99 | 1.9 |
| N91S, R104Q | | | |
| G3P, E6K, D54Y, S55P, T63S, N91S, | 0.73 | 1.18 | 1.6 |
| R104Q | | | |
| G3P, E6K, D54Y, S55P, T63S, N91S, | 1.09 | 2.58 | 2.4 |
| R104Q, Q154R | | | |
| G3P, E6V, K53A, D54W, S55P, T63S, | 8.21 | 15.81 | 1.9 |
| N91L, R104K | | | |
| G3Q, E6H, K53T, D54S, S55Y, N91Y, | 2.63 | 4.69 | 1.8 |
| R104K | | | |

The 17 partial activator IL-18 variants from library B hits were re-screened in a HEK-blue assay, +/−100 nM IL-18BP. The results are reported in Table 30. All 17 partial activator IL-18 variants displayed similar or better activity compared to WT IL-18 while also retaining activity in the presence of IL-18BP, consistent with the initial screen.

TABLE 30
Library B partial activator activity in HEK-blue re-screening.

| | EC50 | EC50 (pM) + | |
|---|---|---|---|
| | (pM) − | 100 nM | |
| Description | BP | BP | Ratio |
| Comparator 1-monoFc | 0.25 | 0.22 | 0.9 |
| WT-monoFc | 0.51 | >500 | 520.4 |
| G3Q, E6S, S55V, T63A, N91T | 0.2 | 35.9 | 179.5 |
| G3Q, E6K, D54S, S55P, T63S, | 0.27 | 7.45 | 27.6 |
| N91S, R104Q | | | |
| G3Y, E6N, N91Y, R104S | 0.09 | 2.89 | 32.1 |
| G3E, E6G, N91M, R104A | 0.17 | 3.49 | 20.5 |
| G3S, E6G, D54V, S55K, T63S, | 0.21 | 4.87 | 23.2 |
| N91Y, R104T | | | |
| G3L, E6Q, T63S, N91L, R104T | 0.03 | 1.67 | 55.7 |
| Comparator 1-monoFc | 0.33 | 0.23 | 0.7 |
| WT-monoFc | 0.43 | 155 | 360.5 |
| G3E, E6H, R27S, D54A, S55T, | 0.34 | 3.01 | 8.9 |
| T63S, N91F, R104S | | | |
| E6G, D54A, S55K, T63S, N91K, | 0.1 | 2.17 | 21.7 |
| Q114R | | | |
| G3I, E6Q, N91M, R104Y | 0.07 | 3.77 | 53.9 |
| G3L, E6Q, D54Y, S55P, T63S, | 1.35 | 54.68 | 40.5 |
| N91S, R104Q | | | |
| G3D, E6Q, R27S, K53R, D54A, | 4.22 | 25.68 | 6.1 |
| S55R, T63S, N91Q | | | |
| E6D, D54T, S55W, T63S, N91S, | 3.42 | 62.77 | 18.4 |
| R104T | | | |
| Comparator 1-monoFc | 0.38 | 0.31 | 0.8 |
| WT-monoFc | 0.6 | >500 | 337.5 |
| G3Q, E6N, D54Y, S55P, T63S, N91S, | 9.63 | 34.72 | 3.6 |
| R104H | | | |
| G3Q, E6N | 0.05 | 8.61 | 172.2 |
| G3Q, E6H, D54N, S55I, T63S, N91V | 0.31 | 5.23 | 16.9 |
| G3I, E6D, D54A, S55F, N91Y, R104H | 0.45 | 3.41 | 7.6 |
| G3Q, E6G, D54N, S55H, T63V, | 0.99 | 2.66 | 2.7 |
| N91M, R104T | | | |

Two yeast display IL-18 variants were next tested in a PBMC IFN gamma expression assay. The two variants had better activity as measured by IFN gamma expression than WT IL-18 and retained that activity in the presence of 300 nM IL-18BP. Table 31 below reports the results.

TABLE 31
Yeast display IL-18 variant activity in a PBMC IFN gamma assay.

| | EC50 |
|---|---|
| IL-18 type | (pM) |
| WT-monoFc | 96.78 |
| WT-monoFc + 300 nM BP | 611 |
| hIL18(G3P, E6K, D54Y, S55P, T63S, N91S, R104Q)-monoFc | 19.52 |
| hIL18(G3P, E6K, D54Y, S55P, T63S, N91S, | 74.54 |
| R104Q)-monoFc + 300 nM BP | |
| hIL18(G3P, E6K, D54Y, S55P, T63S, N91S, R104Q, | 85.63 |
| Q154R)-monoFc | |
| hIL18(G3P, E6K, D54Y, S55P, T63S, N91S, R104Q, | 64.09 |
| Q154R)-monoFc + 300 nM BP | |
| Comparator 1 | 4.863 |
| Comparator 1 + 300 nM BP | 8.776 |

Human PBMC Assay with Purification Tag-Free IL-18 Variants

Various IL-18 variants were test in the human PBMC stimulated IFN gamma assay as described in Example 1. The following variants were employed, each variant being linked to an antibody Fc domain with the knob-in-hole mutations (KiH).

TABLE 32
IL-18 variant descriptions in a PBMC IFN gamma assay.

| Variant | |
|---|---|
| Number | Description |
| 1 | hIL18 (G3V, E6K, K53A, M60K)- KiH |
| 2 | hIL18 (G3T, E6K, K53A, M60K) - KiH |
| 3 | hIL18 (G3V, E6K, K53S, M60K) - KiH |
| 4 | hIL18 (G3T, K53A, M60K) - KiH |
| 5 | hIL18 (G3V, K53A, M60K) - KiH |
| 6 | hIL18 (G3T, E6K, K53S, M60K) - KiH |
| 7 | hIL18 (G3T, K53S, M60K) - KiH |
| 8 | hIL18 (G3V, K53S, M60K) - KiH |

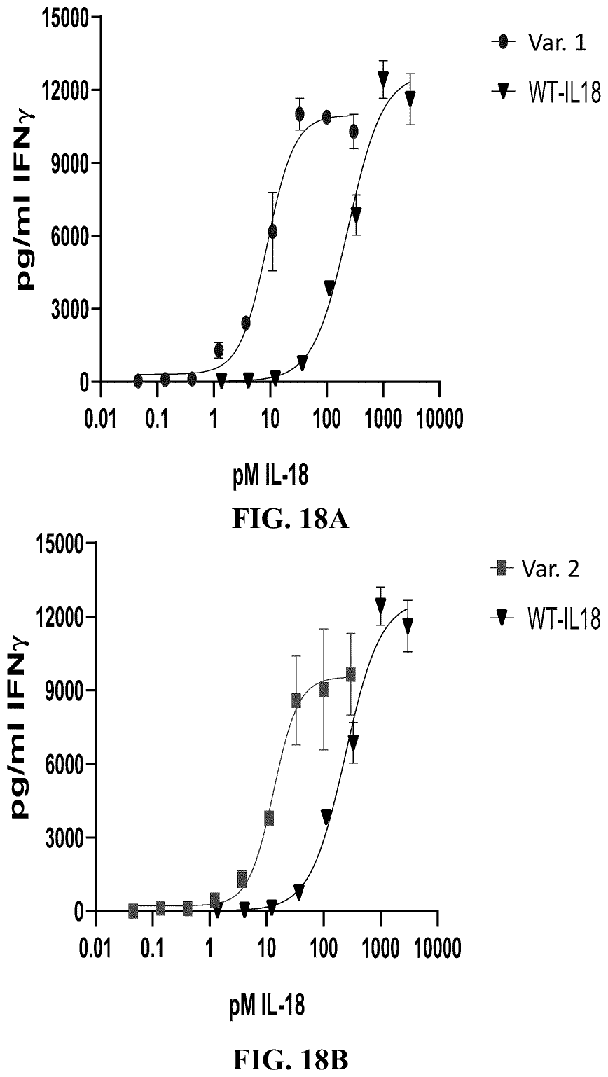

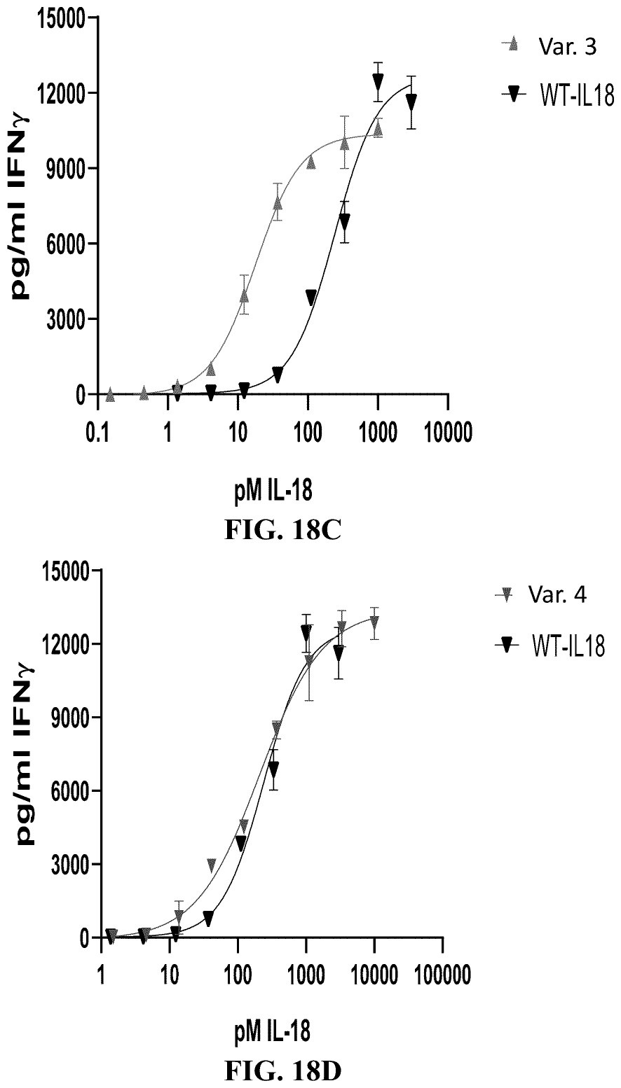

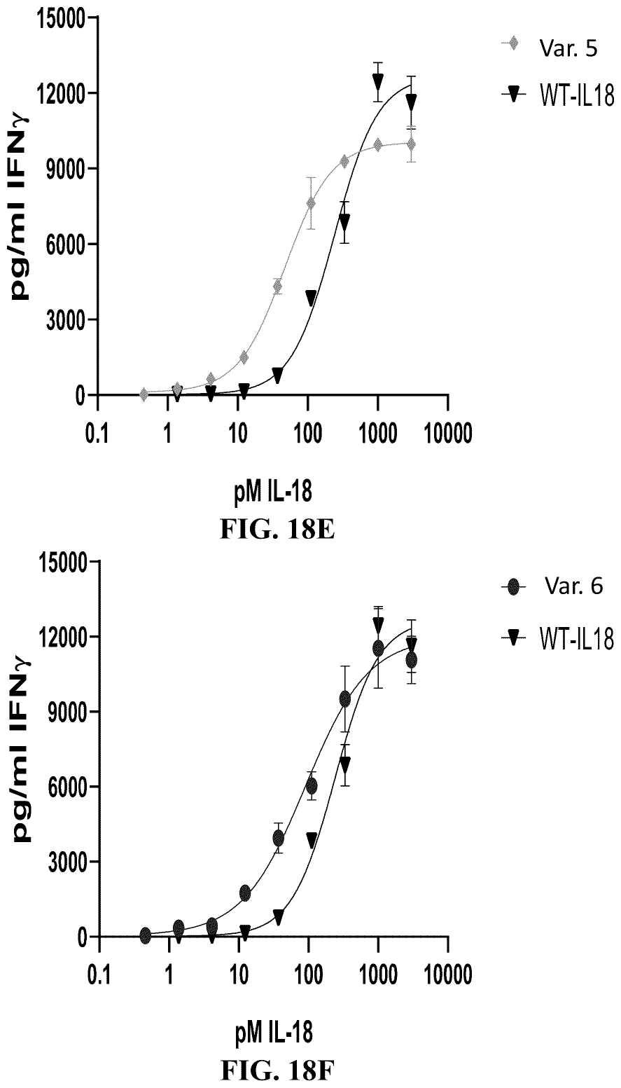

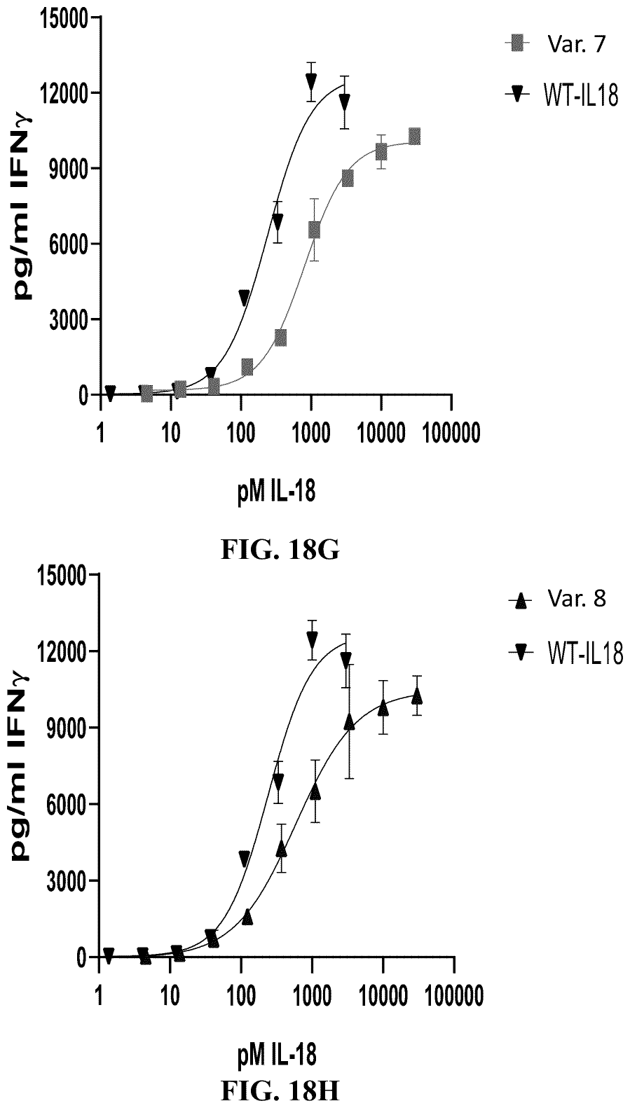

As shown in FIG. 18A-H, each IL-18 variant potently stimulated IFN gamma production. The results of FIG. 18A-H graphically depict run 1. A second and third run were performed. The results of the assay are shown in Table 33 and Table 34.

TABLE 33
EC50 (pM) values of IL-18 variants
in the PBMC IFN gamma assay.

| Variant ID | 1 | 2 | 3 | average | SEM |
|---|---|---|---|---|---|
| 1 | 8.831 | 3.206 | 4.332 | 5.46 | 0.99 |
| 2 | 13.32 | 14.04 | 20.72 | 16.03 | 1.36 |
| 3 | 17.83 | 21.62 | 10.87 | 16.77 | 1.82 |
| 4 | 208.9 | 214.1 | 162.5 | 195.17 | 9.47 |
| 5 | 47.29 | 74.99 | 59.44 | 60.57 | 4.63 |
| 6 | 90.26 | 121.4 | 41.57 | 84.41 | 13.41 |
| 7 | 809.6 | 1124 | 784 | 905.87 | 63.11 |
| 8 | 589.3 | 534.9 | 603.4 | 575.87 | 12.06 |
| WT-IL18 | 240.7 | 105.2 | 161.8 | 169.23 | 22.69 |
| 1 | 0.04 | 0.03 | 0.03 | 0.03 | 0.002 |
| 2 | 0.06 | 0.13 | 0.13 | 0.11 | 0.01 |
| 3 | 0.07 | 0.21 | 0.07 | 0.12 | 0.03 |
| 4 | 0.87 | 2.04 | 1.00 | 1.30 | 0.21 |
| 5 | 0.20 | 0.71 | 0.37 | 0.43 | 0.09 |
| 6 | 0.37 | 1.15 | 0.26 | 0.60 | 0.16 |
| 7 | 3.36 | 10.68 | 4.85 | 6.30 | 1.29 |
| 8 | 2.45 | 5.08 | 3.73 | 3.75 | 0.44 |
| WT-IL18 | 1 | 1 | 1 | 1.00 | 0.00 |

1. An interleukin 18 (IL-18) polypeptide comprising an amino acid substitution at position G3 of SEQ ID NO: 1.

2. The IL-18 polypeptide of claim 1, wherein the amino acid substitution comprises G3T, G3V, G3I, G3R, G3E, G3S, G3A, G3F, G3H, or G3K.

3. The IL-18 polypeptide of claim 1, further comprising one or more amino acid substitutions at positions L5, E6, S7, K8, L9, 510, R13, L15, 122, D23, R27, F30, T34, 536, D37, M38, D40, N41, M51, K53, Q56, 555, R58, G59, M60, T63, N87, N91, T95, Q103, R104, 5105, P107, H109, D110, N111, M113, Q114, Q154, N155, E156, and D157.

4-5. (canceled)

6. The IL-18 polypeptide of claim 3, wherein the amino acid substitution comprises
L5I or L5W;
E6K, E6R, E6Y, E61, E6S, E6Q, E6L, E6M, E6T, E6A, E6D, E6G, E6H, or E6N;
S7Y;
K8R or K8H;
L9V or L9G;
S10C;
R13S;
L15I;
I22M;
D23G, D23H, or D23A;
R27T or R27S;
F30L;
T34E, T34I, T34N, T34S, T34D, T34M, or T34P;
S36F, S36P, S36V, S36N, S36Y, S36L, S36E, S36Q, S36W, or S36T;
D37E or D37N;
M38X, wherein X corresponds to a deletion of M38;
D40A, D40I, D40E, D40H, D40Q, D40Y, D40T, D40F, D40R, D40K, or D40X, wherein X corresponds to a deletion of D40;
N41G, N41D, N41M, N41T, N41Q, N41S, N41E, or N41I;
M51K, M51R, M51Y, or M51F;
K53A, K53S, K53T, K53H, K53N, K53T, K53G, K53S, K53D, K53Y, K53W, K53L, K53Q, K53V, K53R, or K53X, wherein X corresponds to a deletion of K53;
S55M, S55R, S55Y, S55Q, S55D, S55K, S55I, S55V, S55L, S55A, S55E, S55N, S55P, S55G, S55W, or S55F;
Q56E, Q56H, or Q56K;
R58K;
G59D;
M60K, M60Q, or M60N;
T63A, T63S, T63V, T63H, T63M, or T36I;
N87D;
N91R, N91L, N91M, N91E, N91A, N91S, N91V, N91W, N91I, N91Y, N91Q, N91F, N91K, N91H, N91T, N91E, N91G, or N91F;
T95K or T95H;
Q103A or Q103V;
R104N, R014K, R104Y, R014S, R104G, R104Q, R104N, R104A, R104T, R104H, or R104P;
S105R or S105F;
P107K, P107S, P107Y, P107M, P107A, or P107N;
H109Y, H109Q, H109I, H109F, or H109N;
D110N, D110T, D110Q, D110H, or D110R;
N111F, N111Q, or N111D;
M113V, M113D, M113K, M113N, M113Q, M113S, M113A, M113E, or M113I;
Q114R;
Q154R;
N155L, N155K, N155S, or N155Y;
E156L; and
D157E or D157R.

7-25. (canceled)

26. The IL-18 polypeptide of claim 1, comprising an amino acid sequence selected from the group consisting of an amino acid sequence with at least 90% identity to an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

27. The IL-18 polypeptide of claim 1, comprising a binding affinity to interleukin 18 binding protein (IL-18 BP) that is about 1 nM or weaker than the binding affinity of an IL-18 polypeptide of SEQ ID NO: 1 to IL-18 BP.

28. (canceled)

29. The IL-18 polypeptide of claim 1, comprising a binding affinity to IL-18 BP of about 1 nM to about 1000 nM and the IL-18 polypeptide retains binding affinity to interleukin 18 receptor alpha (IL-18Rα).

30. (canceled)

31. The IL-18 polypeptide of claim 29, wherein the binding affinity ratio of IL-18Rα binding affinity to IL-18BP binding affinity is less than the binding affinity ratio of an IL-18 polypeptide of SEQ ID NO: 1, optionally wherein the binding affinity ratio of IL-18Rα binding affinity to IL-18BP binding affinity is about 0.01 to about 50.

32-33. (canceled)

34. An interleukin 18 (IL-18) polypeptide comprising an amino acid substitution at position D157 of SEQ ID NO: 1.

35-38. (canceled)

39. The IL-18 polypeptide of claim 34, wherein the amino acid substitution comprises
G3T, G3V, G3I, G3R, G3E, G3 S, G3A, G3F, G3H, or G3K;
L5I or L5W;
E6K, E6R, E6Y, E61, E6S, E6Q, E6L, E6M, E6T, E6A, E6D, E6G, E6H, or E6N;
S7Y;
K8R or K8H;
L9V or L9G;
S10C;
R13S;
L15I;
I22M;
D23G, D23H, or D23A;
R27T or R27S;
F30L;
T34E, T34I, T34N, T34S, T34D, T34M, or T34P;
S36F, S36P, S36V, S36N, S36Y, S36L, S36E, S36Q, S36W, or S36T;
D37E or D37N;
M38X, wherein X corresponds to a deletion of M38;
D40A, D40I, D40E, D40H, D40Q, D40Y, D40T, D40F, D40R, D40K, or D40X, wherein X corresponds to a deletion of D40;
N41G, N41D, N41M, N41T, N41Q, N41S, N41E, or N41I;
M51K, M51R, M51Y, or M51F;
K53A, K53S, K53T, K53H, K53N, K53T, K53G, K53S, K53D, K53Y, K53W, K53L, K53Q, K53V, K53R, or K53X, wherein X corresponds to a deletion of K53;
S55M, S55R, S55Y, S55Q, S55D, S55K, S55I, S55V, S55L, S55A, S55E, S55N, S55P, S55G, S55W, or S55F;
Q56E, Q56H, or Q56K;
R58K;
G59D;
M60K, M60Q, or M60N;
T63A, T63S, T63V, T63H, T63M, or T36I;
N87D;
N91R, N91L, N91M, N91E, N91A, N91S, N91V, N91W, N91I, N91Y, N91Q, N91F, N91K, N91H, N91T, N91E, N91G, or N91F;
T95K or T95H;
Q103A or Q103V;
R104N, R014K, R104Y, R014S, R104G, R104Q, R104N, R104A, R104T, R104H, or R104P;
S105R or S105F;
P107K, P107S, P107Y, P107M, P107A, or P107N;
H109Y, H109Q, H109I, H109F, or H109N;
D110N, D110T, D110Q, D110H, or D110R;
N111F, N111Q, or N111D;
M113V, M113D, M113K, M113N, M113Q, M113S, M113A, M113E, or M113I;
Q114R;
Q154R;
N155L, N155K, N155S, or N155Y; and
E156L.

40-57. (canceled)

58. The IL-18 polypeptide of claim 1, wherein the IL-18 polypeptide is linked to an antibody Fc domain or serum albumin through an amino acid linker, optionally:
wherein the amino acid linker comprises GGSGGGGSGGGSGGGGSGGGGSGGGSGG, GGGGSGGGGSGGGGS, GGGSGGGGSG GGSGGGGSGG, GGSGG, or GGS; and
wherein the antibody Fc domain comprises one or more heterodimerization mutations.

59-65. (canceled)

66. The IL-18 polypeptide of claim 62, wherein the antibody Fc domain comprises a first Fc polypeptide chain and a second Fc polypeptide chain.

67-72. (canceled)

73. The IL-18 polypeptide of claim 66, wherein the IL-18 polypeptide is linked to any one or more of SEQ ID NO: 315, SEQ ID NO: 316, SEQ ID NO: 317, and SEQ ID NO: 318 via an amino acid linker.

74. (canceled)

75. The IL-18 polypeptide of claim 1, wherein the IL-18 polypeptide comprises an N-terminal leader sequence.

76. (canceled)

77. A pharmaceutical composition comprising the IL-18 polypeptide of claim 1, and a pharmaceutically acceptable carrier or diluent.

78. A polynucleotide sequence that encodes the IL-18 polypeptide according to claim 1.

79. An expression vector comprising the polynucleotide sequence of claim 78.

80. A host cell comprising the expression vector of claim 79.

81. A method of producing the IL-18 polypeptide of claim 1, comprising culturing the host cell of claim 80 under conditions to express the IL-18 polypeptide.

82. (canceled)

83. A method of treating cancer in a patient in need thereof comprising administering to the patient a therapeutically effective amount of the pharmaceutical composition of claim 77 or the IL-18 polypeptide of claim 1.
